(19) United States
(12) Patent Application Publication
(10) Pub. No.: US 2026/0207252 A1
(43) Pub. Date: Jul. 23, 2026

(54) HEART VALVE ANNULOPLASTY

(71) Applicant: Bio ReFine Ltd., Or Yehuda (IL)

(72) Inventors: Dvir KEREN, Tel Aviv (IL); Boaz SHENHAV, Tel-Aviv (IL)

(73) Assignee: Bio ReFine Ltd., Or Yehuda (IL)

(21) Appl. No.: 19/141,078

(22) PCT Filed: Dec. 19, 2023
(86) PCT No.: PCT/IL2023/051282

§ 371 Date: Jun. 19, 2025

Related U.S. Application Data
(60) Provisional application No. 63/433,510, filed on Dec. 19, 2022.
(60) Provisional application No. 63/523,680, filed on Jun. 28, 2023.

(51) Int. Cl. A61B 18/14; A61B 18/00; A61B 18/12; A61B 90/00

(52) U.S. Cl. CPC A61B 18/1492; A61B 18/1206; A61B 90/03; A61B 2018/00077; A61B 2018/00178; A61B 2018/00202; A61B 2018/00291; A61B 2018/00369; A61B 2018/00577; A61B 2018/0091; A61B 2018/1405; A61B 2018/1475; A61B 2090/034

(57) ABSTRACT

Annuloplasty devices, configured to mechanically distort valve annulus tissue while providing structurally disruptive energy to induce plastic deformation. In some embodiments, mechanical distortion comprises vacuum-induced attachment to tissue, optionally with or without additional manipulation to pull or stretch tissue. In some embodiments, mechanical distortion comprises torsional deformation of tissue. Optionally, the torsional deformation is limited by the device to a predetermined angle of twist, for example in a range of 60°-120°. In some embodiments, torsional deformation comprises rotating an axis extending between tissue-embedded pins of the device from a first angle oblique to the local circumferential direction to a second such angle, through an intermediate angle which is perpendicular to the local circumferential direction. In some embodiments, a non-penetrating transducer delivers the structurally disruptive energy via a surface contacting the valve annulus tissue.

### RELATED APPLICATION

This application claims the benefit of priority under 35 USC § 119(e) of U.S. Provisional Patent Application No. 63/433,510 filed on Dec. 19, 2022, and of U.S. Provisional Ser. No. 63/523,680 filed on Jun. 28, 2023; the contents of each of which are all incorporated by reference as if fully set forth herein in their entirety.

### FIELD AND BACKGROUND OF THE INVENTION

The present invention, in some embodiments thereof, relates to the field of structural heart disease, and more particularly, but not exclusively, to heart valve annuloplasty.

Patients who suffer from insufficient heart valve function (for example, of the mitral valve) may undergo implantation of an annuloplasty ring, sutured to the heart valve's fibrous annulus tissue. The aim is to shrink and/or stabilize the valve's perimeter. The procedure may be carried out as an open heart surgery, or in with some devices via an intravascular (transcatheter) approach. As the valve's perimeter is shrunk, the valve's leaves get closer, therefore achieving a better sealing (coaptation) to reduce or eliminate valve regurgitation.

International Patent Publication No. WO2002/097130, filed on May 4, 2021, and naming as inventors the inventors of the present disclosure, is hereby incorporated herein by reference in its entirety. International Patent Publication No. WO2002/097130 describes annuloplasty devices using structurally disruptive energy coupled with mechanical manipulation, and methods of using them.

### SUMMARY OF THE INVENTION

According to an aspect of some embodiments of the present disclosure, there is provided an annuloplasty device configured to deliver structurally disruptive energy to a tip terminating a distal end of a catheter body of the device, the annuloplasty device including: a housing of the tip extending between a proximal side connected with the catheter body, and a distal side; and a plurality of pins, each pin: extending distally from a position anchored to the tip within the housing, and sized and sharp to penetrate collagenous tissue of a valve annulus of a human heart; wherein the pins are configured to move by rotation relative to the valve annulus with sufficient force to twist a portion of the collagenous tissue by at least 45°, while the pins are inserted into the collagenous tissue; and wherein movement of the inserted pins to twist the collagenous tissue also rotates the pins and the collagenous tissue relative to the housing.

According to some embodiments of the present disclosure, the movement of the inserted pins rotates collagenous tissue extending along an axis connecting two of the plurality of pins.

According to some embodiments of the present disclosure, the annuloplasty device is configured to limit rotation of the tissue extending along the axis to a predetermined maximum rotation relative to the housing of less than 180°.

According to some embodiments of the present disclosure, the predetermined maximum rotation is less than about 110°.

According to some embodiments of the present disclosure, the predetermined maximum rotation is greater than about 70°.

According to some embodiments of the present disclosure, the plurality of pins consists of exactly two pins.

According to some embodiments of the present disclosure, the two of the plurality of pins are positioned with a distance between their axial centers of 1-2 mm.

According to some embodiments of the present disclosure, the annuloplasty device includes a control actuatable from a proximal side of the catheter body to induce the twisting of the portion of collagenous tissue and the movement of the pins relative to a proximal-distal axis of the housing.

According to some embodiments of the present disclosure, the control includes an element adjusted by manual manipulation, and the annuloplasty device limits rotation of the pins and the collagenous tissue relative to the housing by limiting movement of the manually manipulated element.

According to some embodiments of the present disclosure, the control operates by releasing stored energy to perform the rotation, and the annuloplasty device limits rotation of the pins and the collagenous tissue relative to the housing by limiting an amount of the stored energy. According to some embodiments of the present disclosure, a distal face of the housing includes at least one aperture through which at least one of the pins extends; and the annuloplasty device limits rotation of the pins and the collagenous tissue relative to the housing by interference between at least one of the pins and a circumference of the aperture.

According to some embodiments of the present disclosure, the distal face of the housing includes a plurality of apertures, through each of which a respective pin extends; and the annuloplasty device limits rotation of the pins and collagenous tissue relative to the housing by interference between at least one of the pins and the circumference of its respective aperture.

According to some embodiments of the present disclosure, rotation of the pins relative to the housing is limited by an interfering contact with a stop element within the housing.

According to some embodiments of the present disclosure, the interfering contact with stop element includes interference between an assembly which rotates the pins, and a portion of the housing of the assembly acting as the stop element.

According to some embodiments of the present disclosure, the rotation of the plurality of pins relative to the housing exerts at least 0.01 N·m of torque on the collagenous tissue.

According to some embodiments of the present disclosure, the plurality of pins exert enough torque on the collagenous tissue to drag a portion of it through a rotation about as large as the rotation of an axis extending between two of the pins, the rotation being relative to a proximal-distal axis of the housing.

According to some embodiments of the present disclosure, force to actuate the rotation is transmitted along the catheter body by rotation of a control member.

According to some embodiments of the present disclosure, the annuloplasty device limits the rotation by limiting rotation of the control member.

According to some embodiments of the present disclosure, force to actuate the rotation is transmitted along the catheter body by longitudinal translation of a control member through the catheter body.

According to some embodiments of the present disclosure, the annuloplasty device limits the rotation by limiting the translation of the control member.

According to some embodiments of the present disclosure, the pins remain at a fixed distance from one another during the rotation.

According to some embodiments of the present disclosure, the pins change their distance from each other during the rotation.

According to some embodiments of the present disclosure, the structurally disruptive energy includes RF energy transmitted to the tip along a conductive wire.

According to some embodiments of the present disclosure, the pins are electrodes, interconnected with a power connection on a proximal side of the annuloplasty device through the conductive wire.

According to some embodiments of the present disclosure, the conductive wire is operable to transmit the RF energy to an electrode positioned to contact tissue alongside the pins, when the pins are inserted into the collagenous tissue.

According to some embodiments of the present disclosure, the pins are retractable and extendable relative to the housing.

According to some embodiments of the present disclosure, the annuloplasty device includes a steering sheath, through which the catheter is configured to advance to reach target collagenous tissue.

According to some embodiments of the present disclosure, the steering sheath is configured to move coordinately to adopt a curl and bend out of a plane of the curl as the steering sheath is actuated.

According to some embodiments of the present disclosure, the steering sheath is configured to bend from a straightened configuration to form a segment of the steering sheath into a helical shape.

According to some embodiments of the present disclosure, the helical shape includes a distal segment of the steering sheath, attached to a more proximal segment of the steering sheath, and the more proximal segment of the steering sheath bends from the straightened configuration to form a curvature, while remaining substantially within a plane of the curvature.

According to some embodiments of the present disclosure, the plurality of pins includes a radiopaque material, and the housing includes at least one radiopaque marker, positioned alongside the pins to indicate a state of distal advance of the pins relative to the radiopaque marker.

According to some embodiments of the present disclosure, the at least one radiopaque marker of the housing includes two radiopaque markers positioned on opposite sides of the housing to indicate an orientation of the housing according to the apparent distance of the two radiopaque markers when viewed in a fluoroscopic image.

According to some embodiments of the present disclosure, the annuloplasty device includes an arrangement of electrical contacts in electrical communication with an indicator, configured to indicate a position of the plurality of pins relative to the housing, based on relative positions of the electrical contacts.

According to some embodiments of the present disclosure, the arrangement of electrical contacts includes a first at least one electrical contact fixed to the housing, and a second at least one electrical contact which moves with the pins; wherein illumination of the indicator indicates a state of contact between the first and second at least one electrical contacts.

According to an aspect of some embodiments of the present disclosure, there is provided an annuloplasty device configured to deliver structurally disruptive energy to a tip terminating a distal end of a catheter body, the annuloplasty device including: a first pin and a second pin, each pin: extending distally from a position anchored to the tip, and sized and sharp to penetrate collagenous tissue of a human heart valve annulus; wherein the first pin and second pins are affixed to first and second supports, respectively; and wherein the first support occupies a lumen of the second support, and the first and second supports rotate relative to each other.

According to some embodiments of the present disclosure, the first and second supports move the pins relative to each other with sufficient force to twist a portion of the collagenous tissue by at least 45°, while the pins are inserted into the collagenous tissue.

According to some embodiments of the present disclosure, rotation of the first and second supports relative to each other is limited to a maximum of less than 180°.

According to some embodiments of the present disclosure, the structurally disruptive energy includes RF energy transmitted to the tip along a conductive wire.

According to some embodiments of the present disclosure, the pins are electrodes, interconnected with a power connection on a proximal side of the annuloplasty device through the conductive wire.

According to some embodiments of the present disclosure, the pins are retractable and extendable relative to a sheath, through which the first and second supports are configured to advance to reach target collagenous tissue.

According to some embodiments of the present disclosure, the sheath includes a steering mechanism.

According to an aspect of some embodiments of the present disclosure, there is provided an annuloplasty device configured to deliver structurally disruptive energy to a tip terminating a distal end of a catheter body, the annuloplasty device including: an aperture at the tip: configured to be in pressure communication through the catheter body with a controllable vacuum source, and shaped, sized, and positioned for placement against collagenous tissue of a human heart valve annulus, and, upon activation of vacuum from the vacuum source, for attachment to and deformation of a portion of the collagenous tissue; and a transducer: attached to a conductive wire configured to transmit the structurally disruptive energy to the transducer from an electrical energy source, and positioned within a range of the aperture sufficient to conduct the structurally disruptive energy into the deformed portion of collagenous tissue.

According to some embodiments of the present disclosure, the aperture is elongated.

According to some embodiments of the present disclosure, the transducer is an electrode.

According to some embodiments of the present disclosure, the transducer includes an acoustic energy transducer or a heat energy transducer.

According to some embodiments of the present disclosure, a smallest radius of curvature of the aperture is equal to or larger than about 0.25 mm.

According to some embodiments of the present disclosure, the transducer at least partially defines a circumference of the aperture.

According to some embodiments of the present disclosure, the transducer is positioned where it contacts a surface of the portion of collagenous tissue drawn into the aperture.

According to some embodiments of the present disclosure, the transducer is positioned where it contacts a surface of the portion of collagenous tissue adjacent to at least a circumferential portion of the aperture.

According to some embodiments of the present disclosure, the transducer includes portions positioned on at least two opposing sides of the aperture.

According to some embodiments of the present disclosure, the transducer and the aperture are configured to move relative to each other along a proximal-to-distal axis.

According to an aspect of some embodiments of the present disclosure, there is provided a method of annuloplasty of a human heart valve annulus, the method including: attaching an annuloplasty device tip to a portion of collagenous tissue of the valve annulus; pulling the attached portion of the annuloplasty device tip while also pushing against the tissue with a transducer, the combined pulling and pushing acting to deform the tissue between the transducer and the attached portion; and delivering structurally disruptive energy through the transducer so that the attached portion undergoes plastic deformation toward its deformed shape.

According to some embodiments of the present disclosure, the attaching includes vacuum attachment to the portion of collagenous tissue.

According to some embodiments of the present disclosure, the pulling includes pulling the portion of collagenous tissue into a recess.

According to an aspect of some embodiments of the present disclosure, there is provided a method of annuloplasty of a human heart valve annulus, the method including: placing a distal surface of a tip of an annuloplasty device against a portion of collagenous tissue of the valve annulus, the distal surface including a surface of a transducer configured to deliver structurally disruptive energy; attaching to the collagenous tissue to form an attachment therewith; distorting the collagenous tissue by manipulation of the attachment; and delivering structurally disruptive energy through the surface of the transducer to plastically deform the attached portion.

According to some embodiments of the present disclosure, the distorting pulls a first portion of the collagenous tissue to a first position more proximal than a second position, while also holding a second portion of the collagenous tissue at the second position.

According to some embodiments of the present disclosure, the second position includes two regions, each on an opposite side of the first portion.

According to an aspect of some embodiments of the present disclosure, there is provided a method of annuloplasty of a human heart valve annulus, the method including: positioning a transducer of an annuloplasty device to contact a superficial surface of a portion of collagenous tissue of the valve annulus; and while the transducer remains in contact with the superficial surface: inserting a plurality of pins to penetrate the superficial surface; moving the pins to distort the collagenous tissue; and delivering structurally disruptive energy through the transducer.

According to some embodiments of the present disclosure, positioning the transducer also positions at least one aperture adjacent to the transducer in contact with the superficial surface, and the plurality of pins extend from the at least one aperture when inserting the plurality of pins. According to an aspect of some embodiments of the present disclosure, there is provided a method of annuloplasty of a human heart valve annulus, the method including: inserting first and second pins of an annuloplasty device into a portion of collagenous tissue of the valve annulus so that an axis extending through and between them and along a surface of the collagenous tissue has a first orientation; moving at least one of the first and second pins to rotate the axis extending through and between them to a second orientation; and providing structurally disruptive energy to plastically deform tissue distorted by the moving; wherein the first and second pins insert into the collagenous tissue of the valve annulus at an angle within 55° of an average plane of a whole circumference of the valve annulus.

According to an aspect of some embodiments of the present disclosure, there is provided a method of annuloplasty of a human heart valve annulus, the method including: inserting first and second pins of an annuloplasty device into a portion of collagenous tissue of the valve annulus so that an axis extending through and between them and along a surface of the collagenous tissue has a first orientation; moving at least one of the first and second pins to rotate the axis extending through and between them to a second orientation; and providing structurally disruptive energy to plastically deform tissue distorted by the moving; wherein a plane parallel to a height axis of the valve annulus is within about 15° of bisecting an angle defined by the first and second orientations.

According to some embodiments of the present disclosure, the height axis extends from a first side of the valve annulus to a second side of the valve annulus, and is perpendicular to a plane with maximally uniform distance from the circumference of the valve annulus.

According to some embodiments of the present disclosure, the attaching the annuloplasty device tip to the collagenous tissue of the valve annulus includes attaching to a circumference of the valve annulus at an angle within 55° of an average plane of the circumference.

According to some embodiments of the present disclosure, the attaching the tip of the annuloplasty device to the portion of collagenous tissue of the valve annulus includes attaching to a circumference of the valve annulus at an angle within 55° of an average plane of the circumference.

According to some embodiments of the present disclosure, the positioning of the transducer to contact superficial surface of the portion of collagenous tissue of the valve annulus includes approaching and contacting a circumference of the valve annulus at an angle within 55° of an average plane of the circumference.

According to some embodiments of the present disclosure, the inserting first and second pins of the annuloplasty device also inserts the pins at angle within 55° of an average plane of a circumference of the valve annulus.

According to some embodiments of the present disclosure, the interfering contact with stop element includes interference between an assembly which rotates the pins, and a portion of the housing of the assembly acting as the stop element.

According to some embodiments of the present disclosure, the method includes measuring impedance during the providing, and halting the providing upon sensing an impedance above a threshold, the threshold being selected from within the range of 1800-2500 Ω.

According to some embodiments of the present disclosure, the threshold is 2250 Ω.

According to some embodiments of the present disclosure, the method includes approaching the portion of collagenous tissue while measuring impedance, and performing the inserting while measuring an impedance in a range between 220 Ω and 500 Ω.

According to some embodiments of the present disclosure, the method includes measuring impedance during the inserting, and beginning the moving after the inserting upon measuring an impedance in a range between 240 Ω and 360 Ω.

According to an aspect of some embodiments of the present disclosure, there is provided a steering sheath for an annuloplasty device, configured to interconvert, upon receiving steering control, between a straightened configuration and a curved configuration, wherein the curved configuration includes a helical shape at a distal end of the steering sheath.

According to some embodiments of the present disclosure, the steering sheath includes a segment leading distally to the helical shape which assumes a curvature in the curved configuration that remains substantially within a plane of the curvature.

According to an aspect of some embodiments of the present disclosure, there is provided an annuloplasty device configured to deliver structurally disruptive energy to a device tip terminating a distal end of a catheter body, the annuloplasty device including: a transducer, configured to deliver structurally disruptive energy to a plurality of sites at positions around a circumference of a heart valve annulus; an elongated, steerable body, tipped by the transducer and including an elongated control member; wherein the control member is actuatable to coordinately curl the steerable body, and to bend the steerable body out of a plane of the curl.

Unless otherwise defined, all technical and/or scientific terms used herein have the same meaning as commonly understood by one of ordinary skill in the art to which the present disclosure pertains. Although methods and materials similar or equivalent to those described herein can be used in the practice or testing of embodiments of the present disclosure, exemplary methods and/or materials are described below. In case of conflict, the patent specification, including definitions, will control. In addition, the materials, methods, and examples are illustrative only and are not intended to be necessarily limiting.

As will be appreciated by one skilled in the art, aspects of the present disclosure may be embodied as a system, method or computer program product. Accordingly, aspects of the present disclosure may take the form of an entirely hardware embodiment, an entirely software embodiment (including firmware, resident software, microcode, etc.) or an embodiment combining software and hardware aspects that may all generally be referred to herein as a “circuit,” “module” or “system” (e.g., a method may be implemented using “computer circuitry”). Furthermore, some embodiments of the present disclosure may take the form of a computer program product embodied in one or more computer readable medium(s) having computer readable program code embodied thereon. Implementation of the method and/or system of some embodiments of the present disclosure can involve performing and/or completing selected tasks manually, automatically, or a combination thereof. Moreover, according to actual instrumentation and equipment of some embodiments of the method and/or system of the present disclosure, several selected tasks could be implemented by hardware, by software or by firmware and/or by a combination thereof, e.g., using an operating system.

For example, hardware for performing selected tasks according to some embodiments of the present disclosure could be implemented as a chip or a circuit. As software, selected tasks according to some embodiments of the present disclosure could be implemented as a plurality of software instructions being executed by a computer using any suitable operating system. In some embodiments of the present disclosure, one or more tasks performed in method and/or by system are performed by a data processor (also referred to herein as a “digital processor”, in reference to data processors which operate using groups of digital bits), such as a computing platform for executing a plurality of instructions. Instruction executing elements of the processor may comprise, for example, one or more microprocessor chips, ASICs, and/or FPGAs. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well. Any of these implementations are referred to herein more generally as instances of computer circuitry.

Any combination of one or more computer readable medium(s) may be utilized for some embodiments of the present disclosure. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device. A computer readable storage medium may also contain or store information for use by such a program, for example, data structured in the way it is recorded by the computer readable storage medium so that a computer program can access it as, for example, one or more tables, lists, arrays, data trees, and/or another data structure. Herein a computer readable storage medium which records data in a form retrievable as groups of digital bits is also referred to as a digital memory. It should be understood that a computer readable storage medium, in some embodiments, is optionally also used as a computer writable storage medium, in the case of a computer readable storage medium which is not read-only in nature, and/or in a read-only state.

Herein, a data processor is said to be “configured” to perform data processing actions insofar as it is coupled to a computer readable medium to receive instructions and/or data therefrom, process them, and/or store processing results in the same or another computer readable medium. The processing performed (optionally on the data) is specified by the instructions, with the effect that the processor operates according to the instructions. The act of processing may be referred to additionally or alternatively by one or more other terms; for example: comparing, estimating, determining, calculating, identifying, associating, storing, analyzing, selecting, and/or transforming. For example, in some embodiments, a digital processor receives instructions and data from a digital memory, processes the data according to the instructions, and/or stores processing results in the digital memory. In some embodiments, “providing” processing results comprises one or more of transmitting, storing and/or presenting processing results. Presenting optionally comprises showing on a display, indicating by sound, printing on a printout, or otherwise giving results in a form accessible to human sensory capabilities.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium and/or data used thereby may be transmitted using any appropriate medium, including but not limited to wireless, wireline, optical fiber cable, RF, etc., or any suitable combination of the foregoing.

Computer program code for carrying out operations for some embodiments of the present disclosure may be written in any combination of one or more programming languages, including an object oriented programming language such as Java, Smalltalk, C++ or the like and conventional procedural programming languages, such as the “C” programming language or similar programming languages. Additionally or alternatively, sequences of logical operations (optionally logical operations corresponding to computer instructions) may be embedded in the design of an ASIC and/or in the configuration of an FPGA device. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Some embodiments of the present disclosure may be described below with reference to flowchart illustrations and/or block diagrams of methods, apparatus (systems) and computer program products according to embodiments of the present disclosure. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

Some of the methods described herein are generally designed only for use by a computer, and may not be feasible or practical for performing purely manually, by a human expert. A human expert who wanted to manually perform similar tasks, such inspecting objects, might be expected to use completely different methods, e.g., making use of expert knowledge and/or the pattern recognition capabilities of the human brain, which would be vastly more efficient than manually going through the steps of the methods described herein.

### BRIEF DESCRIPTION OF THE SEVERAL VIEWS OF THE DRAWINGS

Some embodiments of the present disclosure are herein described, by way of example only, with reference to the accompanying drawings. With specific reference now to the drawings in detail, it is stressed that the particulars shown are by way of example, and for purposes of illustrative discussion of embodiments of the present disclosure. In this regard, the description taken with the drawings makes apparent to those skilled in the art how embodiments of the present disclosure may be practiced.

In the Drawings:

FIG. 1A is a schematic illustration of a heart valve annuloplasty device operable to shape valve ring tissue using vacuum, according to some embodiments of the present disclosure;

FIG. 1B is a schematic flowchart of a method of performing valve annuloplasty with the device of FIG. 1A, according to some embodiments of the present disclosure;

FIGS. 1C-1E schematically illustrate operation of the device of Figure IA, according to some embodiments of the present disclosure;

FIG. 1F is a schematic illustration of a lesion produced by the device of FIG. 1A, according to some embodiments of the present disclosure;

FIG. 2A is a photograph of a lesion produced ex vivo in porcine heart tricuspid valve using device, according to some embodiments of the present disclosure;

FIGS. 2B-2E are before-lesioning (FIGS. 2B, 2D) and after-lesioning (FIGS. 2C, 2E) photographs of regions of lesions produced ex vivo in porcine heart tricuspid valve using device, according to some embodiments of the present disclosure;

FIG. 2F is a photograph of a sectioned lesion produced ex vivo in porcine heart tricuspid valve using device, according to some embodiments of the present disclosure;

FIG. 2G schematically outlines regions corresponding to features of the photograph of FIG. 2F, according to some embodiments of the present disclosure;

FIGS. 2H-2I are photographs of rows of lesions produced ex vivo in porcine heart mitral valve using device, according to some embodiments of the present disclosure;

FIG. 2J is a photograph of a sectioned row of lesions produced ex vivo in porcine heart mitral valve using device, according to some embodiments of the present disclosure;

FIG. 3A is a schematic illustration of a distal end of a heart valve annuloplasty device, according to some embodiments of the present disclosure;

FIG. 3B is a schematic illustration of a distal-ward view of a tip of a heart valve annuloplasty device, according to some embodiments of the present disclosure;

FIG. 3C is a schematic illustration of a handle and connection region of a heart valve annuloplasty device, according to some embodiments of the present disclosure;

FIGS. 4A-4B schematically illustrate of a distal end of a heart valve annuloplasty device with the vacuum attachment element in advanced (FIG. 4A) and retracted (FIG. 4B) positions, according to some embodiments of the present disclosure;

FIG. 4C schematically illustrates a lesion produced by the device of FIGS. 4A-4B, according to some embodiments of the present disclosure;

FIG. 5A is a schematic illustration of a heart valve annuloplasty device operable to shape valve annulus tissue using needle penetration and torsion, according to some embodiments of the present disclosure;

FIG. 5B is a schematic flowchart of a method of performing valve annuloplasty with the device of FIG. 5A, according to some embodiments of the present disclosure;

FIGS. 5C-5F schematically illustrate operation of the device of FIG. 5A, according to some embodiments of the present disclosure;

FIG. 5G is a schematic illustration of a lesion produced by the device of FIG. 5A, according to some embodiments of the present disclosure;

FIGS. 6A-6B are before-lesioning (FIG. 6A) and after-lesioning (FIG. 6B) photographs of the region of a lesion produced ex vivo in porcine heart using device of FIG. 5A, according to some embodiments of the present disclosure;

FIGS. 6C-6D are photographs of the before—(FIG. 6C) and after—(FIG. 6D) sectioning appearance of the lesion of FIG. 6B, according to some embodiments of the present disclosure;

FIGS. 6E-6F are before-lesioning (FIG. 6E) and after-lesioning (FIG. 6F) photographs of the region of a lesion produced ex vivo in porcine heart using device of FIG. 5A, according to some embodiments of the present disclosure;

FIG. 6G is a photograph after sectioning of the lesion of FIG. 6F, according to some embodiments of the present disclosure;

FIG. 6H represents estimated motions of tissue and pin electrode positions superimposed on the lesion image of FIG. 6B, according to some embodiments of the present disclosure;

FIG. 6I represents estimated motions of FIG. 6B, according to some embodiments of the present disclosure;

FIGS. 7A-7B schematically illustrate a distal end of a heart valve annuloplasty device in different operating states, according to some embodiments of the present disclosure;

FIG. 7C schematically illustrates an end-on view of the operating states of FIG. 7B, according to some embodiments of the present disclosure;

FIG. 7D schematically illustrates a distal end of a heart valve annuloplasty device in different operating states, according to some embodiments of the present disclosure;

FIG. 7E schematically illustrates an end-on view of the operating states of FIG. 7D, according to some embodiments of the present disclosure;

FIGS. 8A-8B schematically illustrate a distal end of a heart valve annuloplasty device in different operating states, according to some embodiments of the present disclosure;

FIG. 9 is a photograph of the heart valve annuloplasty device of FIGS. 8A-8B in use during ex vivo lesioning of a porcine heart valve, according to some embodiments of the present disclosure;

FIGS. 10A-10C schematically illustrate a distal end of a heart valve annuloplasty device in different operating states, according to some embodiments of the present disclosure;

FIG. 11 schematically illustrates a distal end of a heart valve annuloplasty device, according to some embodiments of the present disclosure;

FIGS. 12A-12B schematically represent a casing of a tip of a valve annuloplasty device, according to some embodiments of the present disclosure;

FIG. 12C schematically represents a housing of a tip of a valve annuloplasty device, according to some embodiments of the present disclosure;

FIG. 13A schematically represents a partially assembled tip of a valve annuloplasty device, according to some embodiments of the present disclosure;

FIGS. 13B-13C schematically illustrates partially assembled tip of a valve annuloplasty device with housing added, according to some embodiments of the present disclosure;

FIG. 13D schematically illustrates tip of a valve annuloplasty device, now with catheter casing added, according to some embodiments of the present disclosure;

FIG. 14 presents a fluoroscopic (X-ray) image of a distal tip of a valve annuloplasty device in situ (that is, within a chamber of a heart), according to some embodiments of the present disclosure;

FIG. 15A schematically illustrates a horizontal (transverse) section through a left side of a heart including left atrium and left ventricle, according to some embodiments of the present disclosure;

FIG. 15B presents a 3-D reconstructed ultrasound image which looks down at valve and the horizontal (transverse) section from a vantage point above it, according to some embodiments of the present disclosure;

FIG. 15C schematically illustrates a horizontal (transverse) section through a left side of a heart including left atrium and left ventricle, according to some embodiments of the present disclosure;

FIG. 15D schematically illustrates a coronal (frontal plane) section through a left side of a heart including left atrium and left ventricle, according to some embodiments of the present disclosure;

FIG. 15E presents a planar ultrasound image providing a coronal cross-section (corresponding to coronal section) of valve (e.g., showing the valve leaflets), according to some embodiments of the present disclosure;

FIG. 15F schematically illustrates a coronal (frontal plane) section through a left side of a heart including left atrium and left ventricle, according to some embodiments of the present disclosure;

FIGS. 16A-16H present a sequence of 3-D reconstructed ultrasound images obtained at different positions and/or articulations of tip as it moves pin element around a circumference of mitral valve annulus, according to some embodiments of the present disclosure;

FIG. 16I overlays the various positions of FIGS. 16A-16H in a single image.

FIGS. 17A-17B illustrate fluoroscopic (X-ray) images in situ (that is, within a chamber of a heart) of a pin assembly (which may comprise a radiopaque material such as tantalum) in relation to radiopaque markers, as well as schematic drawings of the same, according to some embodiments of the present disclosure;

FIG. 18A schematically represents views of pin assembly and radiopaque markers at different rotational positions, according to some embodiments of the present disclosure;

FIGS. 18B-18D show X-ray images of a distal tip, including with views of pins from different rotational angles, according to some embodiments of the present disclosure;

FIGS. 19A-19B schematically represent an overtube predisposed to assume a compound bending which adds both curvature and displacement out-of-plane, according to some embodiments of the present disclosure;

FIGS. 19C-19D schematically represent an overtube (or a catheter itself, similarly configured for steering), in relation to a roughly saddle-shaped valve annulus, according to some embodiments of the present disclosure;

FIG. 20 is a schematic flowchart representing targeted and/or expected thresholds and/or ranges of bio-impedance in Ohms (Ω) for various phases and/or conditions during an annuloplasty procedure, according to some embodiments of the present disclosure; and

FIG. 21 is a schematic flowchart representing methods of operating a valve annuloplasty device, according to some embodiments of the present disclosure.

### DESCRIPTION OF SPECIFIC EMBODIMENTS OF THE INVENTION

The present invention, in some embodiments thereof, relates to the field of structural heart disease, and more particularly, but not exclusively, to heart valve annuloplasty.

### Overview

A broad aspect of some embodiments of the present disclosure relates to valve annuloplasty performed using tissue shrinkage and/or remodeling induced by energy applied to the region of the valve's annular ring. Examples of valves treated in some embodiments of the present disclosure include but are not limited to tricuspid heart valves and mitral heart valves.

Currently a gold standard of care to treat atrial fibrillation is the use of RF energy to ablate regions along the left atrial wall around the pulmonary veins. A reported side effect of this procedure is pulmonary vein stenosis (PVS). PVS may also be an outcome of ablation procedures performed using other methods such as cryoablation (e.g., as reported by J. Matilda et al., J Cardiovasc Electrophysiol. 2017 Mar;28(3):298-303. Pulmonary Vein Stenosis After Second-Generation Cryoballoon Ablation).

PVS is attributed to shrinkage of the pulmonary veins induced by shrinkage of ablated tissue areas. A physiological mechanism which creates stenosis in pulmonary veins due to ablation is attributed to scarring of connective tissue surrounding the pulmonary veins, for example as described in Pulmonary Vein Stenosis After Catheter Ablation, Electroporation Versus Radiofrequency by Vincent JAM et al., Circ Arrhythm Electrophysiol. 2014 Aug; 7(4):734-8.

The inventors describe herein an endovascular approach using the phenomenon of tissue shrinkage induced by applying structurally disruptive energy to treat heart valve leakage. Leakage is characterized by failure of the heart valve's leaflets to coapt—they do not close fully in response to back-pressure. This allows blood flow regurgitation, and impairs the efficiency of pumping by the heart.

In some embodiments of the present disclosure, tissue on the perimeter of the valve annulus is remodeled by the application of structurally disruptive energy in combination with the application of mechanical forces which deform the tissue.

In some embodiments, this comprises energy sufficient to induce tissue fibrosis. In some embodiments, this energy is delivered in the form of radio frequency (RF) electromagnetic power, which produces localized heating of tissue, e.g., to the extent that fibrosis is induced. In some embodiments, another form of energy delivery producing heating is used. It is a potential advantage for that energy to be provided in a form which converts to produce a significant level of heating only upon delivery to a target site, and/or a form that can be focused to a target without resulting in significant heating effects outside a region of focus. For example, acoustic energy (e.g., focused ultrasound) or laser energy is used. The effect on tissue of the application of structurally disruptive energy is also referred to herein as “lesioning”. Such a treatment may also be referred to as an “ablation”, e.g., insofar is it produces cellular death in a portion of the treated tissue. The delivery of energy may lesion the valve annulus tissue directly (that is, lesion collagenous tissue of the valve annulus), and/or tissue nearby; for example, the atrial wall above the mitral or tricuspid valve.

Some embodiments of the present disclosure are configured and operated to impose mechanical forces on treatment sites to which structurally disruptive energy is also applied. In brief, the tissue is mechanically deformed (e.g., compressed, but not necessarily only compressed) in the region of energy delivery, deforming its shape. Structure-disrupting energy is applied-and upon release, at least a portion of the mechanical deformation remains imprinted on the tissue-a plastic deformation.

Suitably placed and optionally repeated at a plurality of sites, this potentially brings leaflets of a regurgitating heart valve into coaptation; or if coaptation is not achieved, may reduce the severity of the regurgitation by reducing the remaining gap between them in their most-closed state. In some cases, an original loss of normal valve leaflet coaptation was itself caused by reshaping (lengthening) the valve annulus. Accordingly, a treatment that shrinks the valve annulus may return the heart valve leaflets into their original relationship with one another.

The plastic deformation effect does not necessarily require cellular death (or at least, not complete cellular death) to be induced by the application of structurally disruptive energy. For example, at a sub-ablation threshold, the fibrotic structure of tissue may be made more malleable by its heating, and/or by changes in its pH due to the passage of an electrolyzing current. The structural disruption which produces this malleability is optionally induced concurrently with or separately from (in time) the mechanical manipulation. However, the period of plastic malleability may be so short as to practically require mechanical deformation to be first induced and then held while the structurally disruptive energy is applied.

Effects of plastic remodeling under mechanical force described for embodiments herein are generally acutely apparent; that is, they occur during the application of structurally disruptive energy or within a brief period thereafter, while a procedure is underway that uses a tool to apply the mechanical force.

Without commitment to a particular theory, these acutely apparent effects may be understood as influenced by the disorganizing effects of coagulation acting to relieve stresses and/or strains in tissue deformed by external mechanical forces. This effectively gives the tissue a new “preferred shape” in its coagulated state, even after the external mechanical forces are removed (i.e., it is plastically deformed). It is not excluded that there may be non-coagulating mechanisms influencing plastic deformation when applying structurally disruptive energy to tissue deformed by external forces. For example, a mechanism has been proposed by which electrolysis of water in a tissue region results in the production of free protons which in turn temporarily affect self-binding of the collagen matrix so that it become more malleable.

Additionally to the above, the disruption by structurally disruptive energy may itself induce shrinkage-and a corresponding reduction in the overall valve annulus perimeter. Without commitment to a particular theory, shrinking may result, for example, from loss of cellular structures, from relaxation of internal stresses on connective fibers, from effects of denaturation (coagulation) on tissue structures that persist after energy delivery, and/or from effects of healing processes which occur post-treatment.

Shrinkage may comprise effects which occur immediately or almost immediately (e.g., due to losses of fluid or shrinkage of cellular components), and slower effects due, e.g., to induced atrophy and/or processes of healing.

Two main types of ablation performed on heart tissue for treating atrial fibrillation are thermal ablation, e.g., using radiofrequency (RF) energy or ultrasound energy; and cryoablation. Both types of ablation have been associated with pulmonary vein stenosis. However, there may be differences in tissue remodeling effects as a result of differences in the two mechanisms. For example, thermal ablation effects include coagulation acting directly on structural cellular components, while cryoablation's main effects disrupt cellular organization and processes leading to downstream degeneration of structural cellular components, potentially under biological control. Electroporation is another cellular ablation mechanism which is primarily disruptive in its initial effects rather than denaturing (coagulating). As mentioned above, electrolysis of tissue water has also been proposed as a mechanism for disrupting the collagen matrix by partially acidifying it. In some embodiments, another mechanism for applying structurally disruptive energy is provided; for example, acoustic energy (e.g., focused ultrasound) or laser energy. It is not excluded that direct application of heat energy is used, e.g., via electrical heating of a heating element in contact with tissue to be remodeled. However, modes of applying energy which include penetration of tissue before conversion to heat provide potential advantages for evenness of energy distribution, avoidance of hot spots which (e.g., charring or other “overcooking”), and/or safety. In some embodiments of the present disclosure, annuloplasty performed by structural disruption of tissue reduces overall valve perimeter by up to about 5-10%, or potentially a larger amount. Reshaping of the valve annulus may be targeted to sites at any selected portion of the valve annulus perimeter; for example, disrupting tissue at approximately evenly spaced locations, or alternatively at locations grouped in one or more particular regions around the perimeter. Tissue involved in individual lesions may be reduced in size considerably more than 5-10%. For example, ex vivo experiments described herein were found to induce shrinkage in focally treated areas about 5 mm across by a factor of more than 20%, up to about 40%. These values may not represent upper limits.

An aspect of some embodiments of the present disclosure relates to annuloplasty devices which couple the use of structurally disruptive energy coupled to mechanical manipulation by torsion.

It may be appreciated, for example, from teachings in International Patent Publication No. WO2002/097130, that torsion applied to tissue may provide a mode of applying mechanical deformations to tissue that may be plastically set by the application of structurally disruptive energy.

In further studies, the inventors have found that for a same overall device diameter, use of torsionally induced mechanical shrinkage potentially produces greater shrinkage than pinching-type compression, wherein pins move toward each other. For example, shrinkages per lesion location induced to an average of 1.9 mm using a pinch compression device may be increased with a torsion-inducing device up to about 2.5 mm.

In some embodiments of the present disclosure, the torsion is exerted through rotation of two separately penetrating manipulation elements around a common center. Herein, such a manipulation element is also referred to as a pin (more particularly the term “pin electrode” may also apply, when the functions of both electrode and mechanical manipulation are shared by a same element).

Compared to a direct pin-toward-pin compression movement (pinching), for example, torsion provides a potential advantage of exerting mechanical compression upon tissue which is beyond the pins, potentially including beyond the pins and roughly along and beside an axis extending between the two pins. Compression directly between the two pins, conversely, may be somewhat lessened (e.g., insofar as they do not become closer to one another). The different pattern of tissue shrinkage may result in a more even distribution of internal forces, which may help reduce a potential for tearing during application of structurally disruptive energy, and/or may help reduce unwanted distortions in the treated valve annulus. Greater distribution of shrinkage may also lower the peak amount of deformation introduced, so that the end result is a relatively smoother surface for similar, the same, or greater amounts of shrinkage introduced. A single large central pinched-up region, for example, may be replaced by a lower central bulge, with additional peripheral wrinkling as needed to further accommodate the deformations introduced.

Furthermore, there may be limits to how much any given surface of tissue can be shrunk, so there is a potential advantage in a mechanical method of deformation that distributes shrinking over a larger area. Valve annulus shrinkage may preferably be focused in areas near where valve leaflets should coapt. Thus, there is potentially a premium on being able to achieve relatively large amounts of shrinkage within a single lesion. Insofar as the circumferential space available in preferred placement location(s) is limited, each treatment site there may preferably receive as much shrinkage as can be reliably achieved. In any case, this may allow fewer treatment sites overall to be accessed, potentially simplifying and/or shortening annuloplasty procedures. This may also make it easier to design procedures which avoid going too near to delicate and/or functionally critical areas of tissue like the AV node (e.g., in the case of a tricuspid valve annuloplasty procedure), by focusing shrinkage to regions away from such delicate and/or functionally critical areas.

Torsional distortion of tissue may allow a smaller device footprint to be used to achieve the same distance of effective shrinkage per treatment location. For example, instead of shrinking a footprint of about 4 mm initial width of contact by about 2 mm (e.g., by pinching), using rotational motion (torsion) potentially allows a fixed 1.5 mm initial width footprint to achieve the same amount of effective shrinkage. This potentially allows placement of more treatment locations within a single procedure, to achieve greater overall shrinkage. It potentially allows more concentrated (linearly denser) placement of treatment locations, to focus valve annulus remodeling where it is thought most likely to promote coaptation and/or to avoid areas of risk such as regions near an AV node or other sensitive region. Furthermore, it potentially leaves more unmodified tissue available for modification in future procedures, e.g., should the valve continue to reshape over time, and/or should it be found that an initial procedure did not achieve full or intended results. This also potentially allows a physician freedom to be less aggressive in a first annuloplasty procedure, insofar as options for a second annuloplasty procedure, if necessary, are left more open.

With a pinching/squeezing approach, it is compression and/or displacement of tissue within the initial “bite” distance of the manipulating elements which determines how much shrinkage can occur. The “bite” distance sets a first order maximum on per-site shrinkage; but practically the tissue is not expected to shrink or displace enough to allow the biting “jaws” (e.g., needles, pins, or other gripping and/or penetrating element) to completely converge. As a result, the maximum shrinkage may be much less than the initial bite distance.

Using torsional distortion, however, tissue regions moved in opposite directions are also offset from each other, so that they do not also move directly toward each other (in the direction of a collision). Accordingly, a 90° rotation, for example, may result in some distances along at least some axis (e.g., a distance between two tissue regions in a circumferential direction around the valve annulus) being shortened by about the full distance between pins. Not all regions will be so affected; e.g., torsion also performs the complementary transformation on other tissue (e.g., displacing previously “vertical” arrangements of tissue to be more “horizontal”; that is, more distant along the circumference of the valve annulus). The overall pattern of distortion of tissue by torsion may, however, be biased away from complete radial symmetry (e.g., toward shrinkage mostly along the valve annulus circumference, and less along the valve annulus height) by suitable initial placement of the manipulating elements and selection of their angle of rotation.

Moreover, since torsion tends to draw tissue radially inward overall, it also induces a component of tissue compression. That compression may be lower in terms of, e.g., peak percentage than compression placed on “pinched” tissue, but it may also be distributed over a larger area (e.g., including compression of areas in line with but beyond the diameter of the “bite” established by the manipulating elements), so as to potentially result in a similar or larger net effect on circumferential size than squeezing would achieve with the same “bite” diameter.

Considering a different optional priority, it may be a potential advantage to distribute valve annulus shrinkage as evenly as possible-for example, to reduce the level of intervention in any one location, and/or to avoid introducing irregularity into valve annulus shape or function. In that case, the emphasis may be shifted to evenness of treatment effects, and, correspondingly, to the planned control of how much shrinkage occurs in each location.

In some embodiments of the present disclosure, there is provided an annuloplasty device configured to exert torsional distortion through a limited range of motion provided to pins, the motion being limited by the device to a predetermined maximum angle or otherwise specified maximum distance. The limitation may be absolute, or it may be dependent on operating the device in an intended mode which is self-limiting. In some embodiments, the full range of motion available is also the normally preferred or user-selected amount used to gather tissue. In some embodiments, the range of motion is selectable, for example by choice of which specific annuloplasty device is used, and/or by adjustment of a setting (e.g., a slot size, a protrusion or other interference element, or range of control motion) which imposes desired limits on the range of motion. Partial rotation within the limited range available is also an option in some embodiments, with the limits on rotation acting, e.g., as a safety limit.

Rotational limits may be set, for example, by stops comprising structures which physically interfere with each other to prevent further motion upon coming in contact. Such stop arrangements may be provided at the tip of the device (e.g., to directly prevent rotation beyond a certain angle), and/or provided to limit movements of control elements. Additionally or alternatively, sensing may be provided to indicate rotational position, e.g., using electrical contacts or another form of electronic and/or mechanically implemented position encoding. Sensing state may be indicated to a user by means of LEDs, haptic vibrators, buzzers and/or speakers, indications presented on a screen, or another method.

Providing for ensuring a predetermined range of rotational motion has potential advantages for the exertion of torsion in a manner which is sufficient to achieve shrinkage-but not excessive, so as to result in tearing or other unintended damage at the time of force application, and/or upon the delivery of structurally disruptive energy. For example, the application of heating through RF energy delivery has the potential to weaken tissue to an extent that may result in induction of a tear in tissue under excessive stress. Thus, there may be a preferred (e.g., validated) maximum amount of rotation which can be safely tolerated.

Moreover-and also relevant to above type of problem-the predetermined range of rotational motion helps torsion be applied in a manner which is predictable and/or reproducible. A surgical plan may target a particular amount of valve circumference reduction and/or other remodeling, e.g., using a particular number of treatment sites. It is a potential advantage to be able to use the annuloplasty device to set (or select) shrinkage targets which it itself helps to ensure.

In some embodiments of the present disclosure, there is provided an annuloplasty device configured to exert torsional distortion by rotation of two separately penetrating manipulation elements around a common center positioned medially between them. Additionally or alternatively, in some embodiments of the present disclosure, there is provided an annuloplasty device configured to exert torsional distortion by rotation of two separately penetrating manipulation elements around a common center positioned nearer to one of them. Optionally the common center is also along an axis of one of the penetrating manipulation elements. In some embodiments, torsion is produced along with direct compression exerted by pins coming nearer to each other. Optionally, the direct compression is transient (e.g., two pins slide past each other, but end up at their original distance from each other).

Although differences in axis of rotation offset potentially somewhat change how force is exerted through tissue as the pins rotate, redistribution of forces may end up displacing the effective axis (if not otherwise fixed in place) to the same final location, so that the differences produced are minor, if any.

Variations in the orientation of another axis, centered on and extending through both pins when they initially penetrate into tissue, is potentially associated with significant differences in the resulting pattern of tissue deformation. Approximately described: tissue in surface regions angularly nearer to the above-defined initial penetration axis may tend to be drawn more relatively more around its center (i.e., tangentially), and tissue in surface regions far from it may tend to be drawn more toward the center (i.e., radially). Thus, depending on how the initial axis of penetration is oriented, along one axis (e.g., along the valve annulus circumference), all four quadrants have a significant component of shrinkage, while along the orthogonal axis (e.g., along the height axis of the valve annulus), only two of them do. Stated otherwise, there is a “shrinkage ratio” between the two axes which is not necessarily equal to 1, also referred to herein as shrinkage anisotropy. The actual shrinkage anisotropy may depend on the amount of angular twist given to the pins, and may be particularly relevant for rotations significantly less than a full rotation (e.g., less than 180° or less than 90°). In some embodiments, the pins are rotatable by at least 45° while inserted to tissue. The pins are anchored on a proximal side to provide sufficient rigidity to overcome forces in the tissue which tend to resist deformation. For example, they are anchored to a common base which rotates within a housing, or to respective bases which move by rotation or otherwise relative to the housing. In some embodiments, one of the pins is anchored to a housing, while the base anchoring the other is moveable relative to the housing.

It should also be noted that for a large (and potentially the largest) shrinkage in a selected direction (e.g., between two points A and B), the initial axis of penetration may be at an angle to a mid-plane perpendicular to and bisecting AB, such that the mid-plane bisects the angle through which the pins establishing the penetration angle sweep from their initial position to their final position. For example, if the sweep angle is about 90°, the initial penetration axis is at about a 45° angle from the bisecting mid-plane. To target maximal shrinkage generally around the circumference of a valve annulus, the mid-plane may be selected as extending parallel to the height axis of the valve (e.g., an axis extending from a first side of the valve annulus to a second side of the valve annulus, and perpendicular to a plane with maximally uniform distance from the circumference of the valve annulus). Noting expected variation in anatomy and/or carrying out these instructions, said mid-plane is preferably within 15° of the actual bisecting angle, or another angle, for example, selected as 10°, 20°, or 25°.

Accordingly, in some embodiments, torsional deformation comprises rotating an axis extending between tissue-embedded pins of the device from a first angle oblique to the local circumferential direction to a second such angle, through an intermediate angle which is perpendicular to the local circumferential direction. In some embodiments, the first and second angles are offset from the intermediate angle about equally, e.g., within 15° of each other. In some embodiments, the first and second angles differ from each other by about 60°-120°.

For purposes of resolving disagreement in competing applications of the foregoing instructions, circumference definitions more nearly approximating a primary valve circumference in result (if comparison is available) or definition (otherwise) are preferred. A primary valve circumference extends in a closed curve along and around a superficial surface of the collagenous tissue of the valve annulus. It is positioned at equal distances between edges of the collagenous tissue. For purposes of practical calculation, edge distances are measured only for a few clearly discerned landmark locations around the circumference, preferably 8 locations, preferably evenly spaced, preferably with one location anchored at the widest part of the valve annulus. Positions in-between are smoothly interpolated by spline fitting. Should selection of a particular heartbeat phase be needed to resolve the definition of a circumference to sufficient precision, a phase with the heart valve annulus more nearly approaching a planar shape is more preferred. These considerations optionally apply generally for purposes of resolving disagreements hinging on geometrical particulars of valve circumference.

There are potential advantages for annuloplasty results in providing control of shrinkage anisotropy. Potentially distinct from “pinch” methods of introducing mechanical deformation, torsion-induced deformation is omnidirectional in its effects; tissue tends to be drawn at least partially inward from all sides. This may result in torsion inducing shrinkage along the valve annulus height axis which is not necessarily in itself therapeutically meaningful, although it may also be a byproduct of potential benefits (e.g., with respect to pinch-type deformation) in shrinkage evenness, shrinkage distribution, and/or stress relief. Accordingly, in some embodiments of the present disclosure, there are provided controls and methods of operation which assist in managing how shrinkage anisotropy is introduced to the target tissue.

In some embodiments of the present disclosure, the pins which rotate are constructed to rotate in response to actuation of a control on a distal side of an elongated element (e.g., an elongated tubular element, also referred to herein as a catheter) to which they are attached. The control optionally comprises, for example, a button, a knob, a slider, a lever, or another mechanism. The control optionally operates, for example as a release (e.g., of spring-loaded tension). Optionally, the control rotates the pins through an angle having a magnitude corresponding to a degree of movement imparted to the control. The rotating pins are configured to rotate while embedded in valve annulus tissue so that the valve annulus tissue surrounding them moves along with them. Accordingly, control exerted on them is configured to transmit an amount of torque significantly larger than would be needed to rotate the pins in free fluid, for example at least 0.002 N·m, 0.005 N·m, 0.01 N·m, 0.02 N·m, or another amount of torque. In some embodiments, the twisting is sufficient to deform tissue according to one or more of various criteria. For example, pin rotation moves some tissue relative to one of the pins (a reference pin) by at least 90% of the distance moved relative to the reference pin (e.g., between two points on a circumference around the reference pin) by another of the pins (the moving pin, in the given frame of reference). The tissue may be the tissue immediately in contact with the moving pin, or other tissue, for example, tissue which is at least 25%, 50%, 75%, or 100% of the inter-pin distance away from the moving pin. This criterion uses the reference pin to define a frame of rotational reference for purposes of measurement; the actual rotation imparted (i.e., relative to a frame of reference comprising fixed objects in the distance) is not necessarily of one pin around another; it could even be of the reference pin around the nominally “moving” pin. This criterion may be modified to include a minimum threshold of relative angular movement of the pins, for example, at least 30°, at least 45°, at least 60°, or at least 90°. When the pins also change their relative distance, the same criterion may also be applied, modified as necessary to account for relative linear motions.

To maintain their stability during rotation, the pins are preferably short, though long enough to be compatible with their role in manipulating tissue. For example, the pins are about 2-6 mm long, 2-8 mm long, or another length. Furthermore, the pins are secured stably (e.g., mounted to a mounting block) so that they can transmit manipulation torque to the tissue rather than themselves deforming, e.g., without flexing by more than 10° or so, and preferably flexing less than this. The pins may be mounted, for example, with their axial centers about 1-2 mm apart, e.g., about 1.3 mm apart. This range of distances is suitable, for example, for use with pins having maximum diameter of about 0.6 mm (e.g., 0.4 mm×0.4 mm square-cross section pins), and a whole catheter having a maximum outer diameter of about 5 mm. In some embodiments, the axial centers of the pins are mounted apart by a larger distance, e.g., about 3 mm apart, about 4 mm apart, or another distance. The distance is preferably not larger than the axial height of the valve annulus (e.g., the circumferentially continuous ring of collagenous tissue). In some embodiments, the distance is less than 80% of this height, less than 66%, less than 50%, or another maximum relative distance. Large distances may tend to deform the valve annulus along the axial height axis to an unacceptable degree, though they may potentially allow fewer lesions to achieve a targeted degree of shrinkage. However, such distortions may be acceptable and manageable in some situations, for example by alternately distorting clockwise and counterclockwise. Large distortions in a same direction may even be desirable in some cases, e.g., to restore the valve shape from a particularly deformed annular shape.

In some embodiments, the pins are alternately extendable and retractable along a proximal-distal axis of their housing (that is, the housing can be withdrawn/advanced, and/or the pins can be advanced/retracted). In the retracted state, the pins can be moved through the body without catching on unintended structures. For example, during positioning, they can be moved into contact with the valve annulus ring while retracted, avoiding a risk of nicking and/or entangling with the valve leaflets. Extended (e.g., after placing a proximal end of the housing which contains them against the valve annulus tissue to be treated), the pins are operable to engage with valve annulus ring tissue.

In some embodiments, the housing comprises a distal portion of an elongated tubular element (also referred to herein as a catheter), and/or a separate element attached to a distal end of the catheter. In some embodiments, the housing comprises a portion of an overtube used to introduce the pins into a region. Optionally, the housing is specialized for the functions of the annuloplasty device tip. For example, the distal portion is optionally shaped and/or sized differently in cross-section than the catheter it extends distally from, accommodating elements of one or more mechanisms actuating movement of the pins, and/or the pins themselves. In some embodiments, the housing is provided with echogenic structures such as grooves, cross-hatching, or another surface texture, which potentially enhances visualization of its position under echocardiography. In some embodiments, the housing is provided with radiopaque structure (e.g., made of tantalum, gold, and/or tungsten) to enhance its visualization. In some embodiments, at least one of the pins is moveable relative to the housing, in a manner that applies torsion to tissue when pins are inserted to that tissue.

In some embodiments, actuation of pin rotation is controlled so that it occurs by a predetermined amount per corresponding amount of control movement and/or force exerted. This is a potential advantage for providing feedback (“feel”) to the device operator that allows them to judge the state of the tip, e.g., whether it is exerting sufficient resistance to indicate correct insertion into tissue, that it is not experiencing losses of resistance that may indicate tearing and/or loss of engagement, and/or whether it is exerting excessive resistance indicative of jamming or insertion into unexpectedly resistant (e.g., already treated, previously fibrotic, and/or calcifying tissue).

Actuation may comprise, e.g., linear movement of a long thin control member (e.g., a wire or string) being converted at the tip to rotational motion; for example, by spooling or unspooling from the pin mounting to rotate it, optionally against a restorative force such as a tensioned spring. In some embodiments, the control member is also a member along which structurally disruptive energy is transmitted to the tip, e.g., an electrically conductive wire.

In some embodiments, the control member actuates gearing or pulleys arranged to provide mechanical advantage in order to rotate the pin mounting. In some embodiments, a control member actuates rotation by rotating itself (around its longitudinal axis), and is sufficiently stiff in torsion and/or reproducible in effect (e.g., by being pre-loaded until it overcomes rotational resistance at least in one rotational direction) that the angle of pin rotation is predictable under the resistance forces which tissue-inserted pins are expected to absorb from valve annulus tissue.

In some embodiments, the linkages that actuate rotation are elastic and/or flexible (rotational and/or longitudinally) to the extent that actual rotation of the tissue-embedded pins achieved per unit of control actuation delivered is variable and/or non-linear. The actuation force, accordingly, may be built up as the flexible linkages partially absorb (by deforming) and partially transmit force, until enough force is transmitted to the pins through their stable mounting to rotate them. Rotation of the pins in such cases may be accomplished little by little as the control is moved, and/or may be accomplished all at once, for example by a triggered release of energy already built up in the device; for example, energy stored in a spring.

Whether under predictable control or under non-linear/poorly reproducing control, determination of the final amount of twisting may be achieved by limiting the range of rotational motion available, e.g., as described hereinabove. Providing hard limits to rotation has the potential advantage that non-linearities and/or reproducibility errors in actual compared to commanded motions may be unimportant during operation, once the full range of motion has been achieved. Partial rotations through the available range of motion may induce smaller amounts of shrinkage per lesion introduced, which may be suitable according to the design of the particular treatment procedure.

At least two further types of control may be distinguished, which may be operated to control the directionality of tissue manipulations introduced by torsion. First, in some embodiments of the present disclosure, the device may be steered in such a way as to maintain control of the orientation. For example, the device may be advanced along a height axis of the valve (e.g., near the valve center, though not necessarily near the center), and bent away from the axis toward the valve periphery. Upon rotating the bent device (including steering sheath or other steering mechanism) around the height axis, the alignment of the distal face of the device tip will retain its initial orientation relative to the valve height axis no matter where it turns. If, furthermore, the tip is rotated around its longitudinal (proximal-to-distal axis) the distal face of the device tip will change its rotation relative to the valve height axis. Together with suitable indications provided to indicate current relative rotational states of the steering sheath and the annuloplasty device it steers, the orientation of the tip can be known and controlled. The indications may comprise, e.g., marking on a control of a handle, radiopaque markings, echogenic features, détentes, and/or an optical or electronic encoder.

Second, in some embodiments of the present disclosure, the degree of twist imparted (before insertion of the pins, and/or after) may affect the direction and/or magnitude of anisotropies, as well as the overall amount of tissue distortion exerted. For example, rotation of the pins may be partially actuated before they are inserted into tissue, in order to change the shrinkage anisotropy orientation. This may comprise operation of a separate control than that used to actuate pin rotation during tissue manipulation, or it may be the same control. For example, rotation of the overall catheter body may be used to change the pre-insertion pin orientation, and another control used for post-insertion pin rotation relative to the catheter body. Furthermore, delivering a relatively larger or smaller twist from the starting orientation may affect the level of shrinkage anisotropy (although in this case, the overall amount of shrinkage induced mechanically may also be affected). Features and methods related to controlling angles of device twisting are described above.

To explain the context of anisotropy control in more detail: a targeted outcome of annuloplasty is shrinkage of the circumference of the treated valve annulus (or valve ring), perhaps by only a few percent (e.g., 5-10%). The normal circumference may be, for example, about 8-9 cm in the case of a human mitral valve; or about 12-14 cm in the case of a human tricuspid valve. However, the collagenous (fibrous) tissue of the valve annulus also has an axial height of only a few millimeters (e.g., less than 10 mm) . Thus any shrinkage induced may be a far greater fraction of the axial height than of the circumference (on average, or a large portion thereof) which is actually targeted. Accordingly, it is a potential advantage to focus an axis of maximum shrinkage along the circumference, while imposing relatively lower shrinkage along the axial height of the valve annulus.

However, rotating to shrinkage anisotropy to a different orientation may nevertheless be helpful in some cases, e.g., to even out shrinkage and help avoid introducing shape irregularities to the valve annulus. For example, directed control of the axis of shrinkage anisotropy potentially helps avoid or reduce the creation and/or aggravation of valve shape irregularities. Examples potentially include sharp variations in valve annulus height, irregularity of valve annulus axial position (e.g., induced valve annulus bending), reduction in flexibility, and/or modification of flexibility that functionally interferes with valve annulus shape dynamics.

During a heartbeat, a valve annulus may flex, for example, between a relatively flattened form, and a relatively saddle-shaped form. It may be preferable, in an annuloplasty procedure, to induce shrinking along the hinging regions of the saddle (regions where the annulus reverses its bend) in a manner which is different than in other locations, so as to encourage (or at least, avoid interfering with) flexing in correct directions. For example, areas with shrinkage preferentially induced along the height axis of the valve (compared to the local circumferential axis) may have weaker resistance to bending that occurs in normal valve annulus dynamics.

It is noted that a normal valve annulus is capable of significant dynamic remodeling (although this may be impaired already by the time annuloplasty is needed). It may be desirable, where feasible, to leave as much residual tissue flexibility intact as is compatible with the goal of promoting valve coaptation. Conversely, it may be an aim, in some uses and/or embodiments of the present disclosure, to introduce a curvature or kink to the valve annulus. For example, this may be aimed at introducing an opposite curvature or kink which is not addressed (or not fully addressed) by circumferential size reduction alone. In such cases, control of shrinkage anisotropy may provide a surgeon with a tool to assist in valve annulus sculpting. It should be noted that shrinkage anisotropy may not be only measured as relative shrinkage along orthogonal cross-axes; it may also apply to directionality of deformation (e.g., up or down) along a single axis; for example, induced by offsetting a location of a lesion. It may, for example, be preferable to pull axially superior tissue toward an axially inferior position, or alternatively, the opposite.

For purposes of illustration, and without commitment to a particular model of tissue deformation under torsional forces, an understanding of results produced by torsional shrinkage of valve annulus tissue may be assisted by the following descriptions of torsional effects on tissue. Torsional patterns of shrinkage tend to include a certain amount of pulling in of tissue from all or nearly all radial directions, as some component of the forces generated converts to tangential stress which tends to pull regions more distance than the tangent circle at least partially inward. However, when rotation is significantly smaller than a full rotation, for example, less than half a rotation (and nearer to a quarter of a rotation is more typical, in some embodiments of the present disclosure), the pattern of shrinkage tends to be dominated by movements along the initial direction(s) of motion, particularly in regions that can be directly pulled in that direction by one of the moving pins. Comparing this movement in tension to movements on the side of the pin where tissue is most directly pushed, the tissue may be more amenable to shrinking its flat surface equivalent area under compression than it is to expanding it under tension. The shrinking of the flat surface equivalent area can be accommodated by bulging and/or wrinkling even if the actual surface and/or volume hardly changes. But expansion under tension is limited by the elasticity of the fibrous tissue. Insofar as such elasticity exists, it is potentially relatively low compared to the capacity of the tissue to laterally compress by bulging/wrinkling

In some embodiments, more than two pins are used, for example, three, four, five, or more pins. Whether two pins or more are provided, any number of the pins may additionally be operable as electrodes through which ablation energy is delivered, and/or as sensing electrodes (e.g., sensing of impedance to help detect insertion and/or tissue state status). With larger numbers of pins than two, the pattern of distortions introduced may tend to be less anisotropic. For example, a four-fold distortion pattern (for four pins) repeats every 90° instead of every 180° (for a two-fold distortion pattern). There may be lowered peak forces focused on individual pins. If more than two pins are also used as electrodes, there may be a reduction in peak heating focused on individual pins (for the same total energy and/or power). Using two pins has potential advantages, e.g., for focusing insertion force on a smaller surface area, and/or for packaging of the device with a smaller delivery size.

There is no particular limitation that all pins must be of the same geometry. For example, one pin may be of small radius/maximum dimension, while a second pin may be of larger radius/maximum dimension. Pins are optionally square in cross section (e.g., 0.4 mm×0.4 mm in cross-section), or have another cross-sectional shape; for example, they may be round, triangular, or another shape. Flat-sided cross-sections (e.g., suitably oriented so that a flat surface faces the circumferential direction of rotation) provide potential advantages for providing a uniform surface against which torsion forces are distributed. Round cross-sections provide potential advantages for avoiding concentrations of ablation energy, when pins are also used as electrodes. Pins are optionally of uniform or non-uniform cross-section. Regions of non-uniform cross section may provide an advantage for penetration, e.g., tapers to provide sharpened tips of pins. In some embodiments, pin tapers may be relatively short (e.g., less than a distal 25%, 20%, or 10% of the overall pin length). Optionally, pin tapers are longer (e.g., 30%, 40%, 50%, or more of the overall pin length). This potentially allows the base of the pin to be significantly wider while still achieving sufficiently easy penetration. This in turn provides a potential advantage for redistributing contact surface area around a larger pin circumference to avoid tearing as it exerts force on tissue, either during rotation to deform tissue, or during administration of structurally disruptive energy. It may also strengthen the pin itself. The taper is not necessarily radially isotropic, e.g., the taper may be from a first cross-sectional shape to a second cross-sectional shape which is not merely scaled from the first cross-sectional shape.

There is no particular limitation that all or any pins be straight; for example, they may be curved (e.g., as hooks). In some embodiments, curved pins advance into tissue along paths which follow their curvature, or there may be some skew between pin shape and the pathway of pin advance. This potentially produces some deformation simply as a result of pin penetration (e.g., pinching), although there may be a concomitant increase in resistance to pin penetration.

Even for straight pins, there is no particular limitation that any or all pins advance into tissue while moving parallel to a longitudinal (proximal-to-distal) axis of the device which brings them against tissue. For example, pins may advance obliquely into tissue, optionally also in different directions from each other so that the pin tips converge or diverge as they advance.

Mutually non-parallel paths of pin/hook advance provide potential advantages for retaining penetrating attachment during further tissue manipulation, for example, force may be applied to pull tissue on tissue, e.g., pull the tissue against superficial electrode surfaces, for example, as also explained in relation to some embodiments using vacuum attachment to tissue, e.g., hereinbelow. An aspect of some embodiments of the present disclosure relates to annuloplasty devices which couple the use of structurally disruptive energy coupled to mechanical manipulation using vacuum.

In some embodiments, mechanical shrinkage of tissue before application of structurally disruptive energy is achieved by vacuum fixation of the tissue surface. A vacuum aperture is placed against the tissue in the region to be shrunk, and vacuum activated. In some embodiments, the resulting drop in pressure is at least, for example, about 50 mm Hg, 75 mm Hg, 100 mm Hg, 150 mm Hg, or another pressure value.

Tissue is drawn up into the aperture as a result. As this happens, there is concurrently an effective shrinkage of the flat surface equivalent area of the drawn-in tissue. For example, for a flat surface equivalent area defined as the cross-sectional area of the vacuum aperture, the actual surface area is that large plus the area which is needed to allow the tissue to bulge inward into the low-pressure vacuum aperture. Upon application of structurally disruptive energy the deformation becomes fixed, and the tissue is shrunk. In some embodiments, the structurally disruptive energy is delivered in the form of radio frequency (RF) energy, for example, with a power of 8-12 Watts over a time of from 12-24 seconds. In some embodiments, 8 Watts of power are

A potential advantage of this method of mechanical fixation is that fixation can be confirmed by vacuum pressure and/or evacuation volume changes. For example, in some embodiments, a pressure sensor is placed (e.g., in a tip of the device) where the stability of vacuum engagement can be determined from the surrounding pressure.

In some embodiments, the vacuum aperture is provided with an oblong shape. This potentially increases the fractional shrinkage of tissue in the shorter direction of the vacuum aperture. For example, approximating the bulge shape as a hemisphere split in the middle by a straight section, the ratio of actual surface length to effective flat surface length across the straight section's short distance d₁ may be, for example, about

$$\frac{π}{2} .$$

In the orthogonal direction, surface along the longest distance d₂ may be increased by this ratio only considering the two half hemispherical ends, with the ratio of the straight section in between being unchanged, so as to reduce the overall ratio. In some embodiments, the ration of height (long axis) to width (short axis) of the vacuum aperture is about 1.5:1, 2:1, 2.5:1, 3:1, or another ratio.

The vacuum aperture is optionally rounded in its contours, so as to avoid sharp interior corners. For example, it may be circular, oval, elliptical, or square or rectangular with rounded corners. For example, the smallest radius of curvature may be equal to or larger than about 0.25 mm, or about 0.5 mm. This provides a potential advantages for avoiding excess stresses on the tissue at the corners, which may tend to impede drawing tissue inward, and/or result in mechanical damage to the tissue.

In some embodiments, an electrode or other transducer that passes structurally disruptive energy (e.g., RF energy, acoustic energy, and/or heat energy directly) into tissue itself forms part or all of the vacuum aperture. For example, the electrode may form the vacuum aperture. It should be understood, particularly in relation to embodiments described as using vacuum to mechanically deform tissue, that an electrode used as a transducer for introducing structurally disruptive energy into tissue may optionally be replaced with another transducer type, e.g., a transducer which converts electrical power from an electrical energy source into the structurally disruptive form of the energy.

In some embodiments, the vacuum aperture and the electrode (or other energy transducer) are separately defined elements; for example, the electrode may apply RF energy through electrical contacts made external to the vacuum aperture and/or any chamber located behind it. In some embodiments, mechanical shrinkage of tissue prior to fixation by energy application relies not only on the ingress of tissue vacuum aperture, but also on a further mechanical motion of the vacuum aperture.

In some embodiments, for example, the vacuum aperture is advanced to adhere by suction contact with the tissue, and then withdrawn, pulling the tissue into a still further deformed shape. The deformation may be restricted by a bolster, for example, a protrusion that remains in contact more distally with tissue while the vacuum aperture is withdrawn. In some embodiments, the protrusion is a tube into which the vacuum aperture withdraws. In some embodiments, the tube is slit, potentially resulting in a fold or wrinkle which is confined across one axis by the enclosing slit, while across the other axis it can extend beyond the tube/slit boundaries. This potentially allows imposing a particularly large shrinkage ratio per lesion formed along one direction (e.g., the circumference), while the shrinkage ratio in the other direction remains relatively low. For example, the maximum shrinkage ratio in one direction may be up to 2:1, 3:1, 4:1 or higher. The maximum shrinkage ratio in the perpendicular direction may be lower than this by at least 25%, 50%, 75%, or another fraction.

A potential advantage of vacuum-formed mechanical distortion is that the electrode (or other energy transducer) operates from a superficial position. This may allow a somewhat more even thickness of ablated tissue to be achieved (at least, ignoring the vacuum-drawn “bump”) than when pin electrodes are used. A continuous healthy sheet of collagenous tissue may remain below the lesion, for example. Additionally or alternatively, that is potentially a lowered tendency to introduce focal flaws such as shape distortions or weak spots.

Optionally or alternatively, however, pin manipulation is used to mechanically deform tissue before providing energy to plastically set it into a new shape though one or more superficially acting electrodes (or other energy transducer) other than the pins themselves. It should be understood, particularly in relation to embodiments described as providing an exposed-surface contacting electrode (that is, an electrode separate from pins or other elements used to mechanically deform tissue), that an electrode used as a transducer for introducing structurally disruptive energy into tissue may optionally be replaced with another transducer type, e.g., a transducer which converts electrical power from an electrical energy source into the structurally disruptive form of the energy, for example as acoustic energy, light energy, heat energy, or another form of energy.

In some embodiments, pins may be provided which pinch together, slide linearly past each other, are rotatable, or otherwise can be moved to compress and/or otherwise deform tissue. Additionally or alternatively, tissue is secured for deformation by one or more releasable hooks, e.g., hooks with reversibly expanding barbs, and/or which pass into tissue along a path which curves at least partially back in a proximal direction. With two or more hooks curved in opposing directions, a minimum angle of curvature of each is optionally lessened, e. g., because tissue resists deforming, when the hooks are pulled on, to a degree sufficient to slip over the ends of the hooks. In some embodiments, straight hooks (which may be straight pins) are driven into tissue along non-parallel directions (e.g., converging or diverging directions). For example, tissue is hooked by trapping a wider region of tissue superficial to a deeper and narrower convergence of pins. However hooked and/or trapped, the penetrated tissue is pulled mechanically into a deformed position in contact with electrodes configured to deliver the structurally disruptive energy. For example, the hooks pull tissue into a recess, an inner surface of which at least partially comprises electrode surface area. Structurally disruptive energy is delivered through a surface of the tissue which is brought into contact with surface area of the superficially acting electrode(s), while pins and/or hooks are acting to mechanically deform the tissue.

### Guidance Features and Methods

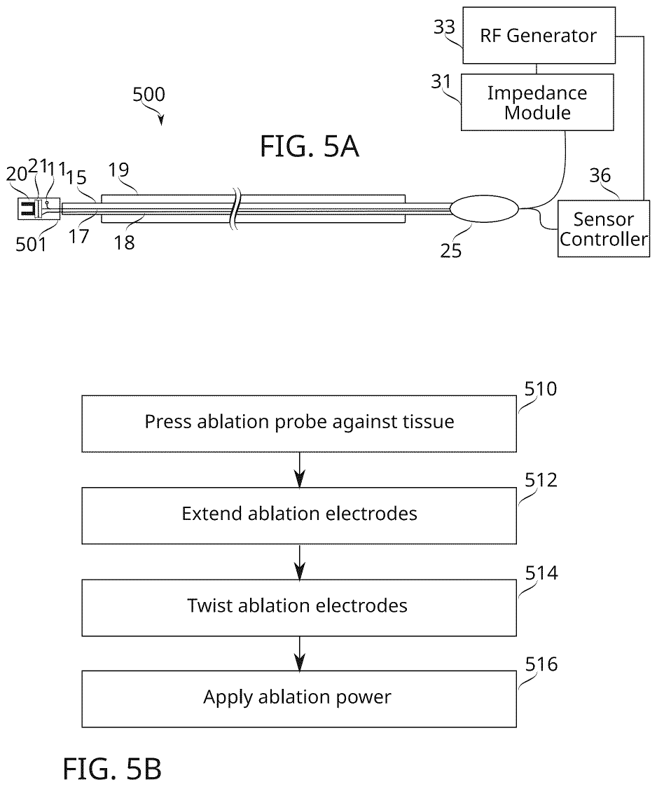

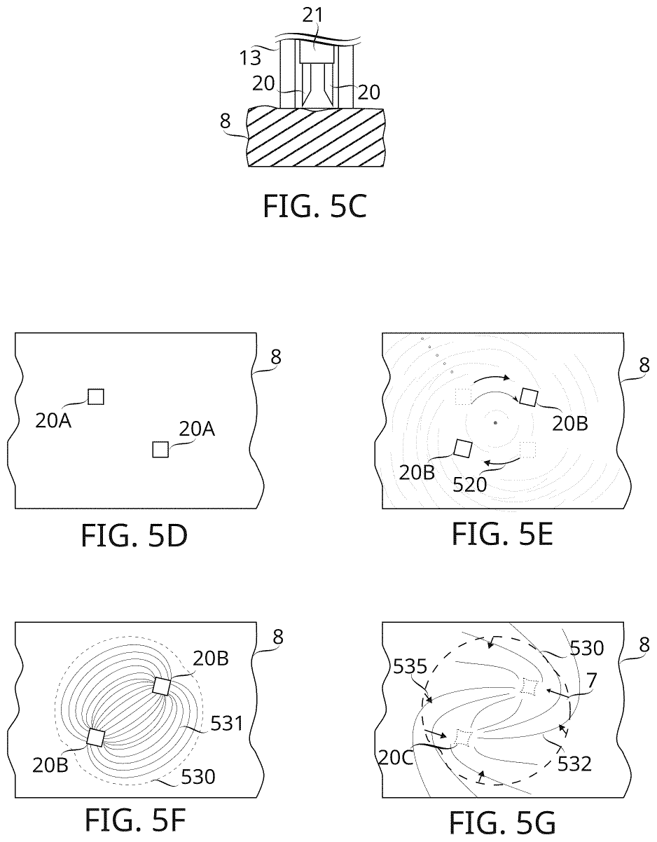

An aspect of some embodiments of the present disclosure relates to positioning and operating guidance features of a valve annuloplasty device, and/or methods of using these features, according to some embodiments of the present disclosure. In some embodiments, the valve annuloplasty device operates according to principles of tissue shrinking by a combination of twist/torsion (e.g., as described in relation to FIGS. 5A-11 herein) and the application of structurally disruptive energy. A full annuloplasty procedure generally comprises performing mechanical distortion and energy application at a plurality of sites around the valve annulus, referred to as treatment positions.

It is a potential advantage for a valve annuloplasty device operated over a minimally invasive catheter to include features which assist an operator in achieving and/or maintaining a robust sense of the present state of the annuloplasty device itself and/or the position relationship of the annuloplasty device with the valve annulus being treated. Related tasks include, for example, one or more of the following:
Bringing the annuloplasty device to a selected position around the valve annulus to begin a treatment, including ensuring the device is correctly positioned, e.g., in a correct initial orientation and/or offset relative the valve annulus.
Moving the annuloplasty device among a plurality of treatment positions which are to be mechanically distorted and subjected to structurally disruptive energy.
At each such position, suitably arranging the annuloplasty device so that it is ready to mechanically engage with tissue of the valve annulus beginning from a predictable insertion angle and/or to a predictable extent.
Actually mechanically engaging the tissue by the device, optionally including verification that the device is suitably engaged to perform mechanical distortion.
Performing the mechanical distortion, e.g., by twisting it and/or applying vacuum, optionally including verification that the mechanical distortion is occurring as intended.
Applying structurally disruptive energy to the tissue, optionally including verification that the energy is being received by the tissue as intended, and/or that the tissue is being disrupted as intended.
Withdrawing the device from the tissue, and proceeding to the next of the plurality of treatment positions.

It is a potential advantage to minimize complexity and/or uncertainty for each of these tasks. For example, complexity of tasks may be minimized by reducing the number of degrees of freedom (e.g., of device motion and/or device state) which are manipulated during a task. Other aspects in which task complexity may be simplified include reducing a range over which available degree(s) of freedom require adjustment, sequencing the manipulation of degrees of freedom (e.g., to be one at a time), and/or “quantizing” one or more of the available degrees of freedom.

In this context, the term “quantizing” should be understood to indicate that the implementation and/or sensing of a device degree of freedom allows the operator to select at least one definite predefined state; for example: needles fully withdrawn, needles fully advanced, needles non-rotated, and/or needles fully rotated. Partial states may also be quantized through use of sensing and/or mechanical methods such as detents. For example there may be half-rotated and/or half-advanced states of the needles defined.

The quantizing may be implemented by imposing physical constraints on the degree of freedom itself, for example by mechanical interference from a stop and/or detent (the detent defining a zone, optionally transient, of increased but not necessarily total resistance to further movement). In such a case, the operator may be freed of the need to attend to fine judgements of matters such as, e.g., if there is enough twist applied, or enough longitudinal advance. Instead, reaching the stop/detent (e.g., feeling increased resistance to further twisting/advancing) provides a potentially sufficient indicator. Additionally or alternatively, sensing and/or indications (e.g., electronic sensing and/or indication) may be used to detect and/or indicate the state of a degree of freedom of the device to the operator.

In some embodiments, an annuloplasty device is configured so that it tends to follow a path around the valve annulus as it is advanced by operation of some degree of control freedom. For example, a distal portion of the device may curve in a predetermined fashion as it is advanced from the constraints of an overtube. The curvature may be selected so that a tip of the device points successively at different regions around the annulus circumferences as the degree of control freedom is actuated. In some embodiments, there may also be a preconfigured degree of to which the device tip bends toward the plane of the valve annulus, e.g., starting from a position out of that plane such as an insertion point to a left atrium from a fossa ovalis of a heart intra-atrial septum. By such a configuration, the problem of positioning the device with respect to the three spatial dimensions may be significantly simplified largely to one of advancing or withdrawing. However (e.g., to allow fine adjustments), there may be provided one or more additional controls operable to modify the path (position) of the device tip.

Sensing itself need not be quantized. However, it is a potential advantage to have at least one indication which is of the “yes/no” type; e.g., a light that is on or off to assist in on the fly decision making. Additionally or alternatively, graded indications of sensed state are provided, e.g., a light that gradually changes in intensity and/or color as the sensed property changes. Sensed properties may include distance and/or angle of device actuation; implemented, for example, using electrical contacts and/or sensing of a resistance that varies with motion. Additionally or alternatively, a sensor may be configured to measure local electrical impedance, which has potential uses in confirming needle insertion into tissue, and/or tissue state as structurally disruptive energy is supplied to the tissue.

In some embodiments, an annuloplasty device comprises one or more elements which assist in its visualization though an imaging modality such as X-ray and/or ultrasound. For example, the annuloplasty device may be provided with one or more radiopaque markers shaped and positioned to assist in determining the orientation of the annuloplasty device tip. This potentially helps to ensure that the annuloplasty device tip is inserted at a preferred angle. For example, the preferred angle may comprise two needles of the device defining between them an axis having a direction which is angled obliquely to the circumferential direction of the valve annulus. The oblique angle may be about half of the total amount of angular change which the needles undergo when distorting the tissue by twisting it. For example, the needles are first twisted by the amount of the oblique angle until they are oriented with an axis between them which is perpendicular to the circumferential direction of the valve annulus; and then twisted further by about the same angle.

In some embodiments, the device tip in situ (e.g., while positioned within the heart chamber adjoining the valve to be treated) is rotated until two radiopaque markers mounted on opposite sides of the device tip reach a maximum distance, or a minimum distance (depending on viewpoint and/or how the device is configured. This may be the operating orientation of the device; or it may be used to calibrate the orientation of the device from which said orientation an offset may be added to select a targeted operating orientation. In some embodiments, when the operating orientation is set, direction of an axis extending between needles of the device is at the aforementioned oblique angle to the circumferential direction of the valve annulus. Optionally, the operating orientation is changed for different treatment positions, e.g., to modulate the amount of shrinkage which is applied at each treatments position, and/or to modulate the overall shape of the valve annulus (e.g., to bend it more or less toward a particular direction through a sequence of treatment positions). Optionally, the position of the needles relative to the radiopaque markers can be visualized fluoroscopically as well. This may be used to verify their relative state of advance from or retraction into the device tip, and/or their rotational state.

In some embodiments, one or more portions of the device tip are provided with structures which modify (e.g., increase) its ultrasound detectability; for example, sound scattering structures such as raised and/or inset stripes and/or divots. Optionally, these structures are varied on different parts of the device, e.g., varied along its elongated longitudinal extent and/or varied around some portion of its circumference. The contrast structures potentially assist in distinguishing (via ultrasound) what the device orientation is (e.g., relative to the ultrasound sensor, and transitively relative to the valve annulus via knowledge of the ultrasound's position relative to the heart). They potentially help to distinguish, e.g., the device housing and/or needles from the rest of the device. Optionally, the device tip is configured to enhance observation by ultrasound (“echo”), based on fluid extrusion into surrounding blood. In some embodiments, the device tip is supplied with fluid (e.g., infusion fluid) from a proximal side via an inner lumen. Flow of droplets at the device's tip is distinguishable by ultrasound, allowing visualization of device tip position.

In some embodiments, bio-impedance is measured during a procedure. This can be used, for example, to assess device contact with the valve annulus tissue, needle penetration of the valve annulus tissue, and/or the state of tissue disruption induced by the application of structurally disruptive energy. Optionally, it can be used to confirm that the tissue being contacted, penetrated, and/or disrupted actually is valve annulus tissue (e.g., insofar as the bio-impedance of this tissue differs from other nearby tissue such as leaflet tissue and/or cardiac muscle).

### Angle of Approach

An aspect of some embodiments of the present disclosure relates to operating a valve annuloplasty device using needle penetration followed by mechanical deformation and structurally disruptive energy application so that the needles approach (and then penetrate) the valve annulus from a direction which is to a large extent oriented from the center of the annulus outward, and potentially to a lesser degree from a position above or below the circumference of the annulus.

As a frame of reference, the direction from the annulus site of needle penetration to a geometrical center of the valve annulus may be set as 0°(the “shallowest” angle). The orthogonal directions at 90°/-90°(the “steepest” angles) represent directions pointing “up” or “down” away from the valve circumference. The “up” and “down” directions may be orthogonal or nearly orthogonal to the average plane of the valve annulus (or more particularly, the average plane of the valve circumference). This average plane is optionally defined as having the shortest average distance to the valve annulus circumference (e.g., as measured from the middle of the valve annulus thickness, or another reference location). In the case of the mitral valve, for example, “up” generally points into the left atrium, and “down” into the left ventricle. Relative to this angular axis, the needles are oriented to penetrate the tissue within a range of ±55°, in some embodiments. For example, the needles penetrate the tissue within a range of 0°to 55°when approaching the mitral valve from within the left atrium, e.g., after insertion the left atrium from the fossa ovalis and the interatrial septum. Practically, there may be a lower limit on the angle of approach, e.g., a lower limit in the range of about 20°-30°. Optionally, another range of angles is used, e.g., one limited to shallower angles (e.g., within +50°, +45°, or +40°), or one which includes steeper angles, e.g., within the range of +60°. In some embodiments, the angle of approach varies somewhat depending on circumferential location of the treatment position, with the shallowest angle being no more than about 15°, 20°, 25°, or 30°. The steepness of the steepest angle optionally does not exceed 45° and/or 60°. A range of 30°-40° may be considered “typical”, e.g., in some embodiments, at least half of all treated regions are approached at an angle within this range.

A potential advantage to the use of shallower angles of approach is that the device may be less likely to accidentally slip off the circumference of the valve annulus as it is pressed thereto. Moreover, a shallower direction of approach potentially helps ensure that penetration is into a relatively thick portion of the target. This may result in a lowered risk of injury to other tissue, and/or a greater volume of valve annulus tissue recruited into the changes induced by the annuloplasty treatment. There may also be a potential advantage insofar as the shallower direction of approach (e.g., from the atrial side of the mitral valve) may be more likely to glide over the valve leaflets than to end up unintentionally pressed against them. This potentially reduces changes for injuring the valve leaflets.

In some embodiments, e.g., as also noted elsewhere herein, the needles may moreover engage the valve annulus such that an axis extending between them is oriented obliquely to the valve circumference, e.g., oblique by about 45° in a direction extending above and below the valve circumference. A rotation by 90° in this case results in a final orientation also oblique by about 45°, preferably after passing through a position with the axis oriented perpendicular to the valve circumference in a direction extending above and below the circumference. This is not generally an orientation straight up and down, since the tilt of the axis in the radial direction is according to the shallowness of the angle of approach as described hereinabove. Other explanations herein relate to these same considerations using different descriptive approaches. These explanations should be understood as relating to mutually consistent conditions, at least insofar as one of them may clarify on how another of them is to be understood, in a case of substantive doubt about some aspect of the latter. Moreover, the descriptions may be combined (e.g., with respect to specific angular ranges mentioned and/or entities defined).

A larger or smaller angle of obliquity to the circumference than 45° (i.e., preparatory to more or less than 90° of total rotation) is optionally selected. Optionally, the arc of the rotation is not equal on either side of the circumference-perpendicular axis, that is, the midpoint is not necessarily perpendicular to the valve circumference. An “asymmetrical” rotation in this sense may be selected to potentially adjust how the annuloplasty procedure affects the planar bending of the valve, e.g., toward a more planar or less planar configuration.

Similarly, the clockwise or counterclockwise direction of twist is not necessarily selected identically at all treatment positions. Optionally, for example, opposite directions are chosen particularly for pairs of treatment positions flanking sites of (intended) valve leaflet coapting. For example, the roots of the valve leaflets below the treatment position are urged toward each other, albeit the tissue on the opposite side may be somewhat stretched. Even here, insofar as some tension potentially remain on the “stretched” side even after plastic remodeling due to the application of structurally disruptive energy, and/or develop during later recovery, there is a potential advantage for further increasing mechanical stabilization of coapting, specifically at the site marking the boundary between valve leaflets. Nor is it ruled out that effects associated with the (at least initially) “stretched” side are optionally induced, in some embodiments, on the side of the roots of the valve leaflet. For example, this may reduce bulging distortions, and/or assist in smoothing out the root region near where coapting occurs (or almost occurs). Potentially, controlling the pattern of clockwise vs. counterclockwise twisting promotes spreading and/or contracting leaflet shape, and/or a degree of undulation (i.e., of the valve circumference in and out of the average valve plane) around the valve circumference. Greater undulation may be selected to potentially increase effective circumferential shortening, albeit any such distortion generally should be controlled so that overall function (e.g., coapting) is not impaired.

In some embodiments, treatment positions are sited to be relatively biased toward the outer edges or inner portions of the annulus circumference of one or more of the leaflets (that is, towards or away from their coapting edges). This potentially helps to tune the effects of the annuloplasty on shapes that are adopted by the leaflet during the heartbeat cycle (and, coordinately, coapting). In this regard, it may be noted that the specific positioning of the pins relative to the circumference mid-point and/or shallowness of the angle of approach to the valve annulus may adjust the amount to which the root of the leaflet is recruited into the twisted region, and, accordingly, also potentially have an effect on coapting. This discussion of potential effects of treatment site selection, torsion arc (amount and/or orientation of twisting), and/or twist direction on coapting and/or other treatment effects should not be considered as commitment to a particular theory, and should be considered to be combined with (that is, build on, for some embodiments of the present disclosure) other discussions of annuloplasty effects presented herein, without necessarily limiting or being limited by the teaching of those discussions.

Before explaining at least one embodiment of the present disclosure in detail, it is to be understood that the present disclosure is not necessarily limited in its application to the details of construction and the arrangement of the components and/or methods set forth in the following description and/or illustrated in the drawings. Features described in the current disclosure, including features of the invention, are capable of other embodiments or of being practiced or carried out in various ways.

### Vacuum Attached Annuloplasty Devices

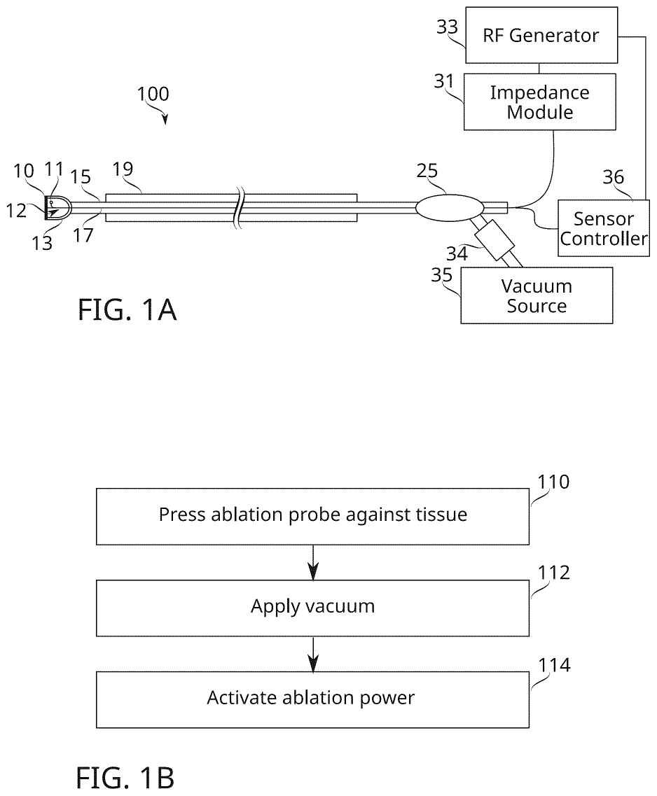

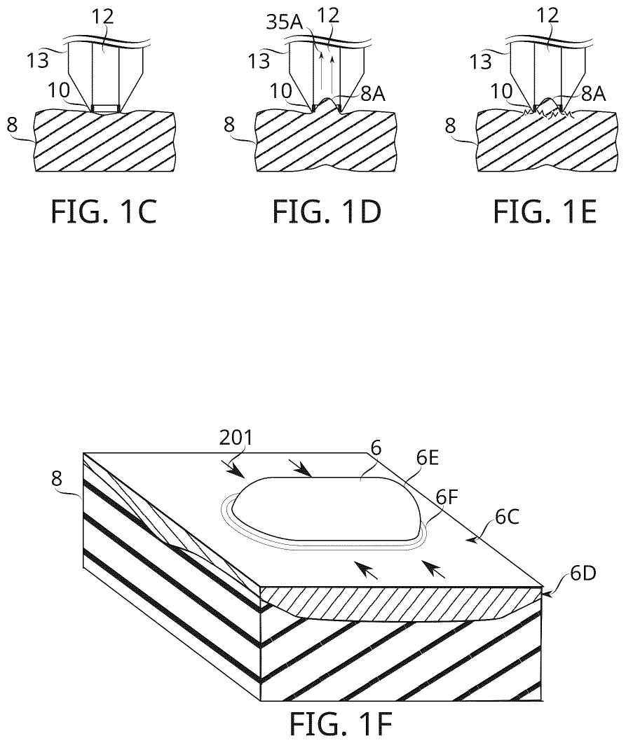

Reference is now made to FIG. 1A, which is a schematic illustration of a heart valve annuloplasty device 100 operable to shape valve ring tissue using vacuum, according to some embodiments of the present disclosure. In some embodiments, annuloplasty device 100 comprise a tip 13 comprising an electrode 10, and a chamber 12. Chamber 12 is open on a distal side. Chamber 12 is, furthermore, selectably in pressure communication with vacuum source 35 via a hollow of catheter 15. Selection is performed, for example, by operation of (optional) valve 34, by operation of a valve of vacuum source 35 (not shown) and/or by activation of vacuum source 35 itself. In some embodiments, valve 34 is a part of handle 25. In some embodiments, handle 25 includes appropriate passthrough conduits and valves to allow routing electrical power and vacuum separately.

Electrode 10 is also provided at a distal end of tip 13. It is in electrical communication with (i.e., receives electrical power for induction of fibrosis from) radio frequency (RF) generator 33 via electrically conductive wire 17 and optional impedance-matching module 31.

In some embodiments, steering of catheter 15 (i.e., steering to position tip 13 in apposition to a targeted region of a valve annulus prior to induction of fibrosis) is achieved by a steerable guiding sheath 19.

Alternatively or additionally, catheter 15 itself comprises a steering mechanism. Optionally, guiding sheath 19 is omitted in such embodiments. Handle 25 optionally includes controls to steer steerable guiding sheath 19 and/or catheter 15 Optionally, one or more sensors 11 are positioned at the tip, which may be connected back, e.g., via additional wires 17 to optional sensor controller 36. In some embodiments, the sensors 11 comprise a pressure sensor, which may be used to detect positive engagement with tissue. Optionally, control (e.g., interlock control) of RF generator is applied based on the sensing of pressure generated at tip 13 which is consistent with positive vacuum engagement with tissue. A pressure and/or flow volume sensor may be positioned in another location in pressure communication with the compartment under vacuum; e.g., along catheter 15, in handle 25, with valve 34, and/or as a component of vacuum source 35.

In some embodiments, sensors 11 comprise a temperature sensor, the output of which may also be used to assist in governing the operation of RF generator 33 to activate electrode 10. In some embodiments, sensors 11 comprise an impedance sensing electrode (and/or electrode 10 itself is used as an impedance sensing electrode), and the sensed impedance is used to assist in governing the operation of RF generator 33. For example, when sensed impedance is not at or near the level expected for collagenous valve annulus tissue, RF power generation may be locked out, and/or the device may enter a warning state. In some embodiments, expected impedance is distinguished, e.g., from the impedance of blood, and/or from the impedance of the leaflets. It is also noted, furthermore, that applied vacuum may draw the relatively thin and freely moving leaflets into tip 13 to a larger extent than the relative thick and fixed valve annulus tissue. In some embodiments, this is detected using one or more sensors 11 within chamber 12; for example, by placing the sensor at a position which the leaflet may reach, but not the valve annulus tissue.

Reference is now made to FIG. 1B, which is a schematic flowchart of a method of performing valve annuloplasty with the device 100 of FIG. 1A, according to some embodiments of the present disclosure. Further reference is made to Figures IC-IE, which schematically illustrate operation of the device 100 of FIG. 1A, according to some embodiments of the present disclosure.

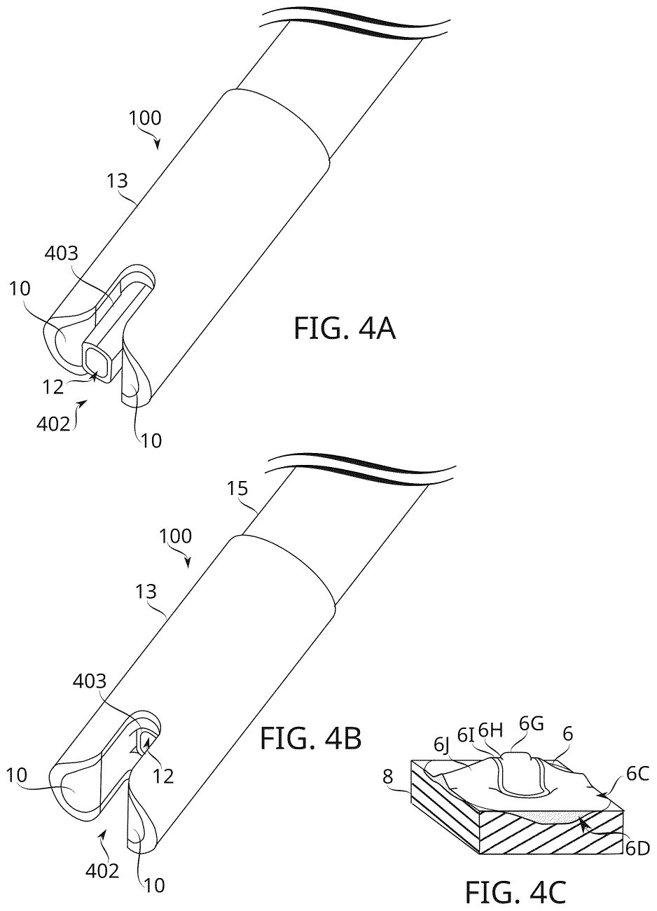

At block 110, in some embodiments, a tip 13 of annuloplasty device 100 is pressed against tissue (e.g., heart valve annulus tissue 200) targeted for induction of fibrosis and shrinkage. This corresponds, for example, to the situation shown in FIG. 1C, which illustrates (in cross-section) tip 13 placed against tissue 8. In the example shown, electrode 10 is located within chamber 12. In some embodiments, another relative positioning of electrode 10 and chamber 12 is provided, for example as described in relation to FIGS. 4A-4B.

The pressure need not be great, or even constant (e.g., in view of movements of the heart), so long as it is at some moment sufficient to permit the operations of block 112, as next described. Positioning of tip 13 may also comprise adjustment of its orientation relative to the circumferential direction and/or the axial height axis of the valve annulus. Where electrode 10 (and/or the aperture therethrough) is relatively elongated in one direction, shrinkage produced in a treated area may be greater (and/or more or less even) in one direction than another. This has potential importance for achieving a predictable result. It also has potential uses for tuning a procedure result. Orientation adjustment helps control this difference. The adjustment, in some embodiments, is controlled by rotating the whole handle of the device. In some embodiments, adjustment comprises rotating a knob or other control element. It is noted that the same fixed orientation of the device may be suitable for treating one region of a valve annulus, but (depending on how steering is performed) not necessarily a region, e.g., 90° away from it along the valve annulus circumference. Providing a specific control to manage rotation may help an operator keep track of the state of the device. In some embodiments, the preferred steering method is selected to maintain the same orientation of the tip no matter which side of the valve it is navigated to. For example, it may be preferred to navigate the catheter to the center of the valve, introduce a bend with a predetermined relationship to the orientation of the shape at the distal end of the tip, and then select different areas of the circumference by rotating the whole catheter while the steering angle remains bent. Rotating the catheter (e.g., by twisting its handle and/or an operating control thereon) by a set amount may then predictably adjust the orientation to allow its use, e.g., as a way of controlling how much shrinkage is generated along the circumferential direction of the valve annulus.

At block 112, in some embodiments, vacuum is applied to chamber 12, e.g., from vacuum source 35 via a lumen of catheter 15. This results in drawing tissue of the heart valve annulus 200 apposite to the opening of chamber 12, for example as illustrated in Figure ID. In Figure ID, vacuum force in chamber 12 is represented by arrows 35A, with the result that a greater pressure existing outside chamber 12 forces a tissue portion 8A of tissue 8 into chamber 12.

It may be noted that the resulting bulge of tissue portion 8A has a larger surface area than the surface area previously under chamber 12 (e.g., as shown in FIG. 1C). This is not entirely due to deformation of tissue underneath chamber 12; rather, deformation is induced both there and in adjacent regions of tissue 8. There is, accordingly, at least a small shrinkage of the surface adjacent to tissue 8, which, in some embodiments, comprises a portion of the annuloplasty-induced shape changes which are being performed.

At block 114, in some embodiments, RF power is delivered through the annuloplasty device. More particularly, in some embodiments, RF generator 33 is activated; and/or a switch connecting RF generator 33 to electrode 10 via wire 17 is closed. In some embodiments, impedance module 31 is provided to match the impedance of RF generator 33 to the impedance of the electrical sub-circuit comprising wire 17 and electrode 10.

The RF energy is provided at a frequency which interacts with tissue portion 8A and/or surrounding portions of tissue 8 to heat it. Energy is applied sufficient to denature the tissue, with the effect of generating plastic deformation and/or shrinkage, which remain in place after vacuum is released and tip 13 removed.

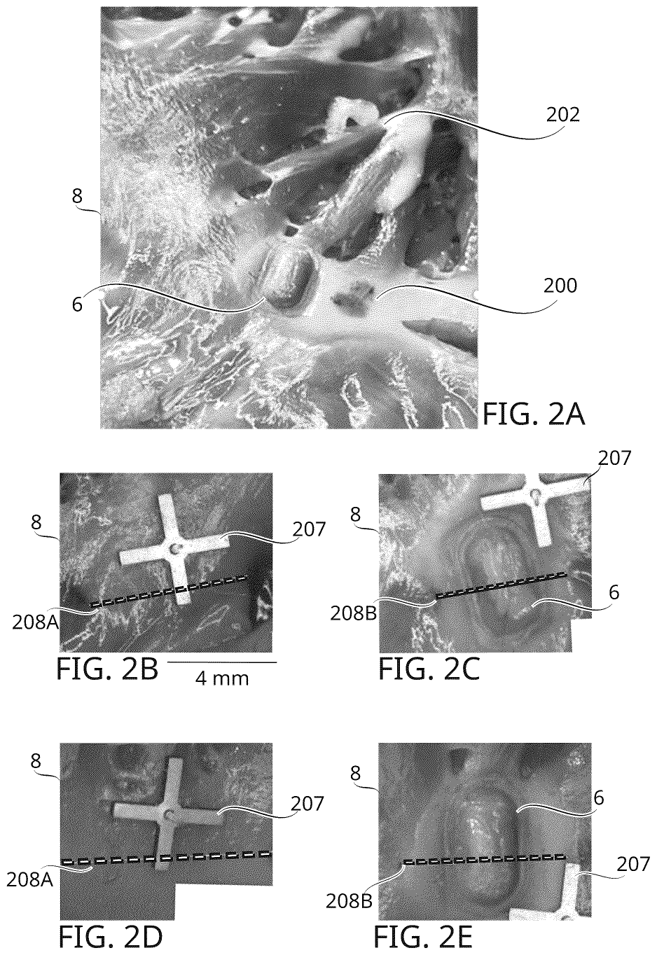

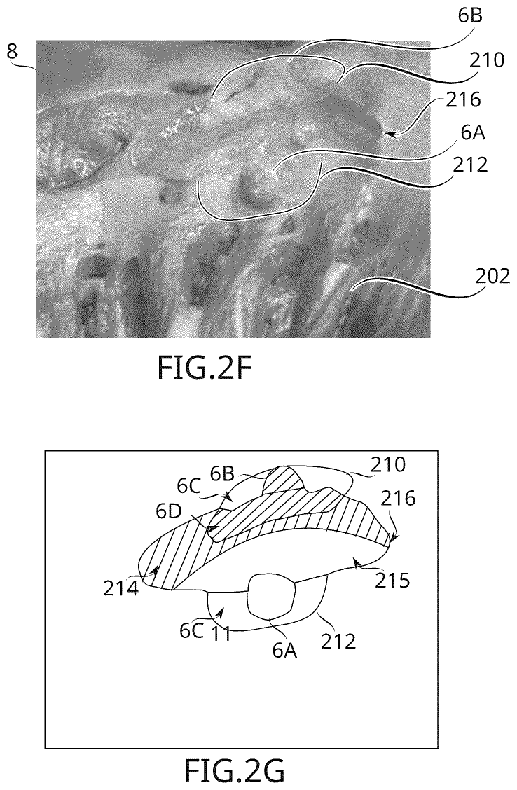

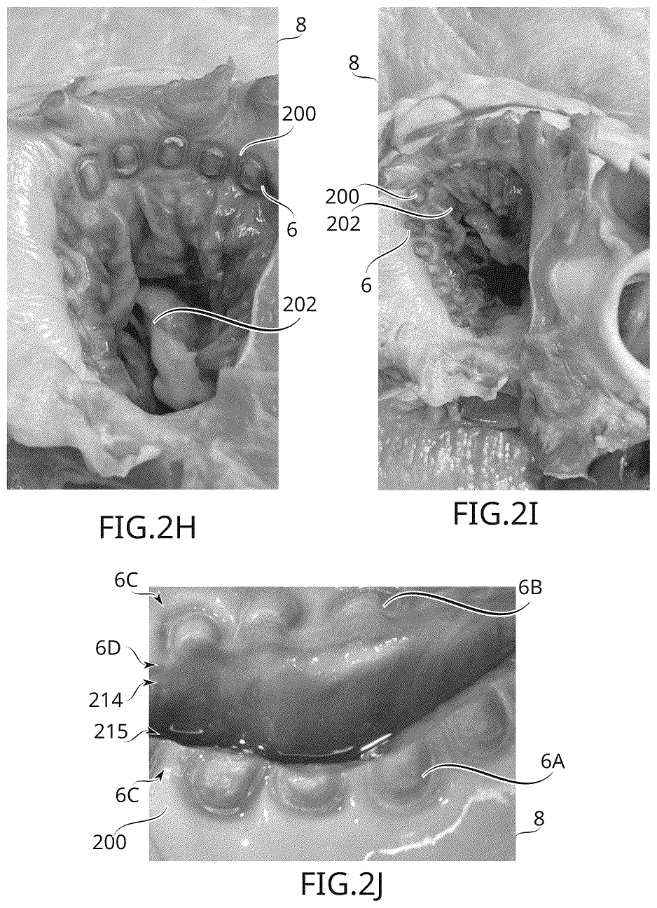

During an overall annuloplasty procedure, the operations of FIG. 1B (e.g., as illustrated by FIG. 1C-1E) are repeated multiple times, e.g., repeated to form a line or row of lesions 6 along an extent of the valve annulus 200 which is being subjected to remodeling of its shape and/or pliability. The row may extend partly around the valve annulus 200, or wholly around. Optionally, the row is divided into a plurality of row sections, marked by a relatively large inter-lesion gap between them. Examples of lesion rows are described, for example, in relation to FIGS. 2H-2J.

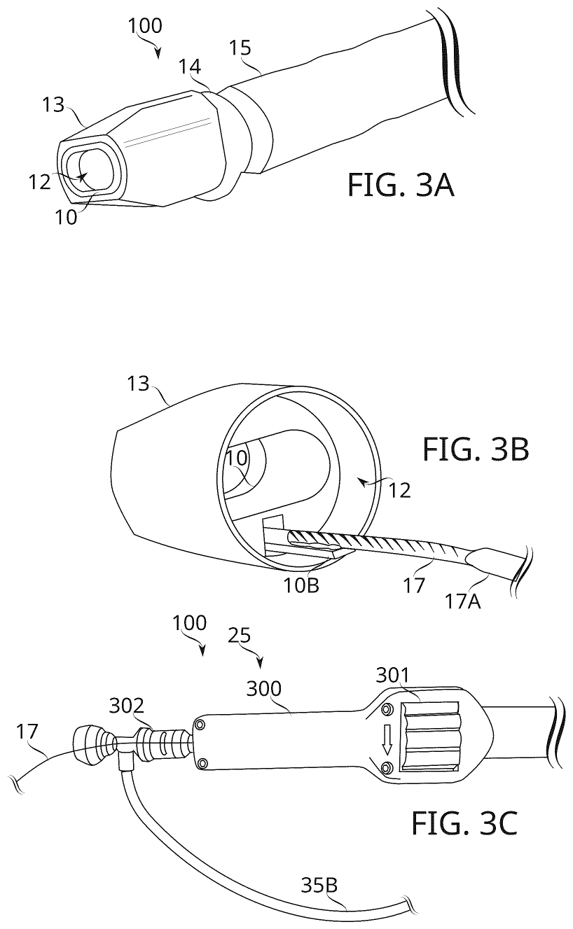

Brief reference is now made to FIG. 1F, which is a schematic illustration of a lesion 6 produced by the device 100 of FIG. 1A, according to some embodiments of the present disclosure. The vacuum-induced bulge of tissue portion 8A shown in FIGS. 1D-1E remains behind in the form of the upward bulge 6E that occupies the center of lesion 6. A region 6F “outlining” bulges 6E may also be produced, corresponding to locations where the distal surface of tip 13 contacted tissue 8. Furthermore, lesion 6 typically extends outward along surface 6C beyond the region of electrode contact shown in FIGS. 1D-1E, e.g., with a depth profile corresponding to region 6D. The lozenge-shaped bulge of lesion 6 corresponds, in some embodiments, to the shape of a lesion formed by an annuloplasty device 100 such as is described in relation to FIGS. 3A-3C. Although shrinkage may occur in and/or toward the center of lesion 6 from all directions, there is potentially a higher relative shrinkage by movement of tissue in the directions of arrows 201, toward either long side of bulge 6E. Potential reasons for this are described, for example, in relation to FIGS. 3A-3C.

Reference is now made to FIG. 2A, which is a photograph of a lesion 6 produced ex vivo in porcine heart tricuspid valve using device 100, according to some embodiments of the present disclosure. More particularly, the lesion was produced using a heart valve annuloplasty device 100 constructed with a tip 13 as described in relation to FIG. 3A-3C. Lesion 6 was produced in tissue 8 according to the method of FIG. 1B, and more particularly, produced within a region of valve annulus tissue 200 which is a portion of a heart valve 202. Shapes corresponding to bulge 6E and surrounding outline 6F are clearly seen.

Reference is now made to FIGS. 2B-2E, which are before-lesioning (FIGS. 2B, 2D) and after-lesioning (FIGS. 2C, 2E) photographs of regions of lesions 6 produced ex vivo in porcine heart tricuspid valve using device 100, according to some embodiments of the present disclosure. The 4 mm scale bar shown in association with FIG. 2B applies also to FIGS. 2C-2E. Fiducial mark 207 in each of these figures is itself 4 mm across. In each of FIGS. 2B, 2D, dotted line 208A represents a surface width of tissue before lesioning shown in FIGS. 2C, 2E, respectively. In each of FIGS. 2C, 2E, dotted line 208B shows the corresponding post-lesioning width of the region marked by the corresponding line 208A. Lesion 6 occupies the middle of the shrunken region, with features visible that correspond to bulge 6E (about 2.3 mm wide and about 4.3 mm high) and to outline 6F as shown in Figure IF. The extent of lesioning corresponding to the surface extent 6C of lesion 6 may be identified extent of the slightly discolored region of lowered glossiness around the central bulge.

In examples performed like those shown in FIG. 2A-2F, the relative shrinkage of regions corresponding to lines 208A was determined to be in the range of 20.5% to 26.5%, corresponding to an absolute nominal shrinkage of 1.2-2.1 mm (depending also on the overall region size affected). Ablation power used was 8 Watts, for a time of 18 seconds.

Reference is now made to FIG. 2F, which is a photograph of a sectioned lesion 6 produced ex vivo in porcine heart tricuspid valve using device 100, according to some embodiments of the present disclosure. Further reference is now made to FIG. 2G, which schematically outlines regions corresponding to features of the photograph of FIG. 2F, according to some embodiments of the present disclosure. For clarity, only outline lines 210, 212 are also shown in FIG. 2F. These may be used to help orient to identify features labeled in FIG. 2G.

Tricuspid valve elements shown in the photographs correspond more generally to a heart valve 202.

Lesion portion 6B comprises about half of a bulge of a lesion 6 on one side of a slit 216 made through the lesion 6 roughly along a line corresponding to lines 208B if FIGS. 2C and 2E (the upper side, as shown). Lesion portion 6A corresponds to the other half of the lesion bulge. Looking down into slit 216, the relatively dark and/or glossy slit wall 214 corresponds to non-lesioned regions of tissue. The lighter and/or less glossy region 6D illustrates the depth profile of lesioning through the exposed cross-section, having a width of about 7.2 mm, and a depth from surface 2C of about 1.5 mm. The bulge of lesion portion 6B rises about 1.5 mm above surface 2C. Outline lines 212, 212 roughly delineate (on either half of the sectioned lesion) the superficial extent of lesioning corresponding to the surface 6C of the overall lesion 6 indicated in FIG. 1F. Region 215 comprises the exposed floor of slit 216.

Reference is now made to FIGS. 2H-21, which are photographs of rows of lesions 6 produced ex vivo in porcine heart mitral valve using device 100, according to some embodiments of the present disclosure. Reference is also made to FIG. 2J, which is a photograph of a sectioned row of lesions 6 produced ex vivo in porcine heart mitral valve using device 100, according to some embodiments of the present disclosure.

Heart mitral valve elements shown in the photographs correspond more generally to a heart valve 202, e.g., valve annulus 200 is an annulus of a mitral valve. The lesions 6 correspond to lesions made with power of from 8-12 Watts, over a period of 12-18 seconds. Generally lower times are associated with correspondingly higher wattage, so that the overall energy used is approximately constant, between about 144 Joules and 162 Joules.

In the examples shown, the long axis of each lesion 6 is oriented along the axis of blood flow through the valve. Optionally, the lesions are placed alongside each other with a spacing between the raised bulges of about 2 mm (e.g., about 85% of the width of an individual bulge). Optionally a larger or smaller spacing is used.

The depth of lesioning may again be seen in depth of the relatively light region 6D, extending laterally (left to right) above darker region 215, which is itself shown above slit floor 215 in FIG. 2J.

However, there is no particular requirement that the lesions produced be contiguous.

In this example, the lesioned area 6C is substantially continuous. Placing lesions 6 at least close enough for the affected tissue areas to touch helps ensure that a maximum amount of shrinkage available through a particular circumferential section of the valve annulus 200 is produced. However, this is optional, e.g., based on whether or not using the full extent of available annulus remodeling is needed to ensure coaptation. It is noted in particular that annuloplasty may be targeted to specifically reduce valve annulus circumference in a region bridging two valve leaflets which coapt poorly, while reduction of valve annulus circumference in another area may be ineffective at reducing regurgitation.

Diminishing returns are expected for lesions placed close enough to produce “double lesioning” (a plurality of exposures to heating at a level that induces fibrosis) in regions shared between them. The returns diminish at least insofar as lesioning shrinkage is a function of changes comprising shrinkage of the tissue itself which are not additive upon repeated treatment. Nevertheless, the shape changing which vacuum induces (e.g., the surface area contraction represented by the bulge) may confer some additional valve circumference reduction as lesions become more closely spaced.

Reference is now made to FIG. 3A, which is a schematic illustration of a distal end of a heart valve annuloplasty device 100, according to some embodiments of the present disclosure. Reference is also made to FIG. 3B, which is a schematic illustration of a distal-ward view of a tip of a heart valve annuloplasty device 100, according to some embodiments of the present disclosure. Further reference is made to FIG. 3C, which is a schematic illustration of a handle and connection region of a heart valve annuloplasty device 100, according to some embodiments of the present disclosure.

A heart valve annuloplasty device of the design of FIGS. 3A-3C was used to create the lesions shown in and/or described with respect to FIGS. 2A-2J. In FIG. 3A, a distal end of catheter 15 is shown, together with adhesive 14 used to secure it to tip 13. Tip 13 is of a roughly frustoconical design, with an initially circular cross-section narrowing slightly in diameter from a proximal side to a distal side. Furthermore, toward the distal side, the cross-section flattens on two opposite sides, so that tip 13 terminates with a rectangular cross-section with fully rounded corners (that is, the rounded corner portions of the perimeter meet each other on the short sides of the rectangle). In some embodiments, tip 13 is made of PEEK, or another material (e.g., another polymer) which provides good electrical insulation combined with heat resistance. Increases in one or both of these material properties may allow tip 13 to be produced with relatively thinner walls, potentially reducing its overall size.

Inset into this distal cross-section of tip 13 is a similarly shaped (but smaller) electrode 10, which is open in its center to provide access to chamber 12. The inner aperture through electrode 10 is about 2×4 mm. In some embodiments, electrode 10 has a wall thickness of, e.g., about 0.5 mm, and a wall height (extending proximally) of about 1 mm.

The inner aperture may be slightly smaller than the (e.g., 2.3×4.3 mm) lesion bulge 6E that is actually produced, potentially due to a slight relaxation of lesioned tissue after the vacuum hold is released. The wall thickness of electrode 10, together with the surrounding wall thickness of tip 13, helps define the size of lesion-surrounding region 6F.

The inner aperture shape of electrode 10 of FIG. 3A has an oblong aspect ratio (aspect ratio >1, e.g., about 2). This aspect ratio greater than 1 potentially helps to encourage greater relative shortening of a valve annulus across the short dimension, compared to across the long dimension. For example, across the short dimension, if the intrusion of the tissue portion 8A under vacuum is about 2 mm through a circular cross-section, then the ratio of bulge circumference to bulge width is about 1.6. Along the long dimension, the ratio (assuming quarter-turn circle shapes at either end) is closer to 1.3. As a result less remodeling per unit length is needed lengthwise to produce the given shape. This can be helpful, e.g., when the long axis of the inner aperture of electrode 10 is oriented along the direction of blood flow through the valve, where the valve annulus is shorter compared to length around its circumference. For example, this may help to reduce distortion in that direction due to uneven shrinkage, where there is less overall distance to “even out” such differences. It is noted that the actual shrinkage ratio achieved overall is somewhat smaller than just described, e.g., due to post-treatment relaxation, and/or because some of the lesion is outside the bulge portion of the lesion.

The rounded corners of the inner aperture of electrode 10 provide a potential advantage for allowing tissue to be smoothly pulled into chamber 12 under suction. For example, cutting and/or compression of intruding tissue at the corners of the aperture may be reduced.

In the proximal-side view of tip 13 shown in FIG. 3B, a portion of wire 17 is shown as comprising a wound cable of strands; attached (e.g., crimped, soldered, and/or welded) to the rest of wire 17 proximally through connector 17A. On the distal side, wire 17 is attached (e.g., crimped, soldered and/or welded) to an extension 10B, which may be an extension integrally formed together with electrode 10 (e.g., cast together with and/or cut from the same stock). Optionally extension 10B is itself attached to electrode 10, but separately formed.

FIG. 3C shows a proximal portion of annuloplasty device 100, comprising handle 25. Handle body 300 is sized to be held in the hand. Knob 301 may be used (e.g., finger-adjusted) to adjust an adjustable valve which controls whether vacuum is presently applied to chamber 12. The vacuum is received, in some embodiments, from vacuum source 35 via tubing 35B. An isolation valve 302 attaches tubing 35B to handle body 300; e.g., to a proximal side of the handle, or another location which may be convenient to keep it out of the way during operation of handle 25. Isolation valve 302 also allows wire 17 to pass out of it, on the way to the connection of wire 17 with RF generator 33 (e.g., connection via impedance module 31). A handle with these (and optionally additional) features may be optionally provided to any embodiment of an annuloplasty device 100, e.g., the device of FIGS. 4A-4B.

Reference is now made to FIGS. 4A-4B, which schematically illustrate of a distal end of a heart valve annuloplasty device 100 with the vacuum attachment element 403 in advanced (FIG. 4A) and retracted (FIG. 4B) positions, according to some embodiments of the present disclosure.

In general, the annuloplasty device 100 of FIGS. 4A-4B includes the features described generically for annuloplasty device 100 in relation to FIG. 1A, and is used as generally described in relation to the method of FIG. 1B. However, compared to the embodiment of, e.g., FIGS. 3A-3C, electrode 10 and chamber 12 are moveable with respect to each other. In the example shown, chamber 12 is defined by a movable element 403 which translates longitudinally along a proximal-to-distal axis relative to electrode 10.

When advanced distally, vacuum applied to chamber 12 secures apposed tissue to it, as described with respect to block 110 of FIG. 1B). An additional operation may then be performed whereby element 403 is withdrawn proximally. Secured tissue is thereby pulled deeper into the tip 13, into a recess 402 which has a somewhat larger size than element 403. In effect, withdrawing element 403 potentially allows a deeper bulge to be pulled than for the example of FIGS. 3A-3C. Recess 402 optionally takes the form of a slit, so that tissue shape along one axis is relatively less constrained, even while tissue shape along the other axis is deformed more extensively.

Electrode 10, in some embodiments, extends from a distal region of contact with tissue into slot 402. Lesioning, accordingly, may be performed along contacts of electrode 10 with deformed tissue which are everywhere substantially outside chamber 12. This potentially result in the formation of lesion bulges which look like pinched-up pleats or folds in the tissue, e.g., rising relatively abruptly (as well as high) above the base surface when moving along the valve circumference, but more gradually when moving along the axis of blood flow, for example as shown in FIG. 4C.

Reference is now made to FIG. 4C, which schematically illustrates a lesion 6 produced by the device 500 of FIGS. 4A-4B, according to some embodiments of the present disclosure. The lesion 6 is potentially somewhat taller than shown in FIG. 1F. It may be surmounted by a nodule 6G formed by tissue withdrawn fully into chamber 12 itself, which is potentially still lesioned (and plastically deformed as a result), even though not in contact with the electrode 10 directly. The surface of region 6H (surrounded by surround 6I) is mostly in direct contact with electrode 10 when lesioned; pulled into contact with electrode 10 by traction from tissue in nodule 6G, although not itself subject to direct vacuum. Region 6I forms the remainder of the pleat. It may comprise a mixture of lesioned tissue (more centrally) and non-lesioned tissue (peripherally) which is passively drawn into a new shape due to traction from other collapsed areas. Region 6C indicates that the surface of the lesion may extend significantly outside what is directly in contact with electrode 10. The cross-sectional views of Region 6D on the front and side indicate lesion depth.

The “folded” shape of such a lesion shape has the potential advantage of allowing larger changes in valve annulus circumference overall, and/or of focusing changes in valve circumference to regions of particular importance, e.g., at and/or surrounding junctions between valve leaflets.

### Annuloplasty Devices Using Rotational Distortion

Reference is now made to FIG. 5A, which is a schematic illustration of a heart valve annuloplasty device 500 operable to shape valve annulus tissue using needle penetration and torsion, according to some embodiments of the present disclosure.

In some embodiments, annuloplasty device 500 comprise a tip 501 comprising pin electrodes 20, optionally recessed until extended from tip 501. Pin electrodes 20 are in electrical communication with (i.e., receive RF electrical power from) radio frequency (RF) generator 33 via electrically conductive wire 17 and optional impedance-matching module 31. In some embodiments, pins 20 are stabilized to allow them to exert torque on annulus tissue sufficient to deform it without excessively deforming themselves. For example, pins 20 are mounted to a mounting block 21. Mounting block 21 may hold the pins at a fixed distance from one another while allowing them to rotate around a common axis. In some embodiments, pins 20 are rotated under control exerted through control member 18 from, e.g., a control on handle 25. For example, control member 18 may pull on mounting block 21 to rotate it. Optionally, functions of control member 18 and wire 17 are combined. Actuation to rotate pins 20 may make use of mechanical advantage, for example, via a gear, lever, and/or pulley mechanism. The mechanism may be embedded in the tip 501 itself, may be embedded in the mechanisms of handle 25, and/or may be distributed; for example, there may be provided a pulley mechanism comprising a plurality of lengths of control member 18 passing up and down catheter 15 in order to gain mechanical advantage.

In some embodiments, handle 25 includes appropriate controls to operate pin electrodes mechanically, e.g., to extend or retract them, and/or to rotate them around a longitudinal (proximal-distal) axis of tip 501.

In some embodiments, steering of catheter 15 (i.e., steering to position tip 501 in apposition to a targeted region of a valve annulus prior to treatment) is achieved by a steerable guiding sheath 19. Alternatively or additionally, catheter 15 itself comprises a steering mechanism. Optionally, guiding sheath 19 is omitted in such embodiments. Handle 25 optionally includes controls to steer steerable guiding sheath 19 and/or catheter 15.

Optionally, one or more sensors 11 is be provided with tip 501, optionally connected with sensor controller 36, which may in turn operate to assist governing operation of RF generator 33 to deliver power to pin electrodes 20. Sensors 11 may, for example, sense temperature and/or impedance. Effects on the governing of operation of RF generator 33, may be, for example, as described in relation to FIG. 1A.

Reference is now made to FIG. 5B, which is a schematic flowchart of a method of performing valve annuloplasty with the device 500 of FIG. 5A, according to some embodiments of the present disclosure. Further reference is made to FIGS. 5C-5F, which schematically illustrate operation of the device 500 of FIG. 5A, according to some embodiments of the present disclosure.

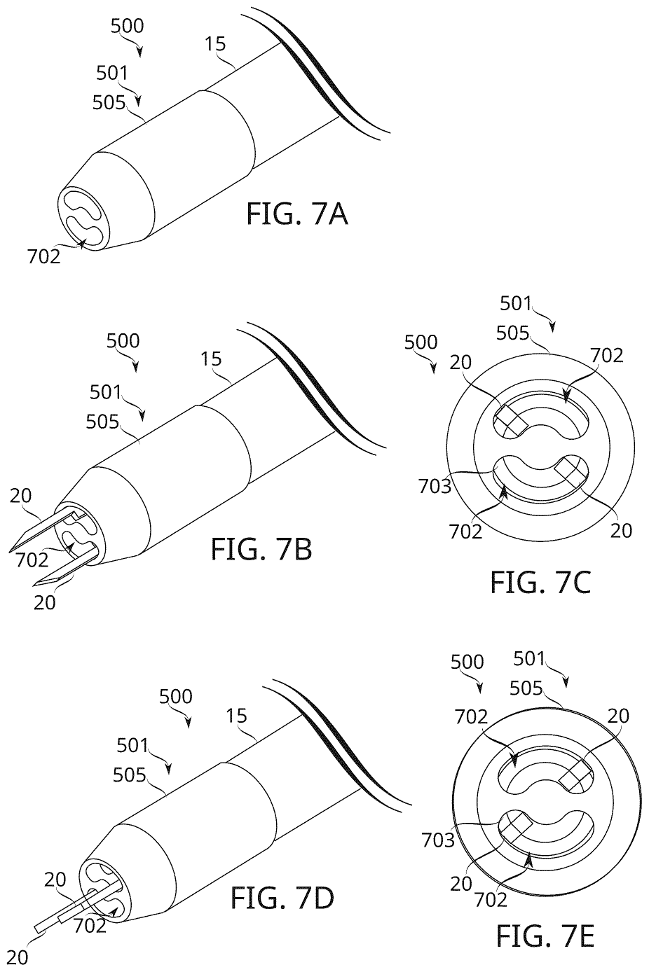

At block 510, in some embodiments, tip 501 of annuloplasty device 500 is pressed against tissue (e.g., heart valve annulus tissue 200) targeted for shrinkage. This corresponds, for example, to the situation shown in FIG. 5C, which illustrates (in cross-sectional view) tip 501 placed against tissue 8. In the example shown, electrodes 20 are recessed to within a housing of tip 501, e.g., mounted on a retractable mounting block 21. In some embodiments, mounting block 21 is also rotatable under control from handle 25. This and other tip configurations are described, for example, in relation to FIGS. 7A-7E, 8A-8B, and 10A-10C.

Optionally, at block 512, in some embodiments, pin electrodes 20 are extended into the tissue 8. This corresponds, in some embodiments, to the situation shown in FIG. 5D, wherein the pin electrodes 20 are inserted into the tissue at positions 20A (for this and the rest of FIGS. 5D-5G, illustration of the remainder of the annuloplasty device is suppressed for clarity of illustration). In some embodiments (e.g., as described in relation to FIG. 11), the inserted elements manipulate tissue mechanically, but do not themselves pass RF energy, for example as described in relation to pins 1102 of FIG. 11. In some embodiments and/or uses, pin electrodes 20 do not retract or are left extended, and block 512 is optional. However, it is a potential advantage for the pin electrodes to be retractable during navigation and/or positioning, e.g., the positioning of block 510.

Particularly once pin electrodes 20 are inserted to tissue, It is not necessary that any housing of tip 501 remain in direct contact with the target region; e.g., it may be withdrawn slightly while at least a portion of pin electrodes 20 remain in position. this can be used, e.g., to adjust lesion depth.

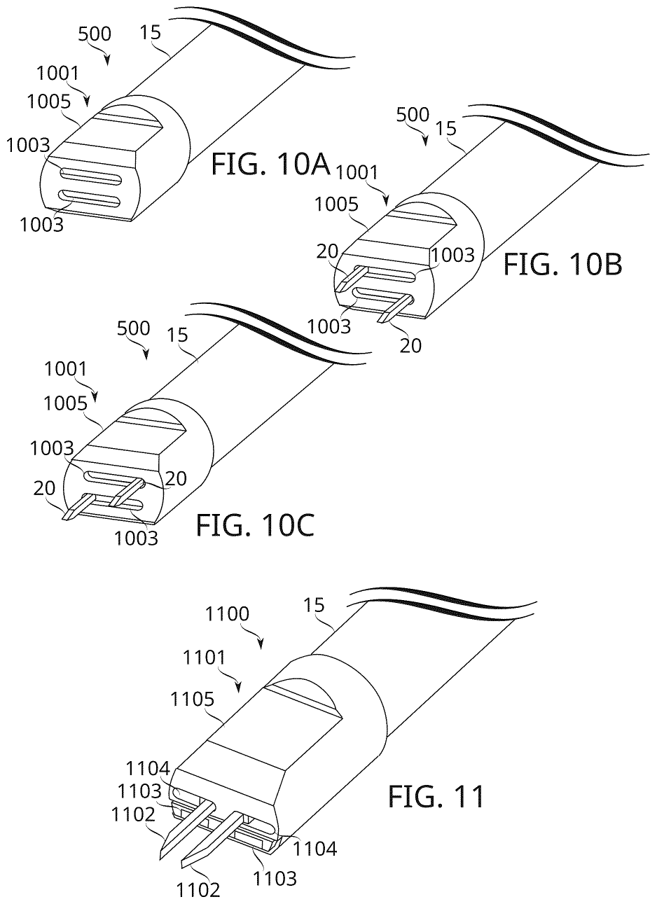

At block 514, in some embodiments, torsion (twisting) is exerted on tissue 8 through pin electrodes 20, e.g., to rotate some portion of tissue 8 by up to about 60°-120° around a proximal-distal axis of tip 501. Pin electrodes 20 themselves rotate, in some embodiments; for example, they may rotate about a common center. Alternatively, in some embodiments, pin electrodes 20 do not rotate, but instead translate (e.g., translate toward each other). They translate in such a way, however, that torsion is induced in the tissue, for example as described in relation to FIGS. 10A-10C.

The operations of block 514 correspond, for example, to the illustration of FIG. 5E, in which the pin electrodes 20 move from positions 20A to positions 20B, generally by moving around a circular path indicate by arrows 520. This produces twist and “swirling” in the tissue 8, which tends to compress it, e.g., as discussed in relation to FIG. 5G. The flat-sided (e.g., square) cross-sectional geometry of the pin electrodes potentially assists in gripping tissue to impart twist, however a circular or other cross-section is optionally used in some embodiments of the present disclosure.

At block 516, in some embodiments, RF power is provided. The RF power may comprise RF energy exerted through the pin electrodes. This corresponds, in some embodiments, to the situation illustrated in FIG. 5F, with field lines 531 representing RF transmission in the region where it is concentrated enough to induce lesioning. Dotted line 530 represents the lesion extent. Effects of lesioning include converting the transient deformation due to twisting into a more permanent plastic deformation (and net shrinkage). Lesioning may also itself induce volumetric shrinkage of tissue.

Lesioning energy used may be, for example, in the range of about 100-200 Joules, for example, 140-160 Joules. Examples of combinations of power and time within this range include, for example, 8 Watts for 18 seconds, 10 Watts for 15 seconds, and 12 Watts for 12 seconds. Using relatively higher power may help concentrate lesioning effects (e.g., avoiding energy loss due to thermal conduction). Conversely, using relatively lower power may allow increasing the area of effect without generating charring.

Brief reference is now made to FIG. 5G, which is a schematic illustration of a lesion 7 produced by the device 500 of FIG. 5A, according to some embodiments of the present disclosure. Bounds of the lesion are demarcated by dotted line 530. Puncture holes 20C indicate damage left behind by the pin electrodes 20. In the figure, they are positioned to match pin electrode position 20B, but in actual cases there may be some spring-back as non-lesioned tissue seeks to return to its equilibrium state.

Arrows 535 indicate directions of compression due to twisting. In large part, but not exclusively, compression may be manifested in a wrinkled tissue surface. Shrinkage may be increased also by volumetric effects of lesioning on tissue structure. Arrows 535 are drawn transversely to the local direction of wrinkles 532, which are indicated in FIG. 5G to provide an example of how tissue may deform as a result of twisting.

Some tissue “piles up” into wrinkles 532 ahead of the rotating pin electrodes 20 as advancing tissue nearby each pin electrode 20 is compressed against the relatively immobile tissue beyond it. In this location, the longitudinal orientations of peaks and valleys of wrinkles 532 tend to be near the tangent to circles centered on the nearby pin electrode 20. Tissue drawn behind a pin electrode 20, although under tension along an axis in the direction of the pin electrode 20, also compresses along the (roughly) orthogonal axis, since it is drawn partially inward as it follows. In these locations, wrinkles 532 tend to be oriented nearer to the radial direction. It is noted that tension mechanisms play a large role in shaping the deformation pattern produced by twisting (torsion); even a portion of the compression in the pattern is potentially attributable to tissue under tension in one direction “crowding together” (e.g., wrinkling) in the orthogonal direction as tension re-aligns it. While compression mechanisms tend to be vulnerable to unstable buckling even within the limits of material integrity (e.g., a small bend can give rise to greater structural weakness which in turn makes the bend larger), tension mechanisms lack this failure mode. This potentially enhances reproducibility of torsion pattern results, and may help avoid, e.g., “jumping” (and potential disruption of control and/or interference with its “feel”) which might occur when compressed tissue suddenly buckles into a new configuration.

The deformations set up in the tissue may favor twist shrinkage (e.g., as manifested in tissue wrinkling) along a particular axis (anisotropy). This may be especially true locally, and emphasized if the electrode pins are rotated by much less than a full turn (e.g., about 90° or less). For example, in some embodiments, the fan of “drawn behind” wrinkles tends to distribute tension forces over a large area, which may include regions outside the eventual lesion. The “pushed ahead” wrinkles may tend to concentrate tissue distortions more locally. In some embodiments, accordingly (e.g., for an approximately 90° twist), the axis of greatest shrinkage is roughly parallel to an axis drawn between the starting and ending positions of each pin electrode. In the case of FIG. 5G, an axis extending horizontally may experience more shrinkage than an axis extending vertically, for example.

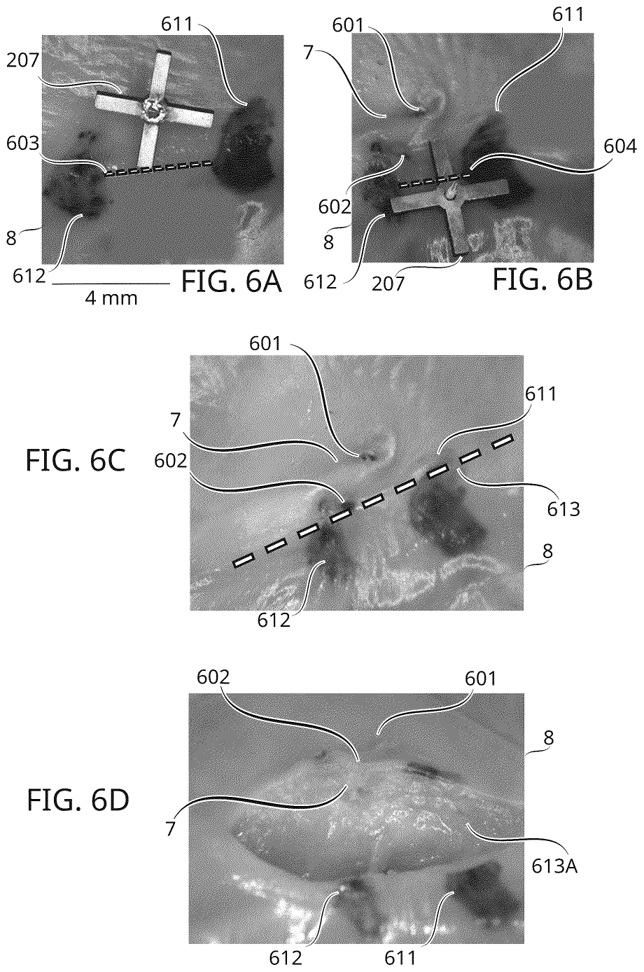

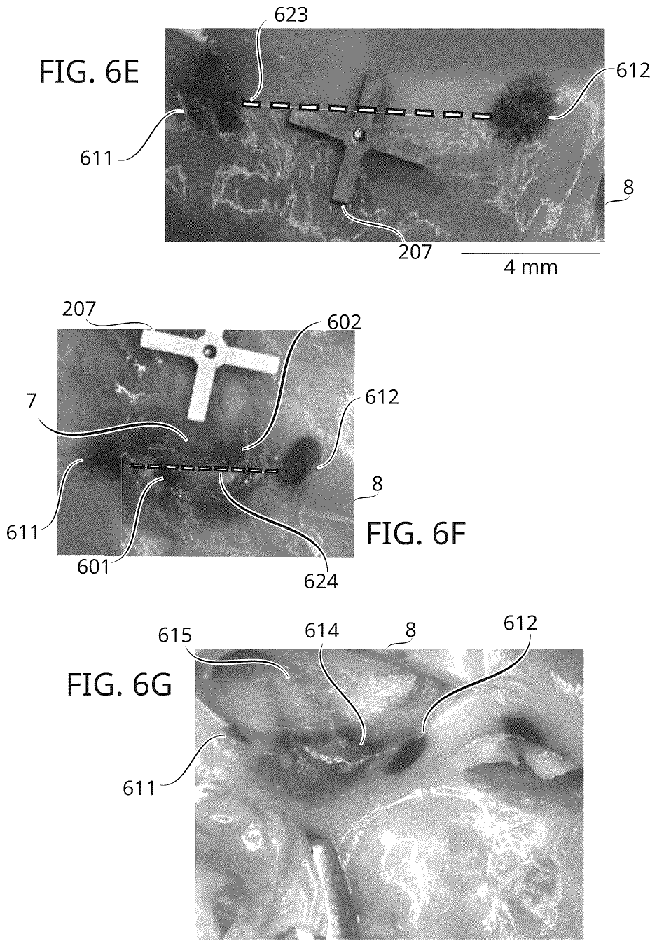

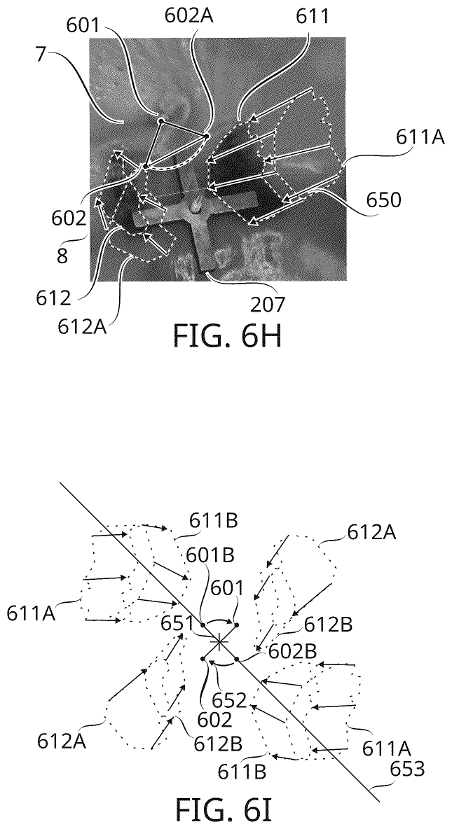

Reference is now made to FIGS. 6A-6B, which are before-lesioning (FIG. 6A) and after-lesioning (FIG. 6B) photographs of the region of a lesion 7 produced ex vivo in porcine heart using device 500 of FIG. 5A, according to some embodiments of the present disclosure.

In the example, two dye-stain marks 612, 611 were made in the tissue 8 before lesion formation. Line 603 (FIG. 6A) represents their distance before lesioning, and line 604 (FIG. 6B) represents their distance afterward. The difference in sizes was determined to be about 41%, or about 1.5 mm. In both relative and absolute magnitudes, this compares very favorably, for example, to shrinkage induced using the vacuum-pulling method, while resulting in a potentially lower profile of the lesioned area (less raised-up relative to the surrounding tissue). Lower profile lesions produce a potential advantage, for example, by reducing concerns about interference with hemodynamic flow through the valve.

In FIG. 6B, lesion 7 is centered on punctures 601, 602; made about 2.0 mm apart from each other. Ablation used 8 Watts for 18 seconds. Fiducial mark 207 indicates the scale size of 4 mm (e.g., as the scale bar in associate with FIG. 6A is drawn).

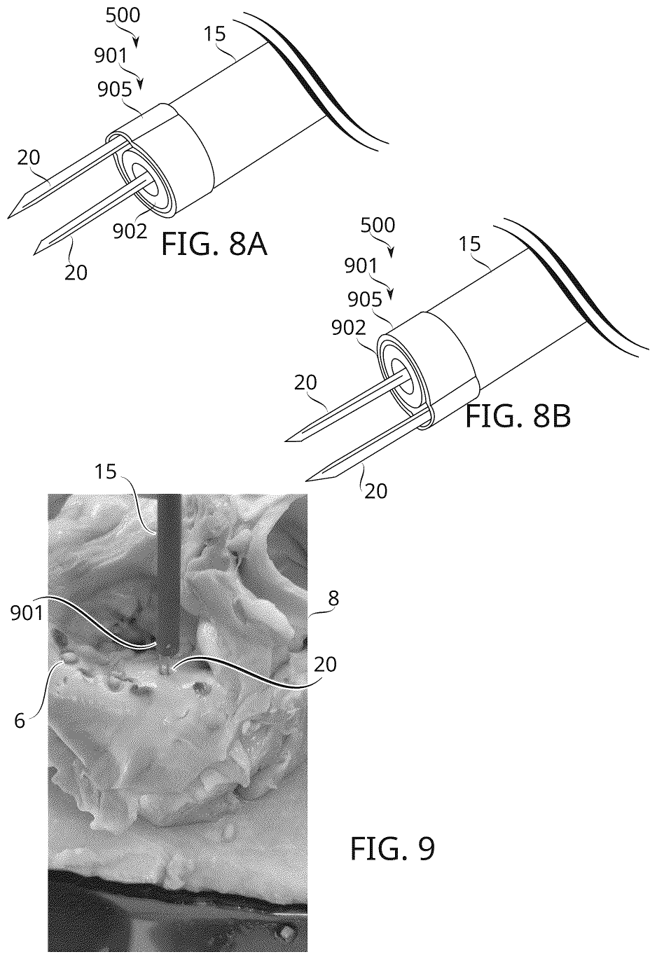

In the example of FIG. 6B, one of the electrode pins 20 that shaped the tissue before induction of fibrosis was stationary, and the other traveled circumferentially around it; for example, as described in relation to the embodiments of FIGS. 8A-8B. This type of motion may assist in the predictability of the resulting pattern of shrinkage for a user, who will, in actual use, often be relying on indirect visualization methods such as X-ray and/or ultrasound images that provide indistinct and/or partial information about what is occurring. For example, one pin electrode can be visualized as if moving between its starting point and its ending point in a substantially straight line (even though it may actually curve), while the other pin can be visualized as fixed. This may make it more apparent to the device operator ahead of time how tissue will be dragged behind the moving pin electrode along a distance approximately equal to the starting/ending point distances, while tissue ahead of it is compressed.

Nevertheless, the resulting pattern of tissue distortion may be quite similar to and potentially indistinguishable from elements of the wrinkling pattern described in relation to the symmetrical twist described in relation to FIG. 5G. For example, there is a fan of relatively small wrinkles (alternating light and dark bands) extending off the top of the image. Nearer to electrode puncture 601, a large wrinkle with the characteristics of “pushed ahead” compression appears to its right and below, extending left and between punctures 601 and 602 to connect up with a compression wrinkle associated with puncture 602. Patterns of twisted tissue movement which may occur in some embodiments of the present disclosure are also described, for example, in relation to FIG. 6H-6I.

Brief reference is made to FIGS. 6C-6D, which are photographs of the before-(FIG. 6C) and after-(FIG. 6D) sectioning appearance of the lesion 7 of FIG. 6B, according to some embodiments of the present disclosure.

FIG. 6C generally reproduces the situation of FIG. 6B, with fiducial mark 207 removed from the frame. Cut line 613 represents the sectioning line which produced the cut 613A shown opened in FIG. 6D. The depth of lesion 6 may be judged from the depth extent of the less glossy portion of tissue extending downward, e.g., from at about the position of puncture 602.

Reference is now made to FIGS. 6E-6F, which are before-lesioning (FIG. 6E) and after-lesioning (FIG. 6F) photographs of the region of a lesion 7 produced ex vivo in porcine heart using device 500 of FIG. 5A, according to some embodiments of the present disclosure. Reference is also made to FIG. 6G, which is a photograph after sectioning of the lesion 7 of FIG. 6F, according to some embodiments of the present disclosure.

Line 623 (FIG. 6E) demarcates the pre-treatment distance between dye-stain marks 612, 611; scaled by 4 mm-wide fiducial mark 217, a distance also indicated by the 4 mm scale bar associated with FIG. 6E. In FIG. 6F, line 624 indicates the decrease in the same distance after treatment according to the method of FIG. 5B, from a position indicated by punctures 601 and 602, positioned about 1.8 mm apart from each other. The average shrinkage for 6 such tests was about 2.5 mm, or 46% across the width of the lesion. Ablation power used was 8-10 Watts, for a time of 18-20 seconds.

The slit-open region 615 dividing dye-stain marks 611, 612 in FIG. 6G indicates the depth of the lesion made in tissue 8, in this case as the darkened region 614 visible at the center lower margin of the aperture of slit-open region 615. The fibrotic region itself is slightly lighter in color than a much darker surrounding area which was only partially affected by the treatment.

Reference is now made to FIG. 6H, which represents estimated motions of tissue and pin electrode positions superimposed on the lesion image of FIG. 6B, according to some embodiments of the present disclosure.

The same features as are shown in FIG. 6B are indicated (except that line 604 is suppressed), together with an overlay. The overlay indicates the original pin electrode position 602A which moved during rotation through the indicated linking arc (dotted lines) to puncture position 602, Also shown are dotted outlines indicating the original positions 611A, 612A of the dye-stain marks 611, 612; the final positions of the dye-stain marks are also outlined.

Arrows 650 link several before-and-after positions. The frame of reference used for rotation is the position of puncture 601. For purposes of assigning the relative placement of before and after positions, the two images were aligned as if all rotation occurred around this point. Selecting another center of rotation as the frame of reference (for example as shown in FIG. 6I) is compatible with the same pin electrode positions, and the same before-and-after states, but attributions of movements are distributed differently.

The choice of frame of reference in FIG. 6H is not the only one available (e.g., as described in relation to FIG. 6I), but may assist understanding of what can happen to the structure of tissue as rotation compresses it. As pin position 602A swings clockwise to puncture 602, it drags with it tissue on the right hand side, for example, as indicated by the movement of outline 611A into the outline of dye-stain mark 611. The arrows 650 linking them are about the same length as the distance between 602A, 602. They could be shorter if the tissue were more elastic. They could be longer, e.g., if there were enough “disk like” (rigid) circumferential movement at radii larger than the radius of the pin electrode movement. Relatively little of that appears in this example.

In contrast, tissue from the positions of outline 612 moved mostly up and leftward in this frame of reference. This can be understood in part as tissue being compressed leftward by the movements of the pin electrode(s), insofar as tissue further to the left is well enough supported to resist being moved itself. The tissue is also rotating somewhat here (accounting for the upward motion), creating a shrinkage component partially in the orthogonal direction to the movements from 611A to 611, insofar as tissue further toward the top (e.g., above dye-stain mark 612) relatively anchored (in this frame of reference) by the presence of a pin at puncture 601.

Reference is now made to FIG. 61, which represents estimated motions of tissue and pin electrode positions using a different frame of reference for the movements of FIG. 6B, according to some embodiments of the present disclosure.

In this case, the rotational center 651 chosen for use in before and after image alignment is the +-marked midpoint of the segment joining punctures 601, 602. Punctures 601, 602 are shown in their final positions, after about a 90° rotation from positions 601B, 602B in the direction indicated by the curved arrows 652.

Outlines 611A and 612A are the same as for FIG. 6H, duplicated by 180° rotation for purposes of explanation and visualization. Outlines 611B, 612B are the outlines of the dye-stain marks 611, 612 according to their relative positions in, e.g., FIG. 6B. Arrows 650 link before and after positions of various points of the dye-stain mark outlines, which indicate different particular directions and distances of movement than in FIG. 6H, which is due to the frame of reference chosen; the starting and ending states are the same.

In this frame of reference, the motions are symmetrized, so that the two pin electrodes are both perceived as moving around their common midpoint, in the same clockwise direction. Since the pin movements are symmetrical, the original positions of the outlines 612A, 611A, 612B, 611B (at bottom) can be treated as approximately repeated in similar fashion above. In reality, there are likely to have been deviations from symmetry due, e.g., to inhomogeneous distribution of forces that the tissue 8 exerts upon itself.

The pattern shown emphasizes in particular how tissue moved in relation to axis 653, which intersects the original pin electrode positions 601B, 602B. In the lower right quadrant, tissue move mostly toward the left (and by symmetry, toward the right in the upper left quadrant. There was some (but relatively little) radial movement inward. In contrast, on the lower left, motion was upward and to the right (slightly more upward), with radial movement dominating circumferential deflection. Reverse motions are repeated at the upper right to complete the view. Considering the net effects resulting, there is apparent inward movement toward rotational center 651 in all quadrants, but in the lower right/upper left quadrants, the movement is almost completely horizontal, while in the lower left/upper right quadrants, the movement is about equally horizontal and vertical (with vertical slightly dominating). This suggests that tissue compression was strongest in the horizontal direction, and weaker vertically.

The resulting shrinkage anisotropy, in some embodiments, is controlled so that maximum shrinkage occurs along the circumference of a valve annulus; for example, axis 653 is set so that rotation from positions 601B, 602B to positions 601, 602 is symmetrical about the vertical axis. In the case shown, that is about a 45° angle off the axis of the vertical axis, for a movement through about 90°.

Reference is now made to FIGS. 7A, 7B, and 7D which schematically illustrate a distal end of a heart valve annuloplasty device 500 in different operating states, according to some embodiments of the present disclosure. Further reference is made to FIGS. 7C and 7E which schematically illustrate end-on views of the operating states of FIGS. 7B and 7D respectively, according to some embodiments of the present disclosure.

In this example embodiment of an annuloplasty device 500, tip 501 comprises a housing 505 attached to catheter 15 on a proximal side, and terminating distally with a frustoconical taper. The distal surface of tip 501 comprises two slot apertures 702. Slot apertures 702 are each elongated along a portion of the outer circumference of the distal surface of tip 501, and wide enough to accommodate pins electrodes 20, as shown in FIG. 7B.

In FIG. 7A, pin electrodes 20 are retracted, which may facilitate navigation of the device to the site of treatment, optionally up to and including placement of the device against the targeted treatment site (i.e., corresponding to block 510 of FIG. 5B.

In FIG. 7B, pin electrodes 20 are extended (e.g., corresponding to the operations of block 512 of FIG. 5B), each through a separate respective slot aperture 702, and positioned near the counter-clockwise edge of its slot aperture 702 as seen from the distal side (left for the top pin electrode 20, right for the bottom pin electrode 20). This corresponds also to the configuration shown in the view of FIG. 7C looking proximally along a proximal-distal axis of tip 501, in which there is also shown a support 703 within the housing 505 of tip 501, and to which each pin electrode 20 is attached.

Rotation of support 703 within housing 505 can be actuated (e.g., under control from handle 25), to move pin electrodes 20 each to the opposite (clockwise edge) side of its respective slot aperture 702, as shown in FIG. 7D, and its corresponding view in FIG. 7E looking proximally along a proximal-distal axis of tip 501.

During the operations of block 514 of FIG. 5B, support 703 and its supported pin electrodes 20 are rotated between the configurations of FIGS. 7B-7C and FIGS. 7D-7E (either clockwise or counterclockwise). This imparts a twist to tissue, for example as described with respect to the lesions of FIGS. 6B and 6F. Slot apertures 702 restrict the extent of rotational motion to about 90°, which is a potential advantage for providing reproducible effects, for example, reliable in terms of geometrical effects (e.g., degree of shrinking), and/or reliability of performance (e.g., reducing a likelihood of tearing, slipping, and/or excessive deformation).

In some embodiments, a plurality of annuloplasty devices 500 are provided (e.g., as a kit), each having slot apertures 702 extending through a different full-range angle (e.g., to allow pin electrode 20 movements limited to from about 45° up to about 120°. Any suitable number of options may be provided, for example, between 4 and 8 options. This may assist in tuning a procedure to achieve a certain amount of valve annulus shrinkage per treatment site; e.g., a surgeon can calculate the product of treatment site number and shrinkage per treatment site in order to achieve a surgical plan which is both feasible and expected to produce the desired amount of remodeling. Additionally or alternatively, in some embodiments, slot apertures 702 are themselves adjustable, for example by the rotation of two slotted plates relative to each other. Perfect alignment of their slots may result in the largest available slot aperture, or the slots may be somewhat misaligned to limit the available range of motion. Optionally, slot alignment is set before insertion of the catheter to the target region. In some embodiments, slot alignment is performed by operation of a control remote from tip 501, e.g., available on handle 25.

In some embodiments, the slot apertures 702 are provided so as to always be large enough, and motion range is selected to smaller values in another way. For example, a control on a handle 25 may be provided which can be rotated to a particular position before insertion. After the pins are inserted into tissue at that angle, whatever amount of rotational range is left in the slot apertures 702 sets the range of motion available.

A slot aperture is not necessarily involved in setting rotational range, e.g., as described in relation to embodiments of the type described with respect to FIGS. 8A-8B. Motion may be entirely determined by operator selection at the moment of rotation, or motion may be limited by stops on a control element and/or a controlled element. There is a potential advantage to using a stop which limits the motions of pin electrodes 20 directly (by contacting them and/or their rigidly attached support 703), e.g., insofar as the distance it allows is insensitive to backlash which may occur through control linkages. In some embodiments, additional to and/or in place of a slot aperture, another stop element may be provided as part of tip 501; for example, one or more internal protrusions that interfere between housing 505 and support 703, one or more internal protrusions of housing 505 that interfere directly with the pin electrode 20, or another stop arrangement. Stop arrangements may also provide tactile feedback allowing the operator to feel when a limit is reached. In some embodiments, one or more intermediate détentes are provided which allow tactile sensing of partial rotational state, and/or provide confirmation that rotation is occurring.

Noting that pin electrodes 20 are electrically conductive, their motion may be verified via one or more electrical contacts made with a sensing conductor electrically interconnected with a proximal side of the device, thereby providing a sensing switch which can be directly or indirectly coupled to the activation of an audible and/or visual indicator such as an LED. In some embodiments, rotation is sensed with resolution that allows distinguishing one or more intermediately rotated states, for example, using Hall effect sensors, multiple encoding contacts, a potentiometer (rotary variable resistor), or another method.

It is noted that applying torsion to the whole handle 25 potentially results in the transmission of twisting along catheter 15; thus, in case there is a perceived need to exert additional torsion in a particular case, the option remains available at least in principle by bypassing the self-limited mode of operation.

It is also noted that the relative orientation of an axis extending through the two pins and the circumferential and/or axial height directions of the valve annulus is optionally adjusted in this fashion, e.g., as a part of the Reference is now made to FIGS. 8A-8B which schematically illustrate a distal end of a heart valve annuloplasty device 500 in different operating states, according to some embodiments of the present disclosure. Further reference is made to FIG. 9, which is a photograph of the heart valve annuloplasty device 500 of FIGS. 8A-8B in use during ex vivo lesioning of a porcine heart valve, according to some embodiments of the present disclosure.

In this example, tip 901 comprises a cylindrical housing 905 attached at a proximal side to catheter 15, and open at its distal side. Electrode pins 20 are shown already extended, but may be retractable; for example as described with respect to block 512 of FIG. 5B and/or the embodiment of FIGS. 7A-7E.

In some embodiments, rotation of housing 905 relative to support 902 (e.g., under control from a handle 25) changes the relative positions of the two pin electrodes 20, optionally to any arbitrary relative angle.

For example, a rotation of about 180°is shown in the difference between the configurations of FIGS. 8A and 8B; optionally a different rotation is performed, e.g., a rotation in the range of 60°-120°, e.g., about 90°. In some embodiments, the pin electrode 20 inside the lumen of support 902 is rotationally fixed; confined by the lumen of support 902 but not necessarily rotated by it around its own axis. The outer pin electrode 20 (occupying a space between support 902 and housing 905) may be attached to and move along with rotation of housing 905 (e.g., as shown), or it may be attached to support 902. In the latter case, case housing 905 may be configured to rotate relative to catheter 15 upon rotation of support 902, such that support 902 and housing 905 rotate together. Alternatively, housing 905 may be provided as a cylinder without the bulge shown accommodating outer pin electrode 20, allowing support 902 to rotate eccentrically within housing 905 to change the relative locations of the pin electrodes 20. Optionally, housing 905 is rotatable relative to catheter 15 while support 902 (and the pin 20 it supports) remains fixed.

It may be noted that in the embodiments of FIGS. 8A-8B, the center around which the pin electrodes 20 rotate is, effectively, also a center of one of the pin electrodes 20. For example, such an embodiment was used in forming the lesion of FIG. 6B. Accordingly, and in contrast with the example illustrated in FIGS. 5D-5G, one pin electrode 20 (e.g., occupying puncture 601) may stay in place, while the other pin electrode 20 (e.g., occupying lesion 602) goes around it, with the appearance of “winding” tissue around the stationary pin. Since this induces different tissue movements with respect to the frame of reference of the stationary tissue, there may be some difference in the appearance of the resulting wrinkle pattern. Taking puncture 601 as the center reference, for example, it appears that puncture 602 was rotated counterclockwise around it.

FIG. 9 shows a portions of an annuloplasty device 500 comprising a distal end of catheter 15 and tip 901, with pin electrodes 20 extended and partially inserted into tissue 8 in preparation for twisting and lesioning. Nearby may also be seen several lesions 6 with lozenge-shaped protrusions, produced separately by a different annuloplasty device 100.

Reference is now made to FIGS. 10A-10C which schematically illustrate a distal end of a heart valve annuloplasty device 500 in different operating states, according to some embodiments of the present disclosure.

In this example, tip 1001 of an annuloplasty device 500 comprises a housing 1005 attached at a proximal side to catheter 15, and having two straight slot apertures 1003 on its distal side through which electrode pins 20 may optionally be retracted (FIG. 10A) or extended (FIGS. 10B-10C). They remain extended if retraction is not implemented. The two electrode pins 20 are positioned on opposite sides of their respective slot apertures 1003.

Actuation of the device (e.g., from a handle 25) swaps the sides of the electrode pins 20. Although motion of the electrode pins 20 is linear, the effect on tissue is to impart twist. There is an intermediate period of linear compression combined with torsion as the electrode pins 20 cross past each other. Optionally, actuation is stopped at the intermediate position before structurally disruptive energy is applied.

### Annuloplasty Devices Using Surface Electrodes

Reference is now made to FIG. 11, which schematically illustrates a distal end of a heart valve annuloplasty device 1100, according to some embodiments of the present disclosure. This example has a tip 1101 which separates functions of electrical energy conduction and tissue arrangement. Pins 1102 move (e.g., while embedded in tissue as has been described for pin electrodes herein) in slot apertures 1104 of tip housing 1105. They close together from their initial separated position with the pins at the outer edges of the slot apertures 1104 to the compressed position shown in FIG. 11. Tissue to which they are inserted is likewise compressed, and the compressed shape is retained upon plastic deformation induced by the treatment.

The RF energy is, however, sent through tissue separately via electrodes 1103. These are held in contact with the tissue surface adjacent to the pins 1102 while operated. The separation has the potential advantage of avoiding placing the highest temperatures (which may weaken tissue) at the positions of highest mechanical stress. This potentially allows larger compression to be applied; for example, shrinkage of a 5 mm-wide region to 2.0-2.5 mm wide.

Another potential advantage of this approach is to decouple the design of the lesion shape from the design of the tissue-shaping device. Electrode pins, for example, generate lesions along their penetration depth, while surface electrodes can be operated to keep the lesion more superficial. This may also help deep tissue around the inserted pins to retain its mechanical integrity during treatment that induces fibrosis in a more superficial layer.

Since they do not need to bear mechanical stress, the surface electrodes can be narrow (e.g., less than half a mm wide), to potentially produce a correspondingly narrower lesion. Additionally or alternatively, the surface electrodes can be larger in the width direction than is suitable for a pin electrode (e.g., together occupying half or more of the width of the probe, which may be, for example, 4-7 mm wide overall). This potentially allows current to be distributed more evenly over the region to be treated. In turn, this may help to reduce damage due to excessive focal heating, and/or improve the homogeneity of the lesion generated. Potentially, the administered energy (e.g., power or operating time) can be shorter. This can be, for example, because of the more widespread contact patch that distributes the energy more evenly, and/or since there is less need to ensure that regions more than a short distance (e.g., a millimeter or so) from the probe are reached.

### Construction of an Annuloplasty Device Using Rotational Distortion

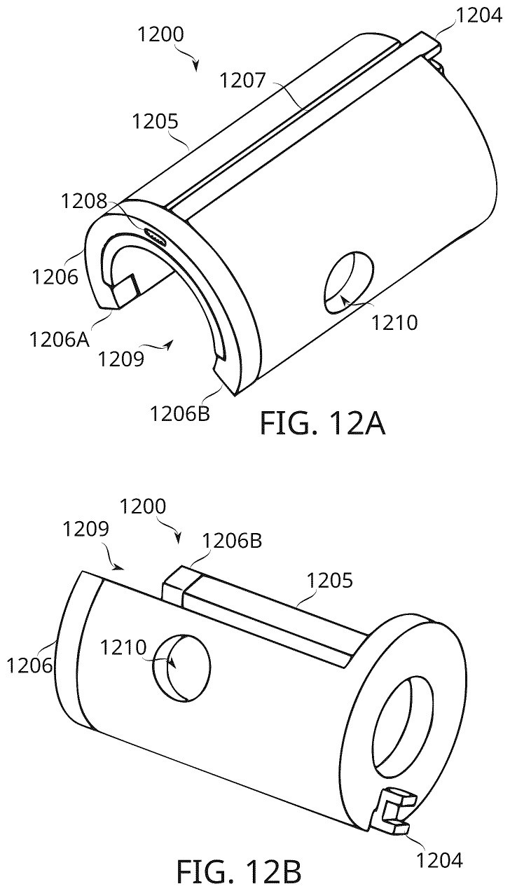

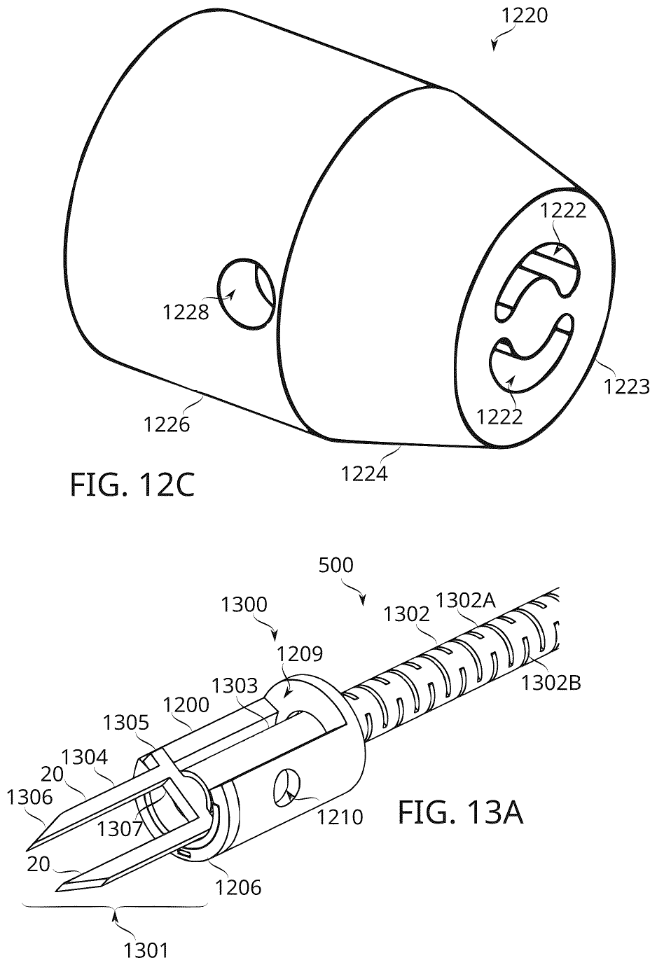

Reference is now made to FIGS. 12A-12B, which schematically represent a casing 1200 of a tip 1300 of a valve annuloplasty device 500, according to some embodiments of the present disclosure. For example, casing 1200 is provided as a component of the valve annuloplasty device 500 of FIGS. 7A-7E, and operates in conjunction with a support 702.

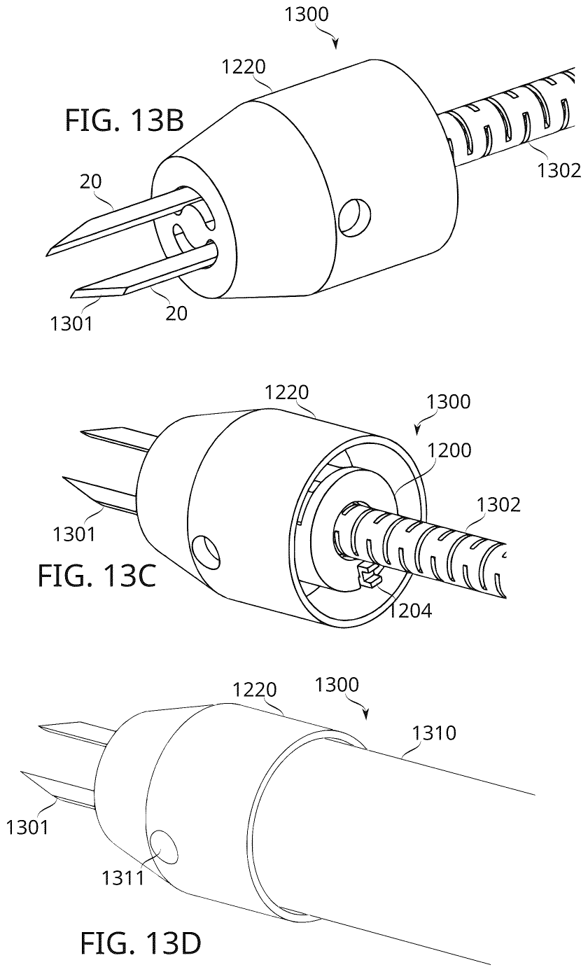

In some embodiments, casing 1200 comprises a generally cylindrical body 1205, a wall of the cylindrical body 1205 having an opening 1209 open along one side to an angular extent which corresponds (e.g., as described in relation to FIG. 13A) to an angular extent through which pins 20 (e.g., pin electrodes) are rotatable when mounted therewith. Body 1205 comprises non-conductive, and preferably heat-resistance material, e.g., PEEK or another a polymer material.

Also provided, in some embodiments, is a sensing electrode 1206, electrically coupled to a sensing circuit (e.g., sensor controller 36 of FIG. 5A) on a proximal side of valve annuloplasty device 500 (operating, e.g., as further described in relation to FIG. 13A). In some embodiments, sensing electrode 1206 is formed in the shape of a C-ring. In some embodiments, the electric coupling of sensing electrode 1206 is via a connection junction 1208 to a conducting strip 1207 (bridge) and connector 1204 leading therefrom. The connection junction 1208 may be secured, for example, using laser welding. Optionally sensing electrode 1206 and conducting strip 1207 comprise stainless steel material. Conducting strip 1207 may be positioned within a slot of body 1205.

In some embodiments, sensing electrode 1206 includes one or more portions 1206A, 1206B, each with a surface positioned where it comes into contact with pins 20 at some position(s) within their range of rotational and/or longitudinal travel.

In some embodiments, casing 1200 includes one or more cavities 1210, sized and shaped to house a portion of a radiopaque marker 1311 (e.g., as shown in FIG. 13D). In some embodiments, two cavities 1210 are diametrically positioned on the circumference of casing 1200. Reference is now made to FIG. 12C, which schematically represents a housing 1220 of a tip 1300 of a valve annuloplasty device 500, according to some embodiments of the present disclosure. For example, housing 1220 is provided as a component of the valve annuloplasty device 500 of FIGS. 7A-7E, corresponding to housing 505, including curved slot apertures 1222 in distal face 1223 corresponding to slot apertures 702. The curved slot apertures 1222 define tracks along which pins 20 of pin element 1301 move. The width of the slit apertures 1222 may be sized to the width of the pins 20 (e.g., about 0.4 mm), thus providing some mechanical support to them which helps prevent them from being snapped off by stress focusing at the location where they join the base 1307 (FIG. 13A). They are preferably long enough, however, that they do not limit the rotational movement by applying force to the pins 20 when they are at their fully rotated positions. Instead, force is taken up by interference of a portion of base 1307 (e.g., contract region 1305) with casing 1200.

Also shown are distally tapering body portion 1224, cylindrical body portion 1226, and cavity 1228. Optionally, cavity 1228 is one a of a plurality of cavities 1228; e.g., two cavities, diametrically placed on the circumference of housing 1220. In some embodiments, the cavities 1228 are positioned where a radiopaque marker 1311 inserted through cavity 1228 also inserts into a cavity 1210 of casing 1200, acting as a pin to secure them to each other.

In some embodiments, housing 1220 comprise a polymeric and electrically isolating material, preferably heat resistant; e.g., PEEK.

Reference is now made to FIG. 13A, which schematically represents a partially assembled tip 1300 of a valve annuloplasty device 500, according to some embodiments of the present disclosure. For example, tip 1300 corresponds to a tip 501 of the valve annuloplasty device 500 of FIGS. 7A-7E.

The partially assembled components shown include casing 1200, pin support 1303 (e.g., corresponding to support 703 of FIG. 7A-7E), pin element 1301, and connection cable 1302. Connection cable 1302 is flexible; e.g., an outer casing 1302A of connection cable 1302 slotted with slots 1302B to increase its flexibility. In some embodiments, outer casing 1302A comprises stainless steel, optionally provided with an electrically isolating sleeve such as polymer shrink tubing.

Pin element 1301, in some embodiments, comprises a plurality of pins 20, each comprising a pin body 1304 and sharpened tip 1306. Pin body 1304 may be quadrilateral (e.g., square) in cross-section. Pins 20 may be joined together through a base 1307, which connects them to pin support 1303. Base 1307 is electrically connected to the proximal side of annuloplasty device 500 through one or more electrical conductors of connection cable 1302.

The pins 20 may be electrically connected to each other, or they may be separately electrically connected through connection cable 1302. Optionally, connection is through the (electrically conductive) body of outer casing 1302A, e.g., via a connection made using laser welding. Through their electrical connection(s) pins 20 are optionally configured as pin electrodes 20 to deliver, e.g., RF, microwave frequency and/or electroporation energy to tissue; for example in a monopolar or bipolar configuration. As shown, pin element 1301 also comprises a contact region 1305. Optionally, all of pin element 1301 is constructed of a single metallic piece, e.g., a piece stamp-or laser-cut from sheet stock, or a 3-D printed component. In some embodiments, pin element 1301 comprises tantalum, which provides both electrically conductive and fluoroscopically radiopaque properties.

Pin support 1303 is rotatable relative to casing 1200, and also movable relative to casing 1200 along a proximal-distal axis; for example by movements of connection cable 1302 and/or a mechanical element extending within outer casing 1302A. Since contact is stopped by mechanical interference with the base 1307 of pin element 1301, the more fragile tip regions of the pin element (e.g., pin bodies 1304) are protected from over-torquing, which could lead to them bending, slipping out of tissue, and/or otherwise adverse events.

These movements change the nature of the electrical contact which contact region 1305 has with sensing electrode 1206. In the example shown, contact region 1305 makes electrical contact with sensing electrode 1206 when pins 20 are fully advanced and maximally rotated to one or the other sides of opening 1209.

This allows sensor controller 36 to detect these particular conditions, and optionally to indicate them, for example by illumination of an LED, and/or another indication such as an audible, haptic, and/or other type of visually presented indication. Optionally, one or more other positions on case 1200 are electrically interconnected with sensing electrode 1206, allowing other positions to be detected, such as one or more fully withdrawn positions of pin element 1301. Among a plurality of positions which may be detected through the same sensing apparatus (and indicated in the same way), the operator can determine which is actually relevant, since they know, e.g., to which side they have turned the pin element 1301, and/or whether they are withdrawing or advancing pin element 1301.

In some embodiments, the sensing apparatus itself is configured to distinguish different positions. For example, different contact regions on casing 1200 are connected to sensor controller 36 through resistances of different values. Optionally, a variable resistor-type configuration is provided, wherein electrical contact between pin element 1301 and casing 1200 is made through a resistive element, e.g., an inner lining of casing 1200 comprising polymer-embedded carbon particles, a resistive paste, or another material. This potentially allows graded detection of response to inputted movement commands, e.g., to allow distinguishing and/or evaluating partial rotations and/or distances of pin advancement along the proximal/distal axis.

Reference is now made to FIGS. 13B-13C, which schematically illustrates partially assembled tip 1300 of a valve annuloplasty device 500 with housing 1220 added, according to some embodiments of the present disclosure. As shown, pins 20 of pin element 1301 are fully extended. From the proximal-side view of FIG. 13B, it may be seen that connector 1204 is exposed where it can be connected to during final assembly.

Reference is now made to FIG. 13D, which schematically illustrates tip 1300 of a valve annuloplasty device 500, now with catheter casing 1310 added, according to some embodiments of the present disclosure. Optionally, radiopaque element(s) 1311 are used as pins to interconnect housing 1220 and casing 1200, and optionally also catheter casing 1310 (e.g., through holes in the casing material). As appropriate, catheter casing 1310 may be adhered to housing 1220 and/or casing 1200, e.g., using adhesive glue and/or melting. It should be noted that the assembly shown allows connector cable 1302 and catheter casing 1310 to rotate with respect to each other, with the result that pin element 1301 is rotatable relative to housing 1220. In some embodiments, the assembly includes steering elements (e.g., longitudinally elongated elements, not shown) which connect to one or more sides of the assembly, and can be shortened or otherwise manipulated to divert the orientation of the device tip.

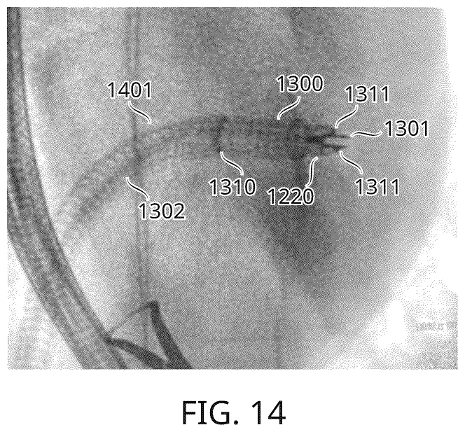

Reference is now made to FIG. 14, which presents a fluoroscopic (X-ray) image of a distal tip 1300 of a valve annuloplasty device 500 in situ (that is, within a chamber of a heart), according to some embodiments of the present disclosure. Indicated particularly in the image are pin assembly 1301, radiopaque markers 1311 (two, for example), the position of housing 1220, and the passage of connection cable 1302 through catheter casing 1310 and guiding sheath 1401. For effective performance of an annuloplasty procedure, it is a potential advantage to include device features and/or methodological approaches which allow the operator to develop and maintain clear and unambiguous awareness of the relative position of device and tissue target. For example, the positions of radiopaque markers 1311 can be adjusted by rotation until they are shown at their widest (as shown) or narrowest distance, allowing the orientation of distal tip 1300 to be calibrated relative to one or more known and/or predefined fluoroscopic viewing angles (views). Optionally, the reference viewing angle is shifted according to which region of the annulus is being worked on. For example, different respective angles (e.g., different positions of a fluoroscope's “C” arm) are selected for different circumferential regions along a mitral valve annulus; e.g., different views for P1, P2, and/or P3 regions along a posterior side of the valve annulus.

### Operation of an Annuloplasty Device Using Rotational Distortion

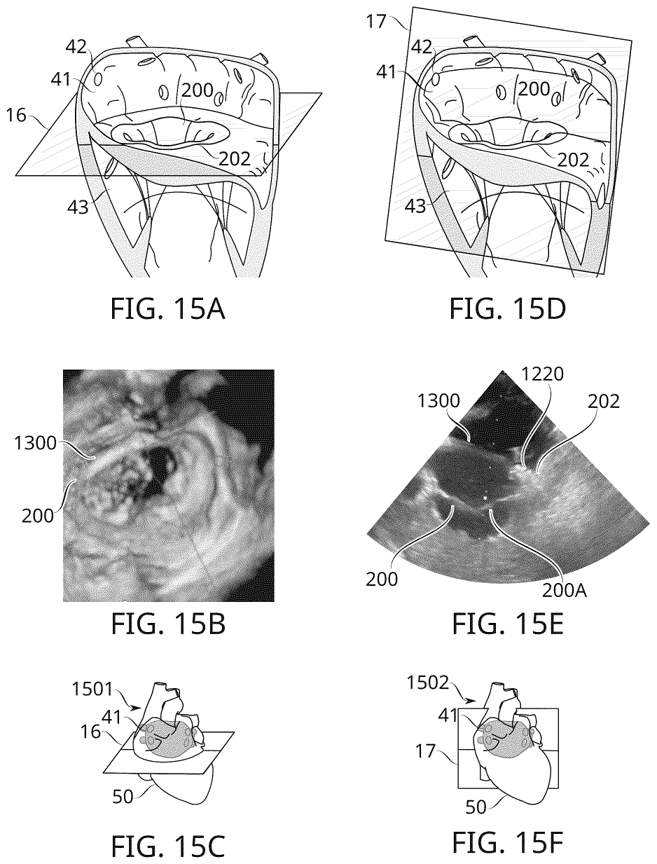

Reference is now made to FIGS. 15A and 15C, which schematically illustrate a horizontal (transverse) section 16 through a left side of a heart including left atrium 41 and left ventricle 43, according to some embodiments of the present disclosure. Also indicated (FIG. 15A) are positions of the foramen ovalis 42 (often used in minimally invasive heart procedures as a passage between the right and left atria), the mitral valve 200, and the mitral valve annulus 202. Icon 1501 of FIG. 15C provides a more overall view of the situation, with plane 16 crossing through heart 50 at a position near the bottom of left atrium 41 (shaded, and hidden behind heart 50 from this perspective).

Further reference is made to FIG. 15B, which presents a 3-D reconstructed ultrasound image which looks down at valve 200 and the horizontal (transverse) section 16 from a vantage point above it, according to some embodiments of the present disclosure. Also visible as a generally cylindrical body is distal tip 1300 of an annuloplasty device 500.

Reference is now made to FIGS. 15D and 15F, which schematically illustrate a coronal (frontal plane) section 17 through a left side of a heart including left atrium 41 and left ventricle 43, according to some embodiments of the present disclosure. Also indicated (FIG. 15D) are positions of the foramen ovalis 42, the mitral valve 200, and the mitral valve annulus 202. Icon 1502 of FIG. 15F provides a more overall view of how plane 17 crosses through heart 50.

Further reference is made to FIG. 15E, which presents a planar ultrasound image providing a coronal cross-section (corresponding to coronal section 17) of valve 200 (e.g., showing the valve leaflets 200A), according to some embodiments of the present disclosure. Also visible as a longitudinally elongated body is distal tip 1300 of an annuloplasty device 500, crossing over the valve to position housing 1220 in contact with valve annulus 202.

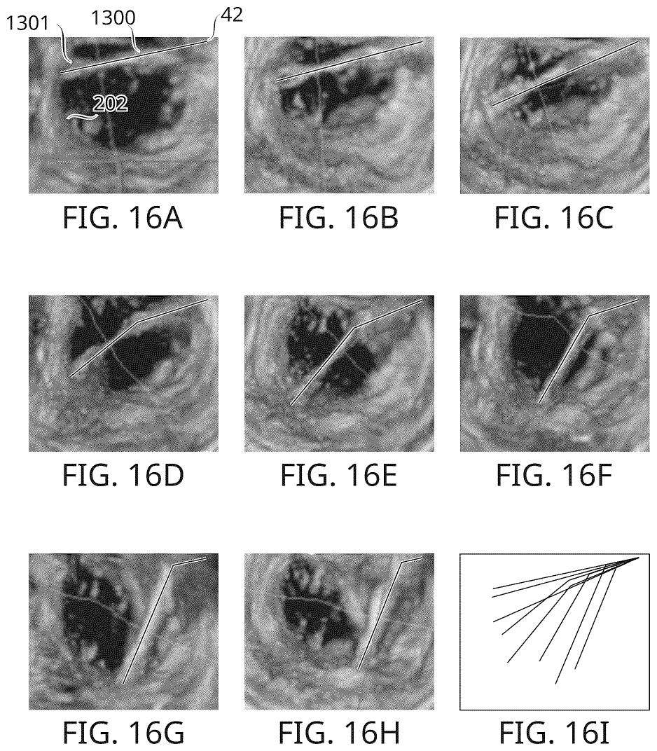

Reference is now made to FIGS. 16A-16H, which present a sequence of 3-D reconstructed ultrasound images obtained at different positions and/or articulations of tip 1300 as it moves pin element 1301 around a circumference of mitral valve annulus 202, according to some embodiments of the present disclosure. Tip 1300 has entered the left atrium from the position of foramen ovalis 42. The approximate position and longitudinally elongated shape followed by tip 1300 in each image is indicated by a white-outlined black line. Reference is also made to FIG. 161, which overlays the various positions of FIGS. 16A-16H in a single image.

At each such position, device tip 1300 was operated to insert pins 20 into the tissue of valve annulus 202, twist it, and then induce plastic deformation by delivery of energy (in this case, by delivering RF energy through the pins 20 acting as pin electrodes; optionally microwave and/or electroporating energy is provided).

It may be appreciated that although the general situation may be apprehended from the kinds of ultrasound and/or fluoroscopic images shown and/or explained in FIGS. 14-161, there remains a potential problem for determining precisely where the targeted valve annulus tissue is, and how tip 1300 should be operated so as to manipulate the targeted valve annulus tissue appropriately, and deliver structurally disruptive energy through tip 1300.

### Operational Features of an Annuloplasty Device Using Rotational Distortion

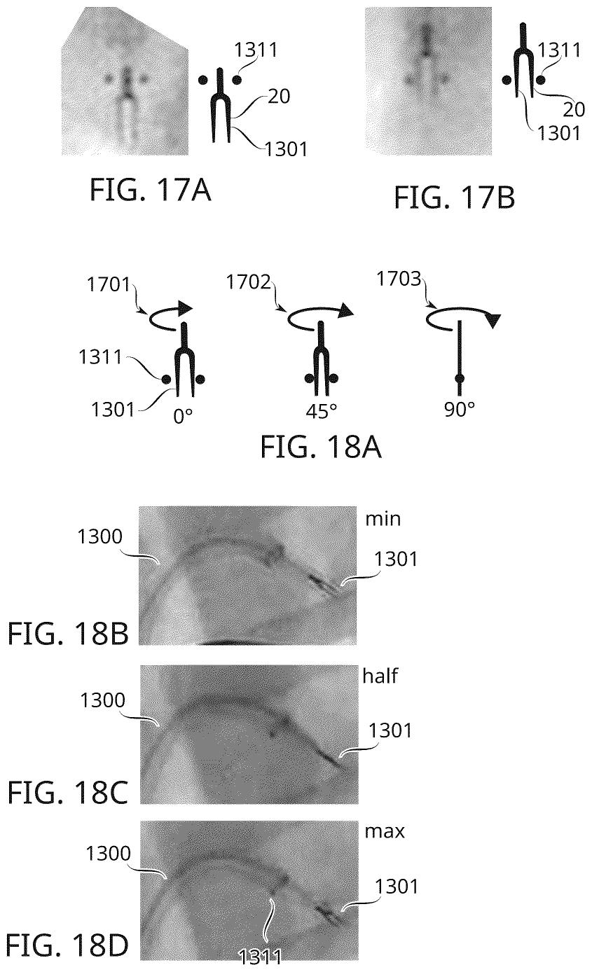

Reference is now made to FIGS. 17A-17B, which illustrate fluoroscopic (X-ray) images in situ (that is, within a chamber of a heart) of a pin assembly 1301 (which may comprise a radiopaque material such as tantalum) in relation to radiopaque markers 1311, as well as schematic drawings of the same, according to some embodiments of the present disclosure. FIG. 17B images a device configuration with pin assembly 1301 fully withdrawn into a housing such as housing 1220 of FIG. 12C. This configuration can be identified by the relative location of radiopaque markers 1311 directly alongside the pins 20. FIG. 17A, images the pins 20 in an extended position, advanced well past the longitudinal position of the radiopaque markers 1311. Reference is now made to FIG. 18A, which schematically represents views of pin assembly 1301 and radiopaque markers 1311 at different rotational positions, according to some embodiments of the present disclosure. Further reference is made to FIGS. 18B-18D, which show X-ray images of a distal tip 1300, including with views of pins 1301 from different rotational angles, according to some embodiments of the present disclosure.

In FIG. 18A, the view at angle 1701 shows pin assembly 1301 and radiopaque markers 1311 at their widest separation (viewed flat on). The view associated with rotation angle 1702 adds 45° of rotation, and the view associated with rotation angle 1703 adds 90° of rotation. With an additional 45° of rotation, the view would again look like that associated with rotation 1702.

The view of angle 1702 corresponds, in some embodiments, approximately to the oblique angle views seen in FIGS. 18B and 18D, while the view of angle 1703 corresponds to the rotation of angle 1703. Accordingly, the sequence of images from FIG. 18B through 18D corresponds to an approximately 90° rotation, from one side to the other. Although ambiguous in a static image, the two 45° angles of rotation can be distinguished by observation through time of the transition, and/or by knowing how much and in what direction control has been exerted to generate the positional transfer. It is noted that opaque markers 1311 are shown in this embodiment at somewhat more proximal position relative to pin assembly 1301 than is shown in FIGS. 18A-18D.

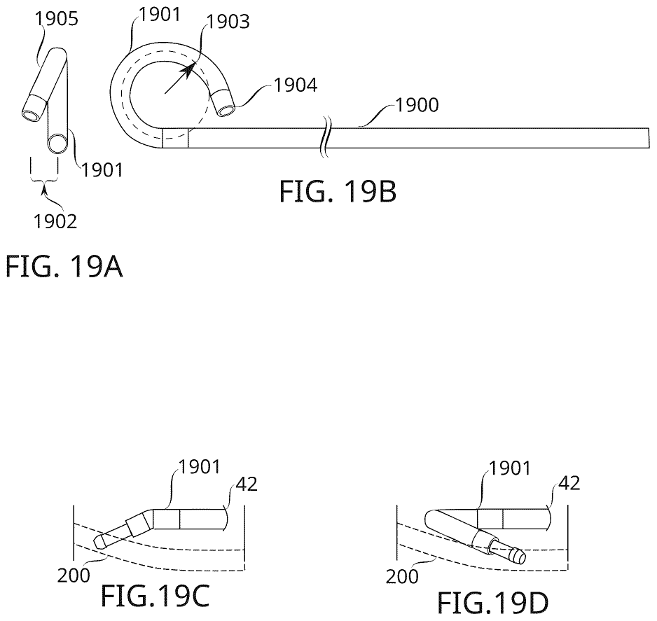

Reference is now made to FIGS. 19A-19B, which schematically represent an overtube 1901 predisposed to assume a compound bending which adds both curvature and displacement out-of-plane, according to some embodiments of the present disclosure. In some embodiments, the predisposed bending is assumed upon removal of confinement from overtube 1901, e.g., confinement by a further outer tube, e.g., tube 1900. Alternatively, in some embodiments, the shape shown is the “extreme” bending of overtube 1901 with fully actuated steering. When steering is not actuated (or oppositely actuated), overtube 1901 straightens. Optionally, the overtube 1901 is biased to bend, and steering actuates to bend it further, or optionally to straighten it away from the biased bending. The minimum radius of curvature may be, for example, the radius 1903. In some embodiments, the catheter device itself is configured to be steerable in the same manner as described for the overtube 1901.

The view of overtube 1901 of FIG. 19A represents an end-on view of the curled portion, including segment 1905, which is predisposed to bend out of plane (that is, out of a plane of the curling and or assume a helical shape) as it curls by distance 1902. Optionally, curling and/or out of plane displacement is at least partially assumed upon extending overtube 1901 from confinement by segment 1900, which may be provided as a further overtube. Additional (or reduced) curling and/or out of plane displacement is optionally provided by steering inputs. Alternatively, segment 1900 may be provided as part of overtube 1901, with the curling being controlled essentially wholly by steering inputs. Steering inputs may be provided, for example, via a longitudinally elongated element (not shown) which attaches to tip 1904, and extends along the body of tube 1901 and back along segment 1900. The element is attached to tip 1904 such that tension placed on the element induces steering motions as shown. In particular, the elongated element may wrap partially around the circumference of segment 1905 as it extends toward tip 1904, so that when it is longitudinally shortened, it pulls tip 1904 out of plane through distance 1902, as well as inducing curling up to main radius of curvature 1903. It may be noted as well that progressive coordination of main curvature and out-of-plane bending has potential advantages for addressing a generally saddle-shaped valve annulus such as the mitral valve, for example as discussed in relation to FIG. 19C.

In some embodiments, tip 1904 of overtube 1901 is also steerable separately from the overall bending, at least in part, e.g., to allow adjustment of out of plane distance 1902.

It should be noted that the curvatures shown and discussed in relation to FIGS. 19A-19B are potentially well suited to shallow angles of incidence upon the circumference of the valve annulus, with respect to angle above or below the (local) plane of the valve. For example, the angle may be within 60° of this plane, within 45° of this plane, within 35°, within 30°, or within 25°. In some embodiments, the angle is within 30°-50° of the local plane of the valve, optionally lower, and preferably not higher. Insofar as the valve may be non-planar in shape (e.g., saddle-shaped), the “local” plane of the valve refers to a plane which includes (e.g., includes with least error) some abbreviated portion of the overall valve annulus. The shallow angle of approach provides potential advantages for avoiding valve leaflet injury and/or reproducible positioning against and/or engagement with valve annulus tissue.

Reference is now made to FIGS. 19C-19D, which schematically represent overtube 1901 (or a catheter itself, similarly configured for steering), in relation to a roughly saddle-shaped valve annulus 200, according to some embodiments of the present disclosure. In FIG. 19C, a slight steering input (tensioning) leads to a relatively slight circular bend, as well as a relatively slight out-of-plane bending, away from the position of foramen ovalis 42. For example, 10-20 mm of out-of-plane displacement may be needed to reach the valve tissue. In FIG. 19D, a strong steering input (more tensioning) leads to a relatively tight circular bend, as well as a relatively large amount of out-of-plane bending, away from the position of foramen ovalis 42. For example, 35-45 mm of out-of-plane displacement may be needed to reach the valve tissue. In some embodiments, the position of FIG. 19C corresponds to accessing region P1 of the mitral valve annulus (distal portion of the posterior valve annulus) for treatment. In some embodiments, the position of FIG. 19D corresponds to accessing region P3 of the mitral valve annulus (proximal portion of the posterior valve annulus) for treatment.

### Bio-Impedance Heuristics

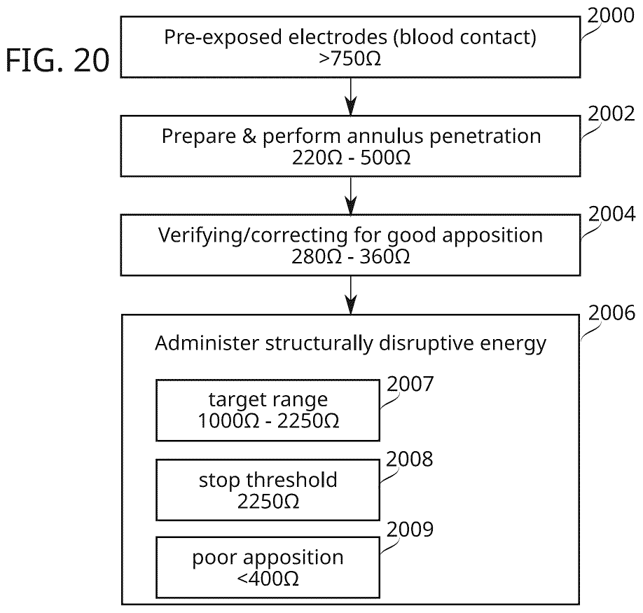

Reference is now made to FIG. 20, which is a schematic flowchart representing targeted and/or expected thresholds and/or ranges of bio-impedance in Ohms (Ω) for various phases and/or conditions during an annuloplasty procedure, according to some embodiments of the present disclosure.

The values shown were developed experimentally while using pins 20 which are also electrodes delivering the structurally disruptive energy at RF frequencies. The pin electrodes were constructed of stainless steel, with square cross-sections measuring about 0.4 mm×0.4 mm. However, values with other (electrically conductive) electrode materials such as tantalum are expected to be similar. Values apply to a distance between the electrodes in a range of about 1-2 mm, and a distance of penetrating tissue of about 4 mm. Furthermore, the values shown are optionally used with operational parameters comprising delivery of 8-10 Watts of RF power, over a time of about 8-25 seconds. In some embodiments, impedance measurements are performed at a frequency in a range of 25-75 kHz (e.g., 50 kHz). Frequency sampling is preferably performed above the corresponding Nyquist frequency; e.g., at about 2 Mhz. With a 50 KHz measurement frequency, this allows, for example, the use of 60 microseconds of data (120 samples) to capture 3 whole waveforms, which potentially helps to screen out noise. In some embodiments, results are displayed at intervals of about 1 second; optionally faster or slower.

At block 2000 is shown a time during which the electrodes, in their pre-exposed state (withdrawn into a housing 1220, for example), are in blood contact, but not tissue contact. A typical bio-impedance measured in this condition was larger than about 750 Ω.

At block 2002, bringing the device tip into contact with the tissue and initiating electrode insertion, results in a measured drop in bio-impedance, determined to be typically to a range of about 220 Ω to 500 Ω while insertion is underway. In some embodiments, this range is used as an indication that the device tip is positioned where pins (e.g., pin electrodes) may be extended to insert into annulus tissue. Optionally the upper value of this expected range is lowered, e.g., to 400 Ω; potentially increasing a reproducibility of procedure results, though also potentially increasing a need for longer and/or repeated attempts to position the device tip. If the expected range is not generally achieved and/or maintained during insertion, it may indicate failure to penetrate and/or loss of penetration.

At block 2004, final annulus penetration with apposition appropriate to initiate structural disruption (as the pins 20 extend into the tissue) typically (but not invariably) resulted in a narrowing in bio-impedance from the more general range associated with insertion as such. Depending on initial conditions, this was observed to result variably in a slight rise or slight fall from the initial range, typically to within a range of 240 Ω-360 Ω. In some embodiments, the impedance is in a range between 200 Ω-400 Ω. Optionally, achieving impedance in this range is used as gating condition for further operations, and/or as confirmation of “good apposition” of the device to tissue, with pins inserted, so that the tissue may be manipulated (e.g., distorted by rotation), potentially without loss of electrical contact and/or damage to the device pins. Particularly if the impedance is lower, it may indicate that the extended pins are exposed to each other across an open blood-filled volume, rather than through tissue. Pressing the device forward may lead to a rise in impedance, potentially indicative of corrected apposition. Optionally the upper value of “good apposition” range is lowered, e.g., to 320 Ω; and/or the lower value of this range is raised, e.g., to 280 Ω. Such range reduction potentially increases a reproducibility of the procedure, though also potentially increasing a need for longer and/or repeated attempts to position the device tip.

Block 2006 (together with its sub-blocks) represents some potential bio-impedance states resulting consequent to the application of structurally disruptive energy. Block 2007 indicates a target range of about 1000 Ω-2250 Ω, corresponding to a range of bio-impedances which correspond to a successful administration of structurally disruptive energy. Above 2250 Ω (block 2008), in the example shown, administration of structurally disruptive energy is optionally stopped, even if the planned time of power delivery (e.g., within the range of 15-30 seconds has not been reached). Optionally, the stopping threshold is selected from a value within a range of 1800-2500 Ω. The upper value of this range is optionally lowered; for example, to 2400 Ω, potentially leading to an increased margin of safety. Additionally or alternatively, the lower value of this range is optionally raised, for example, to 2000 Ω, potentially leading to decreased occurrence of unnecessary system self-shutdowns. In some embodiments, stopping is automatic upon reaching a selected stopping threshold. Optionally, automatic stopping is coupled to a feature of the time-course of impedance change. For example, an impedance change which is sudden relative to the ordinary time course of structural disruption (a few hundred milliseconds instead of several seconds) is optionally filtered from triggering stoppage until a more physiologically plausible period of time has elapsed. Block 2009 represents bio-impedance remaining at (or suddenly reverting to) a level less than about 400 Ω during delivery of structurally disruptive energy. This optionally is taken as an indication of poor apposition or loss of apposition, notwithstanding what the history of other bio-impedance indications may have indicated.

In some embodiments, bio-impedance measurements are used in connection with imaging and/or another measurement type in order to verify that device operation is proceeding according to the expected sequence of events.

### Methods of Operating Annuloplasty Devices

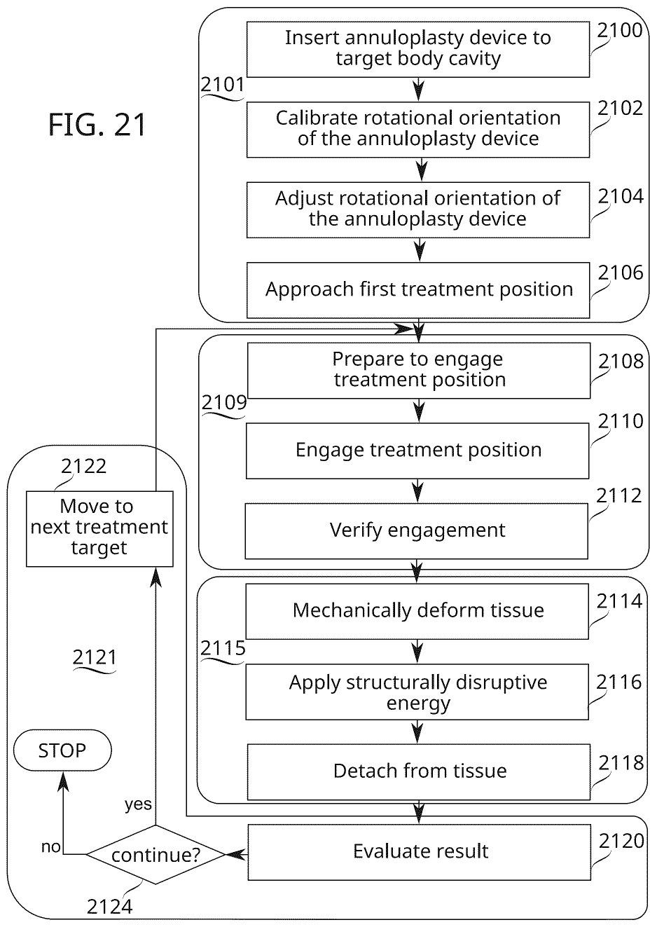

Reference is now made to FIG. 21, which is a schematic flowchart representing methods of operating a valve annuloplasty device, according to some embodiments of the present disclosure. In some embodiments, the valve annuloplasty device operates according to principles of tissue shrinking by a combination of twist/torsion (e.g., as described in relation to FIGS. 5A-11 herein) and the application of structurally disruptive energy.

In summary, the method comprises the larger-block enclosed operations indicated: block 2101 comprising initial device positioning and optional calibration of positioning; block 2109, comprising engagement of the annuloplasty device to a treatment position; block 2115 comprising the deformation and structural disruption (plastic deformation) of a treatment position; and block 2121, comprising evaluation of results and (while relevant) continuation on to the next treatment position. The various sub-blocks are provided as non-limiting examples of categories of activities (with further examples described below) which may take place within each of the larger blocks. At block 2100, in some embodiments, the valve annuloplasty device is inserted into a body cavity adjacent to the valve to be treated, e.g., inserted to a left atrium via a fossa ovalis of an interatrial septal wall, for treatment of a mitral valve. In some embodiments, another heart valve is treated; for example, a tricuspid valve.

At block 2102, in some embodiments, a rotational orientation of the valve annuloplasty device is calibrated. This is of particular relevance to the pin-twisting embodiments of FIGS. 5A-11, but also optionally applies, e.g., to the vacuum-using embodiments of FIGS. 1A-4C, when the lesions formed are longer in one dimension than in another, and/or configured to pinch up tissue preferentially along a particular axis.

In some embodiments, calibrating the rotational orientation of the valve annuloplasty device comprises rotating the annuloplasty device under imaging visualization, until a configuration of the device (as viewed from the angle of visualization) matches a known rotational state. In some embodiments, this comprises rotation of a portion of the device comprising at least two radiopaque markers until the markers reach a maximum or minimum distance in a fluoroscopic image which visualizes them. Optionally, the radiopaque markers comprise pins which are themselves used in mechanically manipulating and/or energetically modifying the tissue of the analysis. Optionally, the radiopaque markers comprise elements added to a tip of the device in a known and/or determinable relationship to the orientation of the pins.

In some embodiments, the calibration uses another method; for example, an ultrasound scattering and/or reflecting pattern which returns a distinctive signal when oriented toward the position of the ultrasound detector and/or ultrasound energy source.

Where fluoroscopic and/or ultrasound visualization is readily available, directly imaging the annuloplasty device itself may provide a preferable method of determining device orientation, as providing potential advantages for directly and intuitively understanding how the annuloplasty device is positioned. However, it is a potential advantage to calibrate and/or monitor device orientation using a non-imaging method, e.g., to reduce procedure complexity and/or reduce radiation exposure.

Using such methods, orientation of the annuloplasty device itself is optionally detected directly. Optionally, orientation of another element (e.g., an overtube or a specialized measurement device) may be determined, and this information transferred via known spatial and/or additional measurement constraints to apply the annuloplasty device. The indirect methods have the potential advantage of not adding sensor bulk directly to the annuloplasty device, although it is noted that sensing element(s) may optionally be placed somewhat proximal of the tip itself, where there is potentially more available room. Insofar as the annuloplasty device itself is directly performing the sensing and/or being sensed, there are potential advantages for real-time and/or more clearly localized sensing of device orientation and/or position.

Non-imaging sensing principles for determining device orientation optionally include, for example, one or more of magnetic field orientation sensing, acceleration sensing (e.g., gravimetric sensing), pressure sensing (e.g., of blood, for example, differential pressures on opposites sides of a sensing head), flow sensing (e.g., of blood), and strain sensing (e.g., to determine twist along a device). Additionally or alternatively, physical constraints may be used to help determine orientation, and/or assist in transferring known orientation information about one element to the annuloplasty device. As examples:
In some embodiments, a magnetic field emitter and/or detector associated in a known and/or determinable orientation with respect to pins of the annuloplasty device is used, with the corresponding detector/emitter placed elsewhere to sense/activate the pin-associated emitted/detector. For example, a guide wire comprising one or more miniature magnets and/or electromagnets is optionally placed in the circumflex and/or the coronary sinus around the valve annulus. This may be detected by the device tip (e.g., using a MEMS Hall effect detector), and potentially even be used to magnetically attract the device tip towards the annulus.

In some embodiments, orientation is determined at least in part by noting a direction of flexion when a steering degree of freedom is operated to move the tip (i.e., bend it). Determination of the direction may be based on a change in a measured parameter (e.g., a direction of acceleration determined with respect to gravity), and/or association with physical constraints on the movement itself (e.g., steering may be associated with limitations of distance in some directions more than in others).

In some embodiments, it may be convenient to directly determine the orientation of the overtube, rather than the annuloplasty device itself. For example, the overtube orientation may be known by imaging and/or tracking used to maneuver it into position, after which imaging can optionally be discontinued or reduced, at least insofar as the overtube orientation can be transferred to information about the orientation of the annuloplasty device (e.g., as next described). In some embodiments, the history of how the overtube was manipulated to get it into position (e.g., by steering and/or twisting it) is tracked. The overtube may (e.g., because it larger) be relatively resistant to twisting around its longitudinal axis, its general position in and course through the body known, and the orientation of a proximal portion of the overtube known. Optionally, the overtube actually is susceptible to twisting, but calibrated sensing (e.g., via a strain gauge arrangement such as an optical fiber strain gauge) is used to determine the amount of twisting.

As a means of transferring orientation information, in some embodiments, an overtube used with the annuloplasty device is provided with an obstruction (e.g., of its distal aperture and/or another location proximal to its distal aperture) which generates a non-circular (“keyed” or “warded”) shape through which the annuloplasty device passes (e.g., the shape is indented from circularity on one or more sides). The non-circular shape is selected so that a cross-sectional profile of the annuloplasty device is only able to pass the constriction in one or more particular matching orientations. Optionally, a detent-type interaction between the overtube and the annuloplasty device is used (e.g., a bump on the inner lumen of the overtube and a receiving divot on the annuloplasty device), resulting in a mechanical “hesitation” which an operator can feel at some orientation or orientations, and no such hesitation at other angles. In some embodiments, electrical sensing (e.g., contact and/or resistive film based) is used to determine relative orientations of the overtube (acting as a frame of reference) and the annuloplasty device.

Optionally, a sensing probe advanced through the overtube before use of the annuloplasty device determines the overtube orientation, for example based on any of the sensing principles mentioned above. This information is transferred to help determine the orientation of the annuloplasty device when it itself is inserted, e.g., transferred to the overtube orientation via a “keying” or detent-type mechanism, and from there to the annuloplasty device itself.

At block 2104, in some embodiments, orientation of the annuloplasty device is adjusted from the known/calibrated position to a targeted position, optionally and as appropriate to the intended orientation of the device pins relative to the valve annulus. This is optionally done “blindly” by an offset of a fixed amount from the calibrated orientation. Optionally, sensing feedback continues to be provided during the adjustment. The preferred orientation in the targeted position optionally depends on device type. For example vacuum-type devices which produce a lozenge-shaped lesion (e.g., FIGS. 1A-4C) may be preferentially oriented with a long axis of the lozenge oriented perpendicularly to the circumferential direction of the valve annulus. Whatever the orientation (if any) of the lozenge-shape portion of the lesion, a pinching-type vacuum-type device may be preferentially oriented so that the direction most shortened by the pinching is the circumferential direction. The pin-type “twisting” devices of FIGS. 5A-11 may be preferably oriented with an axis extending between the two pins (e.g., their tips) oriented oblique to the circumferential direction of the valve annulus. For embodiments which combine vacuum attachment with twisting deformation, the preferred orientation may be oblique as for the pin-type twisting devices. This also applies to embodiments which operate by vacuum attachment with pins also used, e.g., to prevent slippage and/or encourage deeper lesion penetration by acting as energy delivering electrodes.

In some embodiments, once a rotational orientation is calibrated at block 2102 by a first method, device and/or principle, rotational position may be tracked further by another method, device and/or principle. For example, rotation of an electrode relative to a resistive film may be measured. In some embodiments, the electrode is placed on a surface of the annuloplasty device where it contacts a portion of the interior lumen of an overtube lined with a resistive film, and changes in resistance are used to track orientation changes. Additionally or alternatively, the operator is enabled to determine orientation, at least approximately, by setting a marker on a handle of the annuloplasty device in the “calibrated” position, and thereafter noting offsets from this position, e.g., relative to an overtube or to the general surroundings.

Rotational adjustment operations optionally comprise adjusting the angle of incidence above or below the plane of the valve which the annuloplasty device assumes against tissue of the valve annulus.

At block 2106, in some embodiments, the annuloplasty device is brought to a first selected position around the valve annulus to begin a treatment. Optionally, operations to evaluate the orientation of the device (e.g., as described in relation to blocks 2102-2104) are determined during operations of this block.

In some embodiments, the operations of block 2106 are performed under live image-assisted guidance; e.g., ultrasound and/or fluoroscopic observation, for example as described in relation to any of the images shown in of FIGS. 14-18D. Additionally or alternatively, in some embodiments, non-imaging measurements are performed to determine and/or confirm device positioning. For example, bio-impedance measurements are performed, e.g., as described in relation to FIG. 20.

In some embodiments, a shape of the relevant anatomy of the valve to be treated is known from previous imaging, e.g., pre-procedural imaging, or imaging at an earlier stage of the current procedure. The anatomical shape may be determined, for example, by segmentation of a (3-D) MRI or CT image. Optionally, the shape is determined from 2-D fluoroscopic images, e.g., based on images of the distribution of an injected contrast agent, optionally with the 3-D shape being reconstructed from stereographic constraints on images taken from different directions. An insertion position of the annuloplasty device into the vicinity of the valve may be determined, e. g., from image-based detection of the position of a fossa ovalis relative to the valve, and/or by noting the position of the annuloplasty device at the time of procedure via brief imaging.

Insofar as the annuloplasty device assumes a predictable shape in response to steering inputs and/or longitudinal device advance, the amount of such control inputs (optionally measured by suitable encoders, in some embodiments) is optionally used to infer a position of the device. In some embodiments, the general results of these inferences are optionally confirmed and/or corrected under guidance from other measurements. Again, for example, this correction may be according to bio-impedance heuristics as described in relation to FIG. 20. Monitoring time courses of changes in bio-impedance measurements as the annuloplasty device is manipulated potentially also assists, optionally, in finding treatment positions, e.g., manipulation can be performed to locate the device at a local minimum or maximum of measured bio-impedance. Other sensing options which may assist in device localization relative to nearby anatomy of the valve annulus include, for example, force sensing (e.g., of contact), flow sensing, and/or pressure sensing. Flow and pressure sensing potentially indicate conditions characteristic of blood movement near the periphery of the valve (and the valve annulus), compared to conditions characteristic of the high blood-flow regions nearer to the center of the valve. Potentially, flow and/or pressure sensing may distinguish regions separating leaflets (that is find and/or confirm the locations of leaflet boundaries).

In some embodiments, a device comprising one or more electrodes placed in (e.g., along) the coronary sinus is used to assist in localizing position of the annuloplasty device. This may operate based on electrical sensing by and/or of electrode-configured elements of the annuloplasty device such as the pins, and/or another electrode of the annuloplasty device tip, within the context of electrical fields transmitted by/to the electrode(s) within the coronary sinus.

Optionally, a model of the anatomical scenario (e.g., based on imaging and/or other data) is adjusted and/or recalibrated in response to sensed information. For example, it is adjusted according to locations where force is detected (e.g., by a contact force sensor on the tip of the annuloplasty device), measurements of bio-impedance, and/or changes in sensed fluid flow and/or pressure. Optionally, the anatomical scenario is adjusted and/or recalibrated in response to changes generated by the ongoing annuloplasty procedure which remodel the shape of the valve annulus, according to one or both of imaged changes, non-imaging sensor indicated changes, and/or expected effects of the procedure on valve shape.

At block 2108, in some embodiments, the annuloplasty device is suitably arranged so that it is ready to mechanically engage with tissue of the valve annulus beginning from a predictable insertion angle and/or to a predictable extent. This block emphasizes refinements of the general goal of block 2106 to find the starting treatment position, and may be performed simultaneously with it. For the twisting pin-type embodiments of FIGS. 5A-11, the emphasis is, in particular, on at least two parameters. First, there should be obtained a suitable apposition with (flat distal-face contact with) tissue of the valve annulus so that penetration by the pins 20, when it is actuated, occurs with the intended depth, e.g., 4 mm of penetration. The bio-impedance heuristics of FIG. 20 provide an example of a measurement which may be used to assess suitable apposition. Second, the apposition should be at a suitable angle. The suitable angle may be selected (e.g., pre-selected) by the operations of blocks 2102-2104. Optionally, there may be further adjustment, e.g., insofar as the manipulations of block 2106 resulted in a change to device orientation. The suitable angle, in some embodiments, comprises an axis extending between two pins of the annuloplasty device which is oblique to the circumferential direction of the valve annulus, e.g., oblique by about 45°, or otherwise, for example as described together with examples of pin placement herein.

With vacuum-type devices, apposition is expected to be enhanced upon the application of suction.

At block 2110, in some embodiments, pins of the annuloplasty device are advanced from their housing to engage the tissue with which the device is in apposition. In some embodiments, the pins are about 0.4 mm×0.4 mm in shaft cross-section, about 4 mm long, and sharp at the end so as to assist in the penetration of tissue.

With vacuum-type devices, application of suction occurs at block 2110.

At block 2112, in some embodiments, suitable engagement of the pins with tissue of the valve annulus is optionally verified, for pin-using embodiments. This may comprise, for example, testing of a heuristic associated with a measurement value, e.g., as described in relation to FIG. 20. Additionally or alternatively, fluoroscopic imaging of contrast agent injection may show that the pins are suitably embedded in a region which the contrast agent does not reach. Additionally or alternatively, ultrasound imaging potentially assists in confirming pin penetration. In some embodiments, a surface electrode (e.g., a flat electrode placed on a distal surface of the housing of the annuloplasty device tip) is used to take bio-impedance measurements which are evaluated to confirm that this surface maintains tissue contact. Evidence of such contact, together with evidence consistent with penetration, potentially increases confidence that good penetration has been achieved.

As noted: with vacuum-type devices, application of suction occurs at block 2110. Insofar as contact is enhanced by this, there may be noticeable effects on bio-impedance measurements (if used) and/or the amount of negative pressure developed within the device, and this is used for the verification, in some embodiments, at block 2112.

At block 2114, in some embodiments, the pins of the annuloplasty device are rotated (e.g., as described in relation to FIGS. 5A-11) to mechanically deform the tissue with which they are engaged. Being thin, the pins are delicate, so it is a potential advantage for them to be supported and/or protected by the device in various ways; e.g., with stops, sensing and/or suitably sized slots, as described in relation to FIGS. 12A-13D. An intended amount of rotation (twist imparted to the valve annulus) may be about 90°. In some embodiments, an intended amount of rotation is an angular value within the range of about 80°-115°.

In some embodiments, the correct amount of rotation is achieved when the pins of the device are rotated through their full available range, e.g., from a starting position against a first stop, to a final position against a second stop. In some embodiments, sensing (e.g., electrical sensing) at the tip is used to electrically signal at least one of these stopped positions, for example as described in relation to FIGS. 12A-13A. Optionally, more positions are sensed and/or discriminated, optionally discretely or continuously. Optionally, for some treatment positions, rotation is sub-maximal. This may allow, for example, a lower local introduction of distortion, with a tradeoff of more treatment positions being needed to achieve a same overall level of valve circumference reduction.

The operations of block 2114 are not necessarily performed for pure vacuum-type embodiments, although additional mechanical deformation may be introduced, e.g., by pulling against a bracing contact with the tissue, e.g., as described in relation to FIGS. 4A-4C. In some embodiments, vacuum-type embodiments can undergo twisting (with or without pins). This potentially blends characteristics of lesions produced by embodiments of FIGS. 1A-4C and 5A-11.

At block 2116, in some embodiments, structurally disruptive energy is delivered, while the pins mechanically distort the tissue they are inserted to. For example about 120 J are delivered, over a period of about 8-25 seconds. In some embodiments, a rate of energy delivery is in a range of 6-12 Watts. Superficial and/or penetrating (pin) electrodes are optionally used for energy delivery. In some embodiments, the total energy delivered is in a range of about 40 J to about 300 J. In animal trials, the inventors have found that these levels of power and energy are apparently effective and safe, e.g., at least to the extent that they produce no harm to the circumflex or coronary sinus in the vicinity of the lesioning. The inventors have also found that under ultrasound imaging, changes in the acoustic impedance of tissue are potentially detectable over the time course of the ablation. Accordingly, ablated regions may appear in ultrasound images as “white spots” of, e.g., increased acoustic reflectance, allowing judgement not only of the effectiveness of individual lesion placements, but also judgement of intervals and/or overall shrinkage. For example, two initial lesions may be placed at a distance from each other, and further lesions placed intermediate to them, gradually reducing the distance between the more extremely positioned lesions.

At block 2118, in some embodiments, the annuloplasty device is detached from tissue. Optionally, this includes full withdrawal of pins (if present) into their housing, in preparation for further movements.

At block 2120, in some embodiments, the result achieved at block 2116 is optionally evaluated, for example using bio-impedance heuristics, e.g., as described in relation to FIG. 20. Additionally or alternatively, a thermometer sensor (e.g., positioned on a front surface of the annuloplasty device tip housing) is used to verify that energy delivery resulted in an expected amount of heating. Evaluations are optionally performed before the release of block 2118, and/or both before and after. Vacuum-applied lesions may acquire a characteristic raised-up shape, potentially detectable in a bio-impedance or other electrical signal by slightly withdrawing the electrodes from the surface of the valve annulus and moving the tip back and forth over the bump. Mechanical interference with movement may also potentially be noticeable.

At block 2122, in some embodiments, the annuloplasty device is moved to the next targeted region for lesioning. Optionally, the movement is be accomplished under full image guidance, e.g., fluoroscopic and/or ultrasound guidance. In some embodiments, the movement is simplified by the design of the device, e.g., the device is simply advanced or withdrawn by a small amount to reach the new treatment position. Optionally, additional steering is provided to allow fine adjustments, which may be informed by sensing and/or modeling information, for example as described in relation to block 2106. Changes of device orientation may be introduced, e.g., as described and/or referenced in relation to block 2104.

When twisting (e.g., of pins) is to be performed again in the same (clockwise or counterclockwise) direction as before, the device rotation is reset. Alternatively, twisting direction may alternate. Optionally, movements between treatment sites are planned initially using dry-run survey movements of the device around the valve annulus, confirming at each stage of advance (during the survey) that conditions appear suitable for operation, and consistent with plans. Optionally, these movements are replicated during actual annuloplasty treatment. Since the valve is expected to change shape (e.g., be reduced) during the actual treatment, replication of movements may include adjustments (optionally predetermined shortening adjustments) to compensate.

Should it be determined that operations at a particular treatment position were unsuccessful, a treatment plan may be adjusted. If the issue was, for example, a sudden loss of engagement, a repetition may be in order. If there is difficulty achieving engagement, and/or repeated loss of engagement at some location, the plan of treatment positions may be altered suitably to compensate for the loss of a site.

As long as targeted treatment positions remain to be treated, the method continues (block 2124) with a repetition of the operations of blocks 2108-2122, or else stops.

### General

As used herein with reference to quantity or value, the term “about” means “within ±10% of”.

The terms “comprises”, “comprising”, “includes”, “including”, “having” and their conjugates mean: “including but not limited to”.

The term “consisting of” means: “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

The words “example” and “exemplary” are used herein to mean “serving as an example, instance or illustration”. Any embodiment described as an “example” or “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the present disclosure may include a plurality of “optional” features except insofar as such features conflict.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Throughout this application, embodiments may be presented with reference to a range format. It should be understood that the description in range format is merely for convenience and brevity and should not be construed as an inflexible limitation on the scope of descriptions of the present disclosure. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, from “1 to 4”, from “1 to 5”, from “2 to 4”, “from 2 to 6”, from “3 to 6”, etc. ; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween.

Although descriptions of the present disclosure are provided in conjunction with specific embodiments, it is evident that many alternatives, modifications and variations will be apparent to those skilled in the art. Accordingly, it is intended to embrace all such alternatives, modifications and variations that fall within the spirit and broad scope of the appended claims.

It is appreciated that certain features which are, for clarity, described in the present disclosure in the context of separate embodiments, may also be provided in combination in a single embodiment. Conversely, various features, which are, for brevity, described in the context of a single embodiment, may also be provided separately or in any suitable subcombination or as suitable in any other described embodiment of the present disclosure. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, to the same extent as if each individual publication, patent or patent application were specifically and individually noted when referenced that it is to be incorporated herein by reference. In addition, citation or identification of any reference in this application shall not be construed as an admission that such reference is available as prior art to the present disclosure. To the extent that section headings are used, they should not be construed as necessarily limiting. In addition, any priority document(s) of this application is/are hereby incorporated herein by reference in its/their entirety.

1. An annuloplasty device configured to deliver structurally disruptive energy to a tip terminating a distal end of a catheter body of the device, the annuloplasty device comprising:
a housing of the tip extending between a proximal side connected with the catheter body, and a distal side; and
a plurality of pins, each pin:
extending distally from a position anchored to the tip within the housing, and
sized and sharp to penetrate collagenous tissue of a valve annulus of a human heart;
wherein the pins are configured to move by rotation relative to the valve annulus with sufficient force to twist a portion of the collagenous tissue by at least 45°, while the pins are inserted into the collagenous tissue; and
wherein movement of the inserted pins to twist the collagenous tissue also rotates the pins and the collagenous tissue relative to the housing.

2. The annuloplasty device of claim 1, wherein the movement of the inserted pins rotates collagenous tissue extending along an axis connecting two of the plurality of pins.

3. The annuloplasty device of claim 2, configured to limit rotation of the tissue extending along the axis to a predetermined maximum rotation relative to the housing; wherein said predetermined maximum rotation is at least one of:
a. of less than 180°;
b. less than 110°;
c. greater than about 70°.

4-5. (canceled)

6. The annuloplasty device of claim 2, wherein the plurality of pins consists of exactly two pins.

7. The annuloplasty device of claim 2, wherein the two of the plurality of pins are positioned with a distance between their axial centers of 1-2 mm.

8. The annuloplasty device of claim 1, comprising a control actuatable from a proximal side of the catheter body to induce the twisting of the portion of collagenous tissue and the movement of the pins relative to a proximal-distal axis of the housing.

9. The annuloplasty device of claim 8, wherein the control comprises an element adjusted by manual manipulation, and the annuloplasty device limits rotation of the pins and the collagenous tissue relative to the housing by limiting movement of the manually manipulated element.

10. The annuloplasty device of claim 8, wherein the control operates by releasing stored energy to perform the rotation, and the annuloplasty device limits rotation of the pins and the collagenous tissue relative to the housing by limiting an amount of the stored energy.

11. The annuloplasty device of claim 1, wherein a distal face of the housing comprises at least one aperture through which at least one of the pins extends; and the annuloplasty device limits rotation of the pins and the collagenous tissue relative to the housing by interference between at least one of the pins and a circumference of the aperture.

12. The annuloplasty device of claim 11, wherein the distal face of the housing comprises a plurality of apertures, through each of which a respective pin extends; and the annuloplasty device limits rotation of the pins and collagenous tissue relative to the housing by interference between at least one of the pins and the circumference of its respective aperture.

13. The annuloplasty device of claim 1, wherein rotation of the pins relative to the housing is limited by an interfering contact with a stop element within the housing; and wherein the interfering contact with stop element comprises interference between an assembly which rotates the pins, and a portion of the housing of the assembly acting as the stop element.

14. (canceled)

15. The annuloplasty device of claim 1, wherein the rotation of the plurality of pins relative to the housing exerts at least 0.01 N·m of torque on the collagenous tissue.

16. The annuloplasty device of claim 1, wherein the plurality of pins exert enough torque on the collagenous tissue to drag a portion of it through a rotation about as large as the rotation of an axis extending between two of the pins, the rotation being relative to a proximal-distal axis of the housing.

17. The annuloplasty device of claim 1, wherein force to actuate the rotation is transmitted along the catheter body by rotation of a control member; and wherein the annuloplasty device limits the rotation by limiting rotation of the control member.

18-20. (canceled)

21. The annuloplasty device of claim 1, wherein the pins are configured for at least one the following:
a. remain at a fixed distance from one another during the rotation;
b. to change their distance from each other during the rotation.

22. (canceled)

23. The annuloplasty device of claim 1, wherein the structurally disruptive energy comprises RF energy transmitted to the tip along a conductive wire;
wherein the pins are electrodes, interconnected with a power connection on a proximal side of the annuloplasty device through the conductive wire; and
wherein the conductive wire is operable to transmit the RF energy to an electrode positioned to contact tissue alongside the pins, when the pins are inserted into the collagenous tissue.

24-25. (canceled)

26. The annuloplasty device of claim 1, wherein the pins are retractable and extendable relative to the housing.

27. The annuloplasty device of claim 1, comprising a steering sheath, through which the catheter is configured to advance to reach target collagenous tissue.

28. The annuloplasty device of claim 27, wherein the steering sheath is configured to one or more of:
a. move coordinately to adopt a curl and bend out of a plane of the curl as the steering sheath is actuated;
b. bend from a straightened configuration to form a segment of the steering sheath into a helical shape.

29. (canceled)

30. The annuloplasty device of claim 28 wherein the helical shape comprises a distal segment of the steering sheath, attached to a more proximal segment of the steering sheath, and the more proximal segment of the steering sheath bends from the straightened configuration to form a curvature, while remaining substantially within a plane of the curvature.

31-50. (canceled)
